SEQUENCE LISTING (135 sequences)

SEQ ID NO: 1 - protein, 5 aa, synthetic construct
GYYWN

SEQ ID NO: 2 - protein, 16 aa, synthetic construct
EINHSGSTSYNPSLKS

SEQ ID NO: 3 - protein, 9 aa, synthetic construct
VLYNWNVDS

SEQ ID NO: 4 - protein, 11 aa, synthetic construct
RASQDIRNDVG

SEQ ID NO: 5 - protein, 7 aa, synthetic construct
AASRLQS

SEQ ID NO: 6 - protein, 9 aa, synthetic construct
LQHNSYPRT

SEQ ID NO: 7 - protein, 5 aa, synthetic construct
GYYWN

SEQ ID NO: 8 - protein, 16 aa, synthetic construct
EINHSGSTNYNPSLKS

SEQ ID NO: 9 - protein, 9 aa, synthetic construct
NLYNWNLDS

SEQ ID NO: 10 - protein, 11 aa, synthetic construct
RASQGIRNDLG

SEQ ID NO: 11 - protein, 7 aa, synthetic construct
VASSLQS

SEQ ID NO: 12 - protein, 9 aa, synthetic construct
LQHNSYPRT

SEQ ID NO: 13 - protein, 7 aa, synthetic construct
SGGYYWS

SEQ ID NO: 14 - protein, 16 aa, synthetic construct
NIYYSGSTYYNPSLKS

SEQ ID NO: 15 - protein, 20 aa, synthetic construct
EKALATVTPSGYENYYTVDV

SEQ ID NO: 16 - protein, 11 aa, synthetic construct
WASQGISSYLA

SEQ ID NO: 17 - protein, 7 aa, synthetic construct
AASTLQS

SEQ ID NO: 18 - protein, 9 aa, synthetic construct
QHLNSYPLT

SEQ ID NO: 19 - protein, 7 aa, synthetic construct
SGGYYWS

SEQ ID NO: 20 - protein, 16 aa, synthetic construct
NIYYSGSTYYNPSLKS

SEQ ID NO: 21 - protein, 20 aa, synthetic construct
EKALASVIPSGYENYYVVDV

SEQ ID NO: 22 - protein, 11 aa, synthetic construct
WASQGIAGYLA

SEQ ID NO: 23 - protein, 7 aa, synthetic construct
AASTLQS

SEQ ID NO: 24 - protein, 9 aa, synthetic construct
QHLNSYPLT

SEQ ID NO: 25 - protein, 5 aa, synthetic construct
DYYMS

SEQ ID NO: 26 - protein, 17 aa, synthetic construct
YISSSGSTIYYTDSVKG

SEQ ID NO: 27 - protein, 7 aa, synthetic construct
DGVGFDY

SEQ ID NO: 28 - protein, 12 aa, synthetic construct
RASQSVSSSYLA

SEQ ID NO: 29 - protein, 7 aa, synthetic construct
AASSRAT

SEQ ID NO: 30 - protein, 10 aa, synthetic construct
QQYGSSPLYT

SEQ ID NO: 31 - protein, 117 aa, synthetic construct
QVQLQQWGAGLLKPSETLSLTCAVYGGSFSGYYWNWIRQPPGKGLEWIGEINHSGSTNYNPSLKSRVTILVDTSKNQFSLKLSSVTAADTAVYYCARNLYNWNLDSWGQGTLVTVSS

SEQ ID NO: 32 - protein, 107 aa, synthetic construct
DIQMTQSPSSLSASVGDRVTITCRASQGIRNDLGWYQQKPGRAPKRLIYVASSLQSGVPSRFSGSGSGTEFTLTISSLQPEDFATYYCLQHNSYPRTFGQGTKVEIK

SEQ ID NO: 33 - protein, 117 aa, synthetic construct
QVQLQQWGAGLLKPSETLSLTCAVYGGSFSGYYWNWIRQPPGKGLEWIGEINHSGSTSYNPSLKSRVTISVDTSKNQFSLKLSSVTAADTAVYYCARVLYNWNVDSWGQGTLVTVSS

SEQ ID NO: 34 - protein, 107 aa, synthetic construct
DIQMTQSPSSLSASVGDRVTITCRASQDIRNDVGWYQQKPGKAPKRLIYAASRLQSGVPSRFSGSGSGTEFTLTISSLQPEDFATYYCLQHNSYPRTFGQGTKVEIK

SEQ ID NO: 35 - protein, 130 aa, synthetic construct
QVQLQESGPGLVKPSQTLSLTCTVSDGSISSGGYYWSWIRQHPGKGLEWIGNIYYSGSTYYNPSLKSRVTISVDTSKNQFSLKLNSVTAADTAVYYCATEKALATVTPSGYENYYTVDVWGQGTTVTVSS

SEQ ID NO: 36 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQHLNSYPLTFGGGTKVEIK

SEQ ID NO: 37 - protein, 130 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCAREKALASVIPSGYENYYVVDVWGQGTTVTVSS

SEQ ID NO: 38 - protein, 107 aa, synthetic construct
DIQLTQSPSFLSASVGGRVTITCWASQGIAGYLAWYQQKPGKAPKLLIYAASTLQSGVPSRFSGSGSGTEFTLTISSLQPEDFATYYCQHLNSYPLTFGGGTKVEIK

SEQ ID NO: 39 - protein, 116 aa, synthetic construct
QVQLVESGGVLVKPGGSLRLSCAASGFTLSDYYMSWIRQAPGMGLEWVSYISSSGSTIYYTDSVKGRFTISRDSAKNSLYLQMNSLRAEDTAVYYCARDGVGFDYWGQGTLVTVSS

SEQ ID NO: 40 - protein, 109 aa, synthetic construct
EIVLTQSPGTLSLFPGERATLSCRASQSVSSSYLAWYQQKPGQSPRLLIYAASSRATGIPDRFSGSGSGTDFTLTISRLEPEDFAVYYCQQYGSSPLYTFGQGTKLEIK

SEQ ID NO: 41 - protein, 331 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 107 aa, synthetic construct [sequence omitted]

SEQ ID NO: 43 - protein, 117 aa, synthetic construct
QVQLQQWGAGLLKPSETLSLTCTVYGGSFSGYYWNWIRQPPGRGLEWIGEINHSGSTSYNPSLKSRITISIDTSKNQFSLKLSSVTAADTAVYYCARVLYNWNVDSWGQGTLVTVSS

SEQ ID NO: 44 - protein, 214 aa, synthetic construct
DIQMTQSPSSLSASVGDRVTITCRASQDIRNDVGWYQQKPGKAPKRLIYAASRLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 117 aa, synthetic construct
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSS

SEQ ID NO: 46 - protein, 117 aa, synthetic construct
QVQLQQWGAGLLKPSETLSLACTVYGGSFSGYYWNWIRQPPGKGLEWIGEIYHSGSTSYNPSLKSRVTISVDTSKNQFSLKLSSVTAADTAVYYCARVLYNWNVDSWGQGTLVTVSS

SEQ ID NO: 47 - protein, 117 aa, synthetic construct
QVQLQQWGAGLLKPSETLSLACTVYGGSFSGYYWNWIRQPPGKGLEWIGEIQHSGSTSYNPSLKSRVTISVDTSKNQFSLKLSSVTAADTAVYYCARVLYNWNVDSWGQGTLVTVSS

SEQ ID NO: 48 - protein, 448 aa, synthetic construct
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 448 aa, synthetic construct
QVQLQQWGAGLLKPSETLSLACTVYGGSFSGYYWNWIRQPPGKGLEWIGEIYHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 448 aa, synthetic construct
QVQLQQWGAGLLKPSETLSLACTVYGGSFSGYYWNWIRQPPGKGLEWIGEIQHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 447 aa, synthetic construct
QVQLQQWGAGLLKPSETLSLACTVYGGSFSGYYWNWIRQPPGKGLEWIGEINHSGSTSYNPSLKSRVTISVDTSKNQFSLKLSSVTAADTAVYYCARVLYNWNVDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 331 aa, synthetic construct; identical to SEQ ID NO: 41

SEQ ID NO: 53 - DNA, 858 bp, Homo sapiens
  source 1..858: mol_type = genomic DNA
gccaccatggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataataa

SEQ ID NO: 54 - DNA, 846 bp, Homo sapiens
  source 1..846: mol_type = genomic DNA
atggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaa

SEQ ID NO: 55 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 56 - protein, 5 aa, synthetic construct
DYGMH

SEQ ID NO: 57 - protein, 17 aa, synthetic construct
YISSGSYTIYSADSVKG

SEQ ID NO: 58 - protein, 13 aa, synthetic construct
RAPNSFYEYYFDY

SEQ ID NO: 59 - protein, 16 aa, synthetic construct
SASSKHTNLYWSRHMY

SEQ ID NO: 60 - protein, 7 aa, synthetic construct
LTSNRAT

SEQ ID NO: 61 - protein, 9 aa, synthetic construct
QQWSSNPFT

SEQ ID NO: 62 - protein, 122 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSS

SEQ ID NO: 63 - protein, 452 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 112 aa, synthetic construct
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIK

SEQ ID NO: 65 - protein, 219 aa, synthetic construct
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 5 aa, synthetic construct
SYAMH

SEQ ID NO: 67 - protein, 17 aa, synthetic construct
VISYAGEVKYYADSVKG

SEQ ID NO: 68 - protein, 12 aa, synthetic construct
DPLPLHYYGMDV

SEQ ID NO: 69 - protein, 13 aa, synthetic construct
SGSSSNIGRRPVN

SEQ ID NO: 70 - protein, 7 aa, synthetic construct
SQNQRPS

SEQ ID NO: 71 - protein, 11 aa, synthetic construct
AVWDDIGRVLQ

SEQ ID NO: 72 - protein, 121 aa, synthetic construct
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSS

SEQ ID NO: 73 - protein, 451 aa, synthetic construct
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 110 aa, synthetic construct
QSVLTQPPSASGTPGQRVTISCSGSSSNIGRRPVNWYQQLPGTAPKLLIYSQNQRPSGVPDRFSGSQSGTSASLAISGLQSEDEADYFCAVWDDIGRVLQLGGGTQLTVL

SEQ ID NO: 75 - protein, 216 aa, synthetic construct
QSVLTQPPSASGTPGQRVTISCSGSSSNIGRRPVNWYQQLPGTAPKLLIYSQNQRPSGVPDRFSGSQSGTSASLAISGLQSEDEADYFCAVWDDIGRVLQLGGGTQLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 76 - DNA, 366 bp, synthetic construct
  source 1..366: mol_type = other DNA
gaggtgcagctggtggaatccggcggaggactggtgcagcctggcggctccctgagactgtcttgcgccgcctccggcttcacattctccgactacggcatgcactgggtccgacaggcccctggaaagggcctggaatgggtggcctacatctcctccggctcctacaccatctactccgccgactccgtgaagggccggttcaccatctcccgggacaacgccaagaactccctgtacctgcagatgaactccctgcgggccgaggacacagccgtgtactactgtgccagacgggcccctaactccttctacgagtactacttcgactactggggccagggcaccaccgtgaccgtgtcctct

SEQ ID NO: 77 - DNA, 1359 bp, synthetic construct
  source 1..1359: mol_type = other DNA
gaggtgcagctggtggaatccggcggaggactggtgcagcctggcggctccctgagactgtcttgcgccgcctccggcttcacattctccgactacggcatgcactgggtccgacaggcccctggaaagggcctggaatgggtggcctacatctcctccggctcctacaccatctactccgccgactccgtgaagggccggttcaccatctcccgggacaacgccaagaactccctgtacctgcagatgaactccctgcgggccgaggacacagccgtgtactactgtgccagacgggcccctaactccttctacgagtactacttcgactactggggccagggcaccaccgtgaccgtgtcctctgctagcaccaaaggtccgagcgtttttccgctggcaccgagcagcaaaagcacctctggtggcaccgcagcactgggttgtctggtgaaagattattttccggaaccggttaccgtttcttggaatagcggtgcactgaccagcggtgttcatacctttccggcagttctgcagagcagcggtctgtatagcctgtctagcgttgttaccgttccgagcagcagcctgggcacccagacctatatttgcaatgtgaatcataaaccgagcaatacaaaagttgataaacgcgttgaaccgaaaagctgtgacaaaactcacacgtgcccaccgtgcccagcacctgagttcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccagcatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 78 - DNA, 336 bp, synthetic construct
  source 1..336: mol_type = other DNA
cagatcgtgctgacccagtcccctgccaccctgtccctgagccctggcgagagagccaccctgagctgctccgcctcctccaagcacaccaacctgtactggtcccggcacatgtactggtatcagcagaagcccggccaggcccctcggctgctgatctacctgacctctaaccgggccaccggcatccctgccagattctccggctctggctccggcaccgacttcaccctgaccatctccagcctggaacccgaggacttcgccgtgtactactgccagcagtggtcctccaaccccttcaccttcggccagggcaccaagctggaaatcaag

SEQ ID NO: 79 - DNA, 660 bp, synthetic construct
  source 1..660: mol_type = other DNA
cagatcgtgctgacccagtcccctgccaccctgtccctgagccctggcgagagagccaccctgagctgctccgcctcctccaagcacaccaacctgtactggtcccggcacatgtactggtatcagcagaagcccggccaggcccctcggctgctgatctacctgacctctaaccgggccaccggcatccctgccagattctccggctctggctccggcaccgacttcaccctgaccatctccagcctggaacccgaggacttcgccgtgtactactgccagcagtggtcctccaaccccttcaccttcggccagggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 80 - DNA, 363 bp, synthetic construct
  source 1..363: mol_type = other DNA
caggtgcagctggtggagtctggaggaggcgtggtccagcctgggaggtccctgagactctcctgtgcagcctctggattcaccttcagtagctatgctatgcactgggtccgccaggctccaggcaaggggctggagtgggtggcagttatatcatatgcgggggaggtgaaatactacgcagactccgtgaagggccgattcaccatctccagagacaattccaagaacacgctgtatctgcaaatgaacagcctgagagctgaggacacggctgtgtattactgtgcgagagacccgctaccgctacattactacggtatggacgtctggggccaagggaccacggtcaccgtctcctca

SEQ ID NO: 81 - DNA, 1356 bp, synthetic construct
  source 1..1356: mol_type = other DNA
caggtgcagctggtggagtctggaggaggcgtggtccagcctgggaggtccctgagactctcctgtgcagcctctggattcaccttcagtagctatgctatgcactgggtccgccaggctccaggcaaggggctggagtgggtggcagttatatcatatgcgggggaggtgaaatactacgcagactccgtgaagggccgattcaccatctccagagacaattccaagaacacgctgtatctgcaaatgaacagcctgagagctgaggacacggctgtgtattactgtgcgagagacccgctaccgctacattactacggtatggacgtctggggccaagggaccacggtcaccgtctcctcagcgtcgaccaaaggtccgagcgtgtgcccgctggcaccgagcagcaaaagcacctctggtggcaccgcagcactgggttgtctggtgaaagattattttccggaaccggttaccgtttcttggaatagcggtgcactgaccagcggtgttcatacctttccggcagtcctgcagagcagcggtctgtatagcctgtctagcgttgttaccgttccgagcagcagcctgggcacccagacctatatttgcaatgtgaatcataaaccgagcaataccaaagttgataaacgcgttgaaccgaaaagcgtggacaaaactcacacgtgcccaccgtgcccagcacctgagttcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccagcatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaatga

SEQ ID NO: 82 - DNA, 330 bp, synthetic construct
  source 1..330: mol_type = other DNA
cagtctgtgctgactcagcctccctcagcgtctgggacccccgggcagagggtcaccatctcttgttctggaagcagctccaatatcggaaggaggcctgtaaactggtaccagcagctcccaggaacggcccccaaactcctcatctatagtcagaatcagcggccctcaggggtccctgaccgattctctggctcccagtctggcacctcagcctccctggccatcagtgggctccagtctgaggatgaggctgattatttctgtgcagtttgggatgacatcggccgcgtcctgcagttaggcggaggcacccagctgaccgtccta

SEQ ID NO: 83 - DNA, 651 bp, synthetic construct
  source 1..651: mol_type = other DNA
cagtctgtgctgactcagcctccctcagcgtctgggacccccgggcagagggtcaccatctcttgttctggaagcagctccaatatcggaaggaggcctgtaaactggtaccagcagctcccaggaacggcccccaaactcctcatctatagtcagaatcagcggccctcaggggtccctgaccgattctctggctcccagtctggcacctcagcctccctggccatcagtgggctccagtctgaggatgaggctgattatttctgtgcagtttgggatgacatcggccgcgtcctgcagttaggcggaggcacccagctgaccgtcctaggtcagcccaaggcggccccctcggtcactctgttcccgccctgctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaagtgtcatga

SEQ ID NO: 84 - protein, 5 aa, synthetic construct
SYAMS

SEQ ID NO: 85 - protein, 17 aa, synthetic construct
AISGSGGSTYYADSVKG

SEQ ID NO: 86 - protein, 13 aa, synthetic construct
GSYGTYYGNYFEY

SEQ ID NO: 87 - protein, 11 aa, synthetic construct
GGDNIGGKSVH

SEQ ID NO: 88 - protein, 7 aa, synthetic construct
YDSDRPS

SEQ ID NO: 89 - protein, 11 aa, synthetic construct
QVLDRRSDHFL

SEQ ID NO: 90 - protein, 11 aa, synthetic construct
QVLDRRSDHWL

SEQ ID NO: 91 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGQGTLVTVSS

SEQ ID NO: 92 - protein, 452 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 108 aa, synthetic construct
QTVLTQPPSVSVAPGKTASISCGGDNIGGKSVHWYQQKPGQAPVLVIYYDSDRPSGIPQRFSGSNSGNTATLTIHRVEAGDEADYYCQVLDRRSDHWLFGGGTKLTVL

SEQ ID NO: 94 - protein, 108 aa, synthetic construct
SYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGGGTKLTVL

SEQ ID NO: 95 - protein, 214 aa, synthetic construct
SYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 96 - protein, 710 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVWDVSHEDPEVKFNWYVDGVEVHNAKTKPREEQYNSTYRWSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARGSYGTYYGNYFEYWGQGTLVTVSSGGGGSGGGGSGGGGSGGGGSSYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGCGTKLTVL

SEQ ID NO: 97 - protein, 112 aa, synthetic construct; identical to SEQ ID NO: 64

SEQ ID NO: 98 - protein, 720 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVWDVSHEDPEVKFNWYVDGVEVHNAKTKPREEQYNSTYRWSVLTVLHQDWLNGKEYKCKVSNKALPASIEKTISKAKGQPREPQVYTLPPSREEMTKNQVSLTCLVKGFYPSDIAVEWESNGGGGSGGGGSEVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARGSYGTYYGNYFEYWGQGTLVTVSSGGGGSGGGGSGGGGSGGGGSSYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGCGTKLTVLGGGGSGGGGSGQPENNYKTTPPVLDSDGSFFLYSKLTVDKSRWQQGNVFSCSVMHEALHNHYTQKSLSLSPGK

SEQ ID NO: 99 - protein, 218 aa, synthetic construct
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGRGTLVTVSS

SEQ ID NO: 101 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 102 - protein, 109 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIK

SEQ ID NO: 103 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 104 - protein, 213 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 105 - protein, 455 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 10 aa, synthetic construct
GFTFSDYGMH

SEQ ID NO: 107 - protein, 17 aa, synthetic construct
YISSGSYTIYSADSVKG

SEQ ID NO: 108 - protein, 12 aa, synthetic construct
RAPNSFYEYYFD

SEQ ID NO: 109 - protein, 10 aa, synthetic construct
GFTFSSYGMH

SEQ ID NO: 110 - protein, 17 aa, synthetic construct
VIWYDGSNKYYADSVKG

SEQ ID NO: 111 - protein, 16 aa, synthetic construct
DPRGATLYYYYYGMDV

SEQ ID NO: 112 - protein, 11 aa, synthetic construct
RASQSINSYLD

SEQ ID NO: 113 - protein, 7 aa, synthetic construct
AASSLQS

SEQ ID NO: 114 - protein, 7 aa, synthetic construct
QQYYSTP

SEQ ID NO: 115 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 116 - protein, 5 aa, synthetic construct
DNDFS

SEQ ID NO: 117 - protein, 17 aa, synthetic construct
AIVAVFRTETYAQKFQD

SEQ ID NO: 118 - protein, 15 aa, synthetic construct
RLMSAISGPGAPLLM

SEQ ID NO: 119 - protein, 14 aa, synthetic construct
TGTSSDVGGYNYVS

SEQ ID NO: 120 - protein, 7 aa, synthetic construct
DVSKRPS

SEQ ID NO: 121 - protein, 11 aa, synthetic construct
SSYTSSDTLEI

SEQ ID NO: 122 - protein, 5 aa, synthetic construct
DYYIH

SEQ ID NO: 123 - protein, 17 aa, synthetic construct
WMNPNSGNTGYAQKFQG

SEQ ID NO: 124 - protein, 7 aa, synthetic construct
GQGYTHS

SEQ ID NO: 125 - protein, 11 aa, synthetic construct
RASEGIYHWLA

SEQ ID NO: 126 - protein, 7 aa, synthetic construct
KASSLAS

SEQ ID NO: 127 - protein, 9 aa, synthetic construct
QQYSNYPPT

SEQ ID NO: 128 - protein, 124 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSS

SEQ ID NO: 129 - protein, 454 aa, synthetic construct
QVQLVQSGAEVKKPGSSVKVSCKASGGTFSDNDFSWVRQAPGQGLEWMGAIVAVFRTETYAQKFQDRVKITADISTRTTYMELSSLRSEDTAVYYCARRLMSAISGPGAPLLMWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 122 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVLGQPKAAPSVTL

SEQ ID NO: 131 - protein, 217 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYTSSDTLEIFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 132 - protein, 116 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSS

SEQ ID NO: 133 - protein, 446 aa, synthetic construct
QVQLVQSGAEVKKPGASVKVSCKASGYTFTDYYIHWVRQATGQGLEWMGWMNPNSGNTGYAQKFQGRVTMTRDTSISTAYMELSSLRSEDTAVYYCARGQGYTHSWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 134 - protein, 107 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIK

SEQ ID NO: 135 - protein, 214 aa, synthetic construct
DIQMTQSPSTLSASVGDRVTITCRASEGIYHWLAWYQQKPGKAPKLLIYKASSLASGVPSRFSGSGSGTEFTLTISSLQPDDFATYYCQQYSNYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0075160 A1
(43) Pub. Date: Mar. 7, 2024

(54) COMBINATION THERAPIES FOR TREATMENT OF CANCER

(71) Applicant: MedImmune, LLC, Gaithersburg, MD (US)

(72) Inventors: Bilal OMAR, Gaithersburg, MD (US); Scott A. HAMMOND, Gaithersburg, MD (US); Judith ANDERTON, Cambridge (GB); Nadia LUHESHI, Cambridge (GB); Daniel J. FREEMAN, Gaithersburg, MD (US); Krista KINNEER, Gaithersburg, MD (US); Jorge ZERON-MEDINA CUAIRAN, Gatihersbrug, MD (US); Ikbel ACHOUR, Gaithersburg, MD (US); Doug PALMER, Gaithersburg, MD (US)

(21) Appl. No.: 18/360,542

(22) Filed: Jul. 27, 2023

Related U.S. Application Data
(60) Provisional application No. 63/369,718, filed on Jul. 28, 2022.

(51) Int. Cl. A61K 47/68; A61K 39/395; A61P 35/00

(52) U.S. Cl. CPC A61K 47/6869; A61K 39/3955; A61K 47/68035; A61K 47/6889; A61P 35/00

(57) ABSTRACT

Provided herein are methods of treating cancer in a human subject, comprising administering to the subject: i) an antibody-drug conjugate (ADC) comprising an antibody, a cleavable linker and a cytotoxic agent, and ii) a bispecific checkpoint inhibitor. Further provided herein are kits comprising i) an antibody-drug conjugate (ADC) comprising an antibody, a cleavable linker and a cytotoxic agent, and ii) a bispecific checkpoint inhibitor.

### CROSS-REFERENCE TO RELATED APPLICATIONS

This application claims the priority benefit of U.S. Provisional Application No. 63/369,718 filed Jul. 28, 2022, which is incorporated herein by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 137,419 Byte file named “ADCIO-101-US-NP.xml” created on Jul. 24, 2023.

### FIELD

The present disclosure provides methods of treating cancer in a human subject, comprising administering to the subject an antibody-drug conjugate (ADC) and a bispecific binding protein. The present disclosure further provides kits comprising an ADC and a bispecific binding protein.

### BACKGROUND

Surgery, radiation therapy and chemotherapy, alone or in combination, are the most traditional and widely used treatment methods for cancer. While these treatment methods are effective in removing or killing cancer cells, they often result in undesirable side effects such as hair loss, anemia, severe nausea and death of healthy cells in patients treated. These limitations present an urgent need for innovative and less harmful treatment methods of cancer. Antibody based cancer therapies rely on the recognition and binding of antibody-drug conjugates to specific proteins on cancer cells. Antibody drug conjugates (ADCs) can take advantage of the specificity of the antibody portion of the conjugate to deliver a highly toxic agent directly to the cells to be killed. Using antibody or ADC cancer therapies in combination with other small molecule based cancer therapies can improve treatment outcomes by attacking malignant cells and tumors in more than one way.

### SUMMARY

The present disclosure provides a method of treating cancer in a human subject in need thereof, comprising administering to the human subject: a) an antibody-drug conjugate (ADC) comprising: i) an antibody or antigen binding fragment thereof which binds to a B7-H4 polypeptide, ii) a cleavable linker, and iii) a cytotoxic agent; and b) a bispecific binding protein having a first binding domain that specifically binds to Programmed cell death protein 1 (PD-1).

In some aspects, the present disclosure is directed to a method of treating cancer in a human subject in need thereof, comprising administering to the human subject: a) an antibody-drug conjugate (ADC) comprising: i) an antibody or antigen binding fragment thereof, ii) a cleavable linker, and iii) a cytotoxic agent; wherein the cytotoxic agent is a compound of formula I:

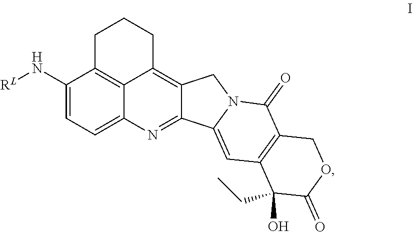

and salts and solvates thereof, wherein R^{L} is the cleavable linker, which is selected from:

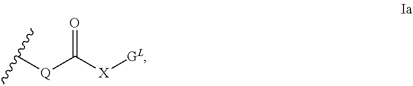

wherein Q is:

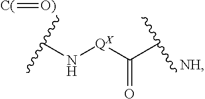

where Q^{X} is such that Q is an amino-acid residue, a dipeptide residue, a tripeptide residue or a tetrapeptide residue;
wherein X is:

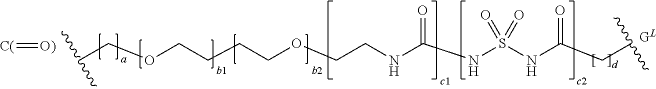

where a=0 to 5, b1=0 to 16, b2=0 to 16, c1=0 or 1, c2=0 or 1, d=0 to 5, at least b1 or b2=0, at least c1 or c2=0; and
wherein G^{L} is a linker for connecting to the antibody or antigen binding fragment; and

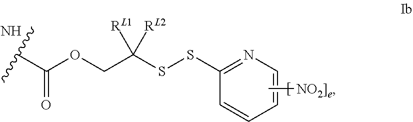

wherein R^{L1} and R^{L2} are independently selected from H and methyl, or together with the carbon atom to which they are bound form a cyclopropylene or cyclobutylene group, and e is 0 or 1; and b) a bispecific binding protein having a first binding domain that specifically binds to PD-1.

In some aspects of the methods, the antibody or antigen binding fragment thereof of the ADC comprises: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof, a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof, a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof, a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof, or a HCDR1, a HCDR2, a HCDR3, a LCDR1, a LCDR2, and a LCDR3 in a first binding arm comprising the amino acid sequence of SEQ ID NO: 116, SEQ ID NO: 117, SEQ ID NO: 118, SEQ ID NO: 119, SEQ ID NO: 120, and SEQ ID NO: 121; and a HCDR1, a HCDR2, a HCDR3, a LCDR1, a LCDR2, and a LCDR3 in a second binding arm comprising the amino acid sequence of SEQ ID NO: 122, SEQ ID NO: 123, SEQ ID NO: 124, SEQ ID NO: 125, SEQ ID NO: 126, and SEQ ID NO: 127.

In some aspects of the methods, the antibody or antigen binding fragment thereof of the ADC comprises: a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof. In some aspects of the methods, the antibody or antigen binding fragment thereof of the ADC is a bispecific antibody or antigen binding fragment thereof that comprises a HCDR1, a HCDR2, a HCDR3, a LCDR1, a LCDR2, and a LCDR3 in a first binding arm comprising the amino acid sequence of SEQ ID NO: 116, SEQ ID NO: 117, SEQ ID NO: 118, SEQ ID NO: 119, SEQ ID NO: 120, and SEQ ID NO: 121; and a HCDR1, a HCDR2, a HCDR3, a LCDR1, a LCDR2, and a LCDR3 in a second binding arm comprising the amino acid sequence of SEQ ID NO: 122, SEQ ID NO: 123, SEQ ID NO: 124, SEQ ID NO: 125, SEQ ID NO: 126, and SEQ ID NO: 127. In some aspects of the methods, the antibody or antigen binding fragment thereof of the ADC comprises: a variable heavy (VH) chain and a variable light (VL) chain comprising the amino acid sequence of SEQ ID NO: 45 and SEQ ID NO: 34, respectively, or a functional variant thereof; a variable heavy (VH) chain and a variable light (VL) chain comprising the amino acid sequence of SEQ ID NO: 33 and SEQ ID NO: 34, respectively, or a functional variant thereof; a variable heavy (VH) chain and a variable light (VL) chain comprising the amino acid sequence of SEQ ID NO: 43 and SEQ ID NO: 34, respectively, or a functional variant thereof; a variable heavy (VH) chain and a variable light (VL) chain comprising the amino acid sequence of SEQ ID NO: 46 and SEQ ID NO: 34, respectively, or a functional variant thereof, a variable heavy (VH) chain and a variable light (VL) chain comprising the amino acid sequence of SEQ ID NO: 47 and SEQ ID NO: 34, respectively, or a functional variant thereof, a VH chain and a VL chain comprising the amino acid sequence of SEQ ID NO: 31, and SEQ ID NO: 32, respectively, or a functional variant thereof, a VH chain and a VL chain comprising the amino acid sequence of SEQ ID NO: 35 and SEQ ID NO: 36, respectively, or a functional variant thereof, a VH chain and a VL chain comprising the amino acid sequence of SEQ ID NO: 37 and SEQ ID NO: 38, respectively, or a functional variant thereof, or a VH chain and a VL chain comprising the amino acid sequence of SEQ ID NO: 39 and SEQ ID NO: 40, respectively, or a functional variant thereof; or a VH chain and a VL chain in a first binding arm comprising the amino acid sequence of SEQ ID NO: 128 and SEQ ID NO: 130, respectively, or a functional variant thereof, and a VH chain and a VL chain in a second binding arm comprising the amino acid sequence of SEQ ID NO: 132 and SEQ ID NO: 134, respectively, or a functional variant thereof.

In some aspects of the methods, the antibody or antigen binding fragment thereof of the ADC comprises: a VH chain and a VL chain comprising the amino acid sequence of SEQ ID NO: 45 and SEQ ID NO: 34, respectively, or a functional variant thereof. In some aspects of the methods, the antibody or antigen binding fragment thereof of the ADC binds an OVCAR4 cell line. In some aspects of the methods, the antibody or antigen binding fragment thereof of the ADC comprises a heavy chain constant region comprising the amino acid sequence of SEQ ID NO: 41.

In some aspects of the methods, the antibody or antigen binding fragment thereof of the ADC is a bispecific antibody or antigen binding fragment thereof and comprises a VH chain and a VL chain in a first binding arm comprising the amino acid sequence of SEQ ID NO: 128 and SEQ ID NO: 130, respectively, or a functional variant thereof, and a VH chain and a VL chain in a second binding arm comprising the amino acid sequence of SEQ ID NO: 132 and SEQ ID NO: 134, respectively, or a functional variant thereof.

In some aspects of the methods, the antibody or antigen binding fragment thereof of the ADC comprises a heavy chain and a light chain in a first binding arm comprising the amino acid sequence of SEQ ID NO: 129 and SEQ ID NO: 131, respectively, or a functional variant thereof, and a heavy chain and a light chain in a second binding arm comprising the amino acid sequence of SEQ ID NO: 133 and SEQ ID NO: 135, respectively, or a functional variant thereof.

In some aspects, the antibody or antigen binding fragment thereof of the ADC comprises a heavy chain constant region comprising the amino acid sequence of SEQ ID NO: 52. In some aspects, the antibody or antigen binding fragment thereof of the ADC comprises a light chain constant region comprising the amino acid sequence of SEQ ID NO: 42. In some aspects, the antibody or antigen binding fragment thereof of the ADC comprises a heavy chain comprising the amino acid sequence of SEQ ID NO: 51; and a light chain comprising the amino acid sequence of SEQ ID NO: 44. In some aspects, the antibody or antigen binding fragment thereof of the ADC comprises a heavy chain comprising the amino acid sequence of SEQ ID NO: 48; and a light chain comprising the amino acid sequence of SEQ ID NO: 44.

In some aspects of the methods, the antibody or antigen binding fragment thereof of the ADC is a monoclonal antibody. In some aspects, the antibody or antigen binding fragment thereof of the ADC is a humanized monoclonal antibody. In some aspects of the methods, the cleavable linker of the ADC is an mp-PEG8-val-ala linker. In some aspects of the methods, the cytotoxic agent of the ADC is topoisomerase inhibitor (TOP1i), tubulysin derivative, a pyrrolobenzodiazepine, or a combination thereof. In some aspects, the cytotoxic agent of the ADC is a topoisomerase inhibitor.

In some aspects, the cytotoxic agent is a topoisomerase inhibitor and wherein the ii) cleavable linker and iii) cytotoxic agent of the ADC comprise a compound of formula I:

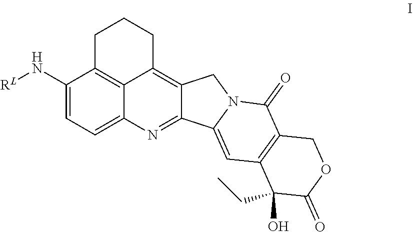

and salts an solvates thereof, wherein is the cleavable inker, which is selected from:

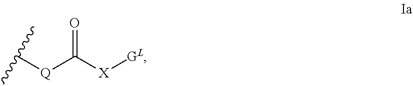

wherein Q is:

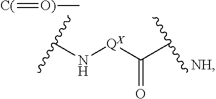

where Q^{X} is such that Q is an amino-acid residue, a dipeptide residue, a tripeptide residue or a tetrapeptide residue;
wherein X is:

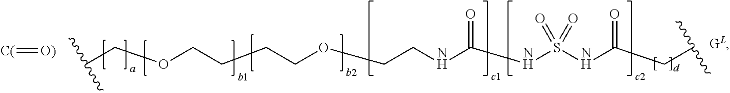

where a=0 to 5, b1=0 to 16, b2=0 to 16, c1=0 or 1, c2=0 or 1, d=0 to 5, at least b1 or b2=0, at least c1 or c2=0; and
   wherein G^{L} is a linker for connecting to the antibody or antigen binding fragment; and

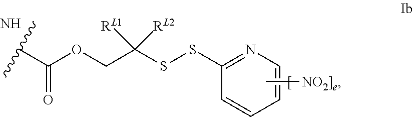

wherein R^{L1} and R^{L2} are independently selected from H and methyl, or together with the carbon atom to which they are bound form a cyclopropylene or cyclobutylene group, and e is 0 or 1.

In some aspects, R^{L} is of formula Ia.

In some aspects, Q is:
(a) an amino acid residue selected from: Phe, Lys, Val, Ala, Cit, Leu, Ile, Arg, and Trp; or
(b) a dipeptide residue selected from: ^{NH}-Phe-Lys-^{C═O}, ^{NH}-Val-Ala-^{C═O}, ^{NH}-Val-Lys-^{C═O}, ^{NH} Ala-Lys-^{C═O}, NH-Val-Cit-^{C═O}, ^{NH}-Phe-Cit-^{C═O}, ^{NH}-Leu-Cit-^{C═O}, ^{NH}-Ile-Cit-^{C═O}, ^{NH}-Phe-Arg-^{C═O}, ^{NH}-Trp-Cit-^{C═O}, and ^{NH}-Gly-Val-^{C═O}; or
(c) a tripeptide residue selected from: ^{NH}-Glu-Val-Ala-^{C═O}, ^{NH}-Glu-Val-Cit-^{C═O}, ^{NH}-αGlu-Val-Ala-^{C═O}, and ^{NH}-αGlu-Val-Cit-^{C═O}; or
(d) a tetrapeptide residue selected from: ^{NH}-Gly-Gly-Phe-Gly ^{C═O}; and ^{NH}-Gly-Phe-Gly-Gly ^{C═O}.

In some aspects, a is: (a) 0 to 3; or (b) 0 or 1; or (c) 0.

In some aspects, b1 is: (a) 0 to 8; or (b) 0; or (c) 2; or (d) 3; or (e) 4; or (f) 5; or (g) 8.

In some aspects, b2 is: (a) 0 to 8; or (b) 0; or (c) 2; or (d) 3; or (e) 4; or (f) 5; or (g) 8.

In some aspects, (a) c1 is 0 or 1; (b) c2 is 0 or 1; and (c) at least one of c1 and c2 is 0.

In some aspects, d is: (a) 0 to 3; or (b) 1 or 2; or (c) 2; or (d) 5.

In some aspects:
(a) a is 0, b1 is 0, c1 is 1, c2 is 0 and d is 2, and b2 is 0, 2, 3, 4, 5 or 8; or
(b) a is 1, b2 is 0, c1 is 0, c2 is 0 and d is 0, and b1 is 0, 2, 3, 4, 5 or 8; or
(c) a is 0, b1 is 0, c1 is 0, c2 is 0 and d is 1, and b2 is 0, 2, 3, 4, 5 or 8; or
(d) b1 is 0, b2 is 0, c1 is 0, c2 is 0, one of a and d is 0, and the other of a and d is 1 or 5; or
(e) a is 1, b2 is 0, c1 is 0, c2 is 1, d is 2, and b1 is 0, 2, 3, 4, 5 or 8.

In some aspects, G^{L} is selected from:

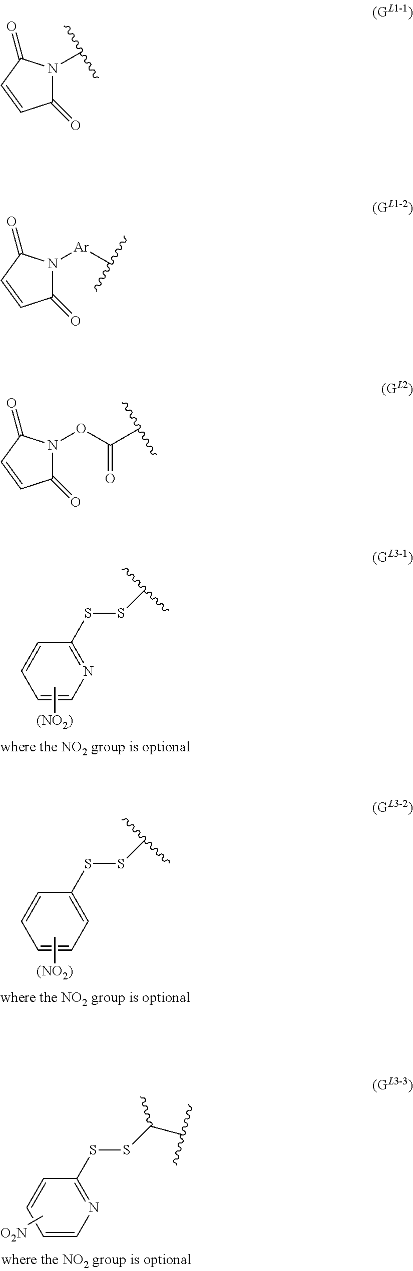

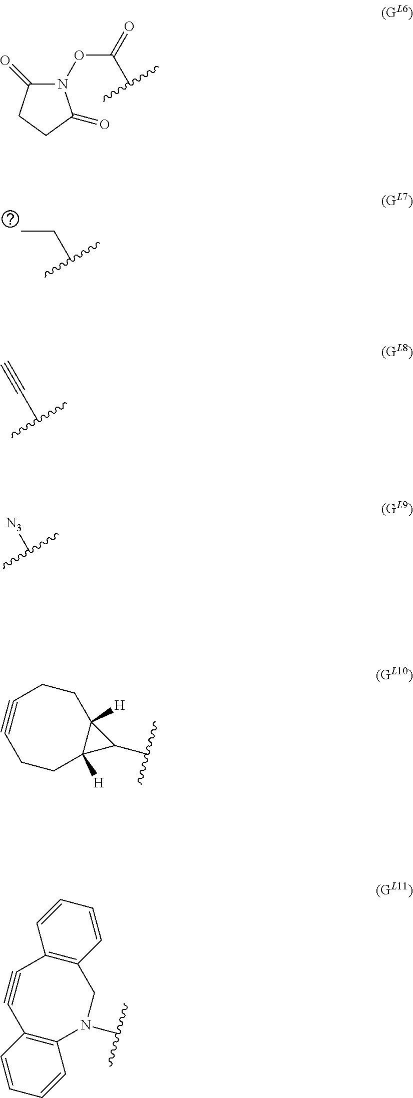

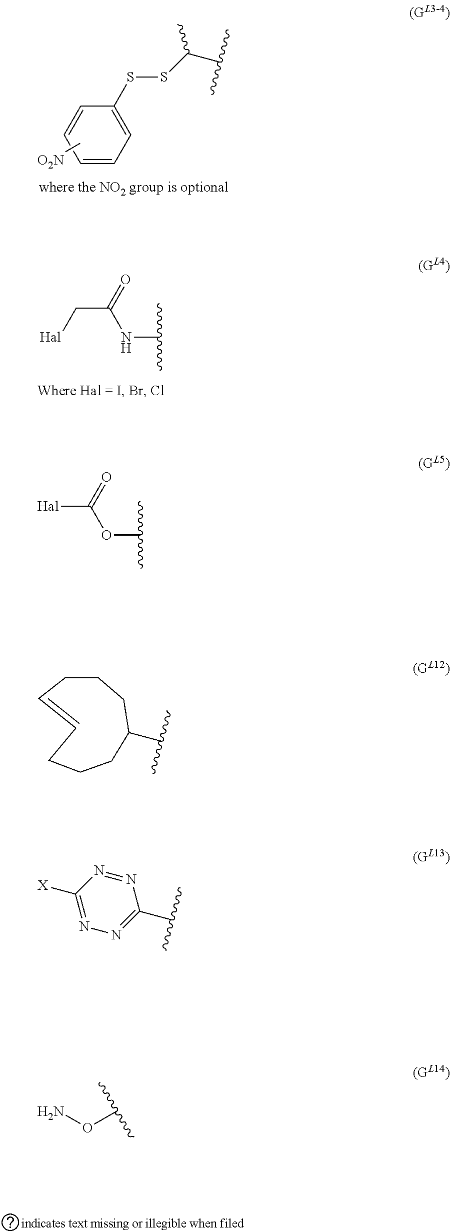

where Ar represents a C₅₋₆ arylene group, and X represents C₁₋₄ alkyl.

In some aspects, G^{L} is selected from G^{L1-1} and G^{L1-2}.

In some aspects, R^{L} is of formula Ib, and:
(a) both R^{L1} and R^{L2} are H; or
(b) R^{L1} is H and R^{L2} is methyl; or
(c) both R^{L1} and R^{L2} are methyl; or
(d) wherein R^{L1} and R^{L2} together with the carbon atom to which they are bound form a cyclopropylene group; or
(e) wherein R^{L1} and R^{L2} together with the carbon atom to which they are bound form a cyclobutylene group.

In some aspects, the ADC is of formula IV:

L-(D^{L})ₚ (IV)
or a pharmaceutically acceptable salt or solvate thereof,
wherein L is the i) antibody or antigen binding fragment thereof, D^{L} is a drug linker unit comprising the ii) cleavable linker and the iii) cytotoxic agent, and D^{L} is of formula III:

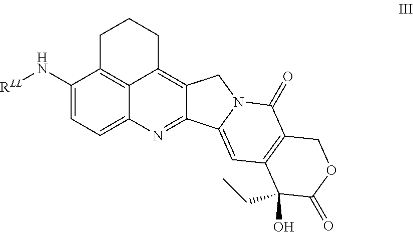

wherein R^{LL} is the cleavable linker selected from: (ia′):

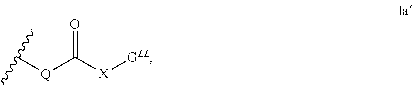

where Q and X are as defined above and G^{LL} is a linker connected to the antibody or antigen binding fragment; and
(ib′):

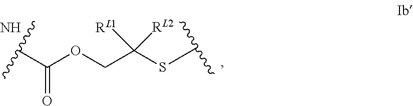

where R^{L1} and R^{L2} are as defined above; and
p is an integer of from 1 to 20.

In some aspects, G^{LL} is selected from:

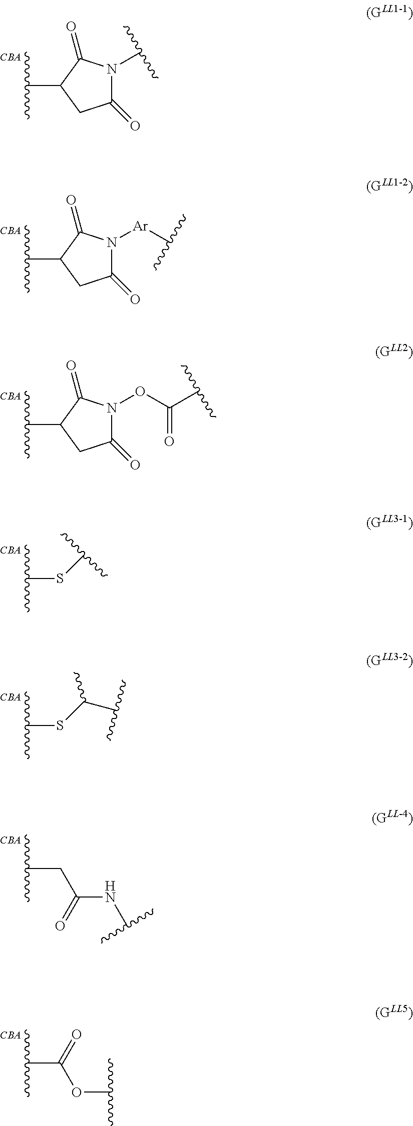

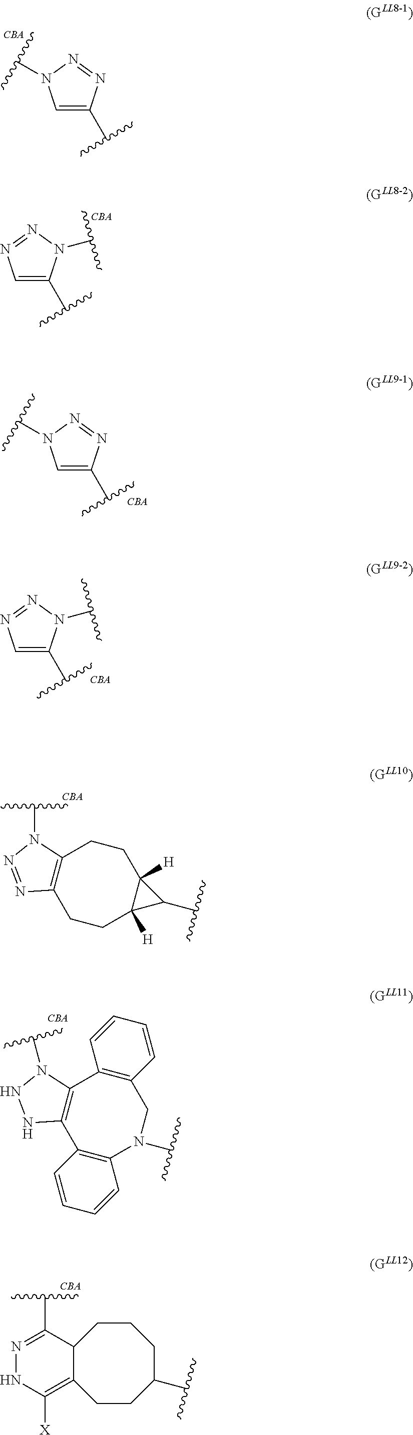

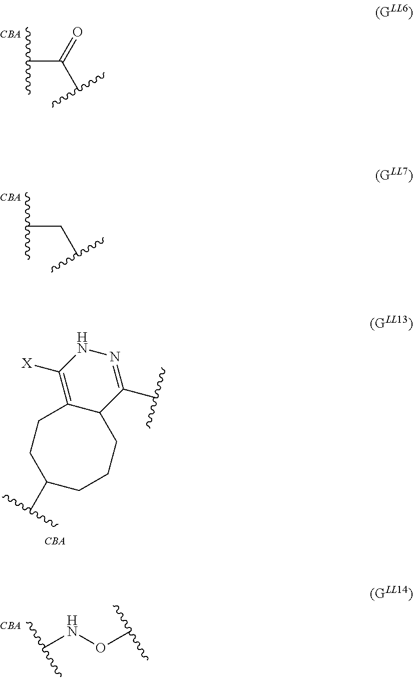

where Ar represents a C₅₋₆ arylene group and X represents C₁₋₄ alkyl.

In some aspects, G^{LL} is selected from G^{LL1-1} and G^{LL1-2}.

In some aspects of the methods, the drug loading (p) of the cytotoxic agent to the antibody or antibody binding fragment is an integer from 1 to about 10.

In some aspects of the methods, the topoisomerase inhibitor is a compound of formula A:

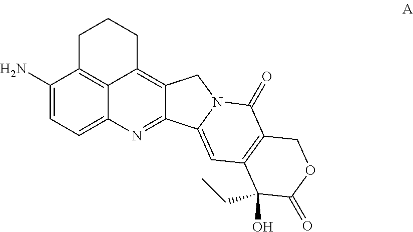 [SMILES: CC[C@@]1(O)C(=O)OCc2c1cc1n(c2=O)Cc2c-1nc1ccc(N)c3c1c2CCC3]

as a single enantiomer or in an enantiomerically enriched form.

In some aspects of the methods, the topoisomerase inhibitor is a compound with the formula VI:

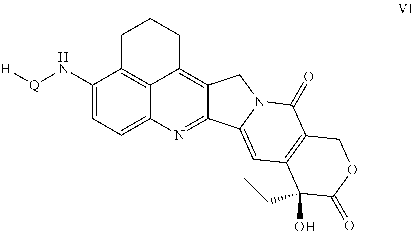

where Q is as defined above.

In some aspects of the methods, the ii) linker and iii) cytotoxic agent together comprise the following compound:

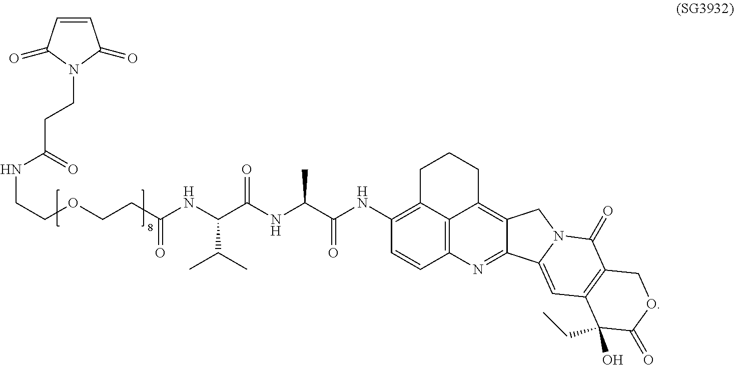

In some aspects of the methods, the bispecific binding protein comprises: a) the first binding domain that specifically binds to PD-1; and b) a second binding domain that specifically binds to T cell immunoreceptor with Ig and ITIM domains (TIGIT), T-cell immunoglobulin and mucin domain containing protein-3 (TIM-3) or cytotoxic T-lymphocyte-associated antigent-4 (CTLA 4). In some aspects of the methods, the first binding domain comprises a heavy chain variable domain comprising a HCDR1 having the amino acid sequence of SEQ ID NO: 56, a HCDR2 having the amino acid sequence of SEQ ID NO: 57, and a HCDR3 having the amino acid sequence of SEQ ID NO: 58, and a light chain variable domain comprising a LCDR1 having the amino acid sequence of SEQ ID NO: 59, a LCDR2 having the amino acid sequence of SEQ ID NO: 60 and a LCDR3 having the amino acid sequence of SEQ ID NO: 61.

In some aspects, the first binding domain that specifically binds to PD-1 comprises a heavy chain variable domain having the amino acid sequence of SEQ ID NO: 62 and a light chain variable domain having the amino acid sequence of SEQ ID NO: 64. In some aspects, the first binding domain that specifically binds to PD-1 comprises a heavy chain variable domain having an amino acid sequence that is at least 90% identical to the amino acid sequence of SEQ ID NO: 7 and a light chain variable domain having an amino acid sequence that is at least 90% identical to the amino acid sequence of SEQ ID NO: 64. In some aspects, the first binding domain that specifically binds to PD-1 comprises a heavy chain having the amino acid sequence of SEQ ID NO: 63 and a light chain having the amino acid sequence of SEQ ID NO: 65. In some aspects, first binding domain that specifically binds to PD-1 comprises a heavy chain having an amino acid sequence that is at least 90% identical to the amino acid sequence of SEQ ID NO: 63 and a light chain having an amino acid sequence that is at least 90% identical to the amino acid sequence of SEQ ID NO: 65. In some aspects of the methods, the second binding domain that specifically binds to TIGIT comprises a heavy chain variable domain comprising a HCDR1 having the amino acid sequence of SEQ ID NO: 66, a HCDR2 having the amino acid sequence of SEQ ID NO: 67, and a HCDR3 having the amino acid sequence of SEQ ID NO: 68, and a light chain variable domain comprising a LCDR1 having the amino acid sequence of SEQ ID NO: 69, a LCDR2 having the amino acid sequence of SEQ ID NO: 70, and a LCDR3 having the amino acid sequence of SEQ ID NO: 71. In some aspects, the second binding domain that specifically binds TIGIT comprises a heavy chain variable domain having the amino acid sequence of SEQ ID NO: 72 and a light chain variable domain having the amino acid sequence of SEQ ID NO: 74. In some aspects, the second binding domain that specifically binds to TIGIT comprises a heavy chain variable domain having an amino acid sequence that is at least 90% identical to the amino acid sequence of SEQ ID NO: 72 and a light chain variable domain having an amino acid sequence that is at least 90% identical to the amino acid sequence of SEQ ID NO: 74. In some aspects, the second binding domain that specifically binds to TIGIT comprises a heavy chain having the amino sequence of SEQ ID NO: 73 and a light chain having the amino acid sequence of SEQ ID NO: 75.

In some aspects, the second binding domain that specifically binds to TIGIT comprises a heavy chain having an amino acid sequence that is at least 90% identical to the amino acid sequence of SEQ ID NO: 73 and a light chain having an amino acid sequence that is at least 90% identical to the amino acid sequence of SEQ ID NO: 75.

In some aspects of the methods, the second binding domain specifically binds to the C′C″ and DE loops of the immunoglobulin variable (IgV) domain of TIM-3 or binds to the PS binding cleft (FG and CC′ loops) of the IgV domain of TIM-3. In some aspects, the second binding domain comprises CDRs: HCDR1, HCDR2, HCDR3, LCDR1, LCDR2, and LCDR3 comprising the amino acid sequences of SEQ ID NOs: 84, 85, 86, 87, 88, and 89, respectively, or SEQ ID NOs: 84, 85, 86, 87, 88, and 90, respectively.

In some aspects, the second binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 102).

In some aspects, the first binding domain comprises a set of CDRs: HCDR1, HCDR2, HCDR3, LCDR1, LCDR2, and LCDR3 comprising the amino acid sequences of SEQ ID NOs: 56, 57, 58, 59, 60, and 61, respectively. In some aspects, the first binding domain comprises a first heavy chain VH comprising the amino acid sequence of SEQ ID NO: 62, and a first light chain VL comprising the amino acid sequence of SEQ ID NO: 64, and the second binding domain comprises a second heavy chain variable domain (VH) comprising the amino acid sequence of SEQ ID NO: 91, and a second light chain variable domain (VL) comprising the amino acid sequence of SEQ ID NO: 94. In some aspects, the first binding domain comprises a first heavy chain comprising the amino acid sequence of SEQ ID NO: 63, and a first light chain comprising the amino acid sequence of SEQ ID NO: 65, and the second binding domain comprises a second heavy chain comprising the amino acid sequence of SEQ ID NO: 92, and a second light chain comprising the amino acid sequence of SEQ ID NO: 95. In some aspects of the methods, the second binding domain specifically binds to CTLA-4. In some aspects, the bispecific binding protein binds to human PD-1 and CTLA-4. In some aspects, the bispecific binding protein comprises the anti-PD-1 and anti-CTLA-4 heavy chain variable region (VH) CDR1, VH CDR2, VH CDR3, light chain variable region (VL) CDR1, VL CDR2, and VL CDR3 of sequences of MEDI5752. In some aspects, the second binding domain comprises a VH CDR1 comprising the amino acid sequence of SEQ ID NO: 109, a VH CDR2 comprising the amino acid sequence of SEQ ID NO: 110, a VH CDR3 comprising the amino acid sequence of SEQ ID NO: 111, a VL CDR1 comprising the amino acid sequence of SEQ ID NO: 112, a VL CDR2 comprising the amino acid sequence of SEQ ID NO: 113, and a VL CDR3 comprising the amino acid sequence of SEQ ID NO: 114. In some aspects, the bispecific checkpoint inhibitor comprises a heavy chain comprising the amino acid sequence set forth in SEQ ID NO: 105 and a light chain comprising the amino acid sequence set forth in SEQ ID NO: 104.

In some aspects, the bispecific binding protein comprises: (a) a VH CDR1 comprising the amino acid sequence of SEQ ID NO: 106, a VH CDR2 comprising the amino acid sequence of SEQ ID NO: 107, a VH CDR3 comprising the amino acid sequence of SEQ ID NO: 108, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; and (b) a VH CDR1 comprising the amino acid sequence of SEQ ID NO: 109, a VH CDR2 comprising the amino acid sequence of SEQ ID NO: 110, a VH CDR3 comprising the amino acid sequence of SEQ ID NO: 111, a VL CDR1 comprising the amino acid sequence of SEQ ID NO: 112, a VL CDR2 comprising the amino acid sequence of SEQ ID NO: 113, and a VL CDR3 comprising the amino acid sequence of SEQ ID NO: 114.

In some aspects, the bispecific binding protein comprises: (a) a heavy chain comprising the amino acid sequence set forth in SEQ ID NO: 63 and a light chain comprising the amino acid sequence set forth in SEQ ID NO: 65; and (b) a heavy chain comprising the amino acid sequence set forth in SEQ ID NO: 105 and a light chain comprising the amino acid sequence set forth in SEQ ID NO: 104.

In some aspects of the methods, the bispecific binding protein comprises an aglycosylated Fc region. In some aspects, the bispecific binding protein comprises a deglycosylated Fc region. In some aspects, the bispecific binding protein comprises an Fc region which has reduced fucosylation or is afucosylated. In some aspects, the bispecific binding protein is a humanized bispecific antibody or antigen-binding fragment thereof. In some aspects, the bispecific binding protein comprises an IgG heavy chain constant region. In some aspects, the constant region includes mutations at L234F, L235E and P331S. In some aspects, the IgG heavy chain constant region is an IgG1 heavy chain constant region. In some aspects, the bispecific binding protein is an antibody. In some aspects, the bispecific binding protein is a full-length antibody. In some aspects, the antibody is an IgG antibody. In some aspects, the antibody is an IgG1 antibody. In some aspects, the antibody is human or humanized. In some aspects, the bispecific binding protein comprises a kappa light chain constant region. In some aspects, the bispecific binding protein comprises a lambda light chain constant region.

In some aspects of the methods, the cancer comprises a cancer cell which expresses B7-H4 polypeptide. In some aspects, the cancer is selected from ovarian cancer, breast cancer, uterine cancer, testicular cancer, bladder cancer, head and neck cancer, melanoma, renal cell carcinoma, pancreatic cancer, prostate cancer, cervical cancer, hematological cancer, endometrial cancer, cholangiocarcinoma, NSCLC (squamous and/or adenocarcinoma), gastrointestinal cancer such as gastric cancer and colorectal cancer, and lung cancer. In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANC1, PALB2, PPP2R2A, RAD51B, RAD51C, RAD51D, and RAD54L. In some aspects, the mutated HRD gene is selected from BRCA1, BRCA2, and ATM.

The present disclosure also provides a pharmaceutical composition comprising: 1) an antibody-drug conjugate and 2) a bispecific checkpoint inhibitor, wherein the antibody-drug conjugate comprises an antibody or antigen binding fragment thereof, and a drug-linker represented by the following formula:

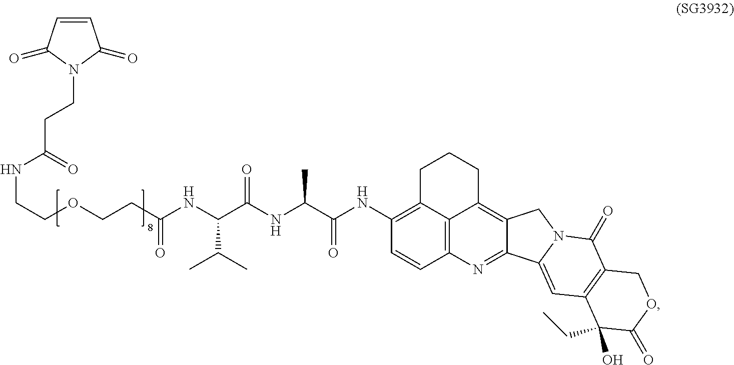

and wherein the drug-linker is conjugated to the antibody or antigen binding fragment thereof. In some aspects, the antibody or antigen binding fragment thereof is an anti-B7H4 antibody. In some aspects, the antibody or antigen binding fragment thereof binds to B7-H4 and comprises: a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6. In some aspects, the antibody or antigen binding fragment thereof comprises a VH chain and a VL chain comprising the amino acid sequence of SEQ ID NO: 45 and SEQ ID NO: 34, respectively, or a functional variant thereof. In some aspects, the antibody or antigen binding fragment thereof comprises a heavy chain comprising the amino acid sequence of SEQ ID NO: 51; and a light chain comprising the amino acid sequence of SEQ ID NO: 44.

In some aspects, the antibody or antigen binding fragment thereof comprises a heavy chain comprising the amino acid sequence of SEQ ID NO: 48; and a light chain comprising the amino acid sequence of SEQ ID NO: 44. In some aspects, the bispecific checkpoint inhibitor comprises: a) a first binding domain that specifically binds to PD-1 or PD-L1; and b) a second binding domain that specifically binds to T cell immunoreceptor with Ig and ITIM domains (TIGIT), T-cell immunoglobulin and mucin domain containing protein-3 (TIM-3) or cytotoxic T-lymphocyte-associated antigent-4 (CTLA 4). In some aspects, the first binding domain specifically binds to PD-1 and comprises a heavy chain variable domain comprising a HCDR1 having the amino acid sequence of SEQ ID NO: 56, a HCDR2 having the amino acid sequence of SEQ ID NO: 57, and a HCDR3 having the amino acid sequence of SEQ ID NO: 58, and a light chain variable domain comprising a LCDR1 having the amino acid sequence of SEQ ID NO: 59, a LCDR2 having the amino acid sequence of SEQ ID NO: 60 and a LCDR3 having the amino acid sequence of SEQ ID NO: 61.

In some aspects, the first binding domain specifically binds to PD-1 and comprises a heavy chain variable domain having the amino acid sequence of SEQ ID NO: 62 and a light chain variable domain having the amino acid sequence of SEQ ID NO: 64. In some aspects, the first binding domain specifically binds to PD-1 and comprises a heavy chain variable domain having an amino acid sequence that is at least 90% identical to the amino acid sequence of SEQ ID NO: 62 and a light chain variable domain having an amino acid sequence that is at least 90% identical to the amino acid sequence of SEQ ID NO: 64. In some aspects, the first binding domain specifically binds to PD-1 and comprises a heavy chain having the amino acid sequence of SEQ ID NO: 63 and a light chain having the amino acid sequence of SEQ ID NO: 65. In some aspects, the first binding domain specifically binds to PD-1 and comprises a heavy chain having an amino acid sequence that is at least 90% identical to the amino acid sequence of SEQ ID NO: 63 and a light chain having an amino acid sequence that is at least 90% identical to the amino acid sequence of SEQ ID NO: 65.

In some aspects, the second binding domain specifically binds to TIGIT and comprises a heavy chain variable domain comprising a HCDR1 having the amino acid sequence of SEQ ID NO: 66, a HCDR2 having the amino acid sequence of SEQ ID NO: 67, and a HCDR3 having the amino acid sequence of SEQ ID NO: 68, and a light chain variable domain comprising a LCDR1 having the amino acid sequence of SEQ ID NO: 69, a LCDR2 having the amino acid sequence of SEQ ID NO: 70, and a LCDR3 having the amino acid sequence of SEQ ID NO: 71. In some aspects, the second binding domain specifically binds TIGIT and comprises a heavy chain variable domain having the amino acid sequence of SEQ ID NO: 72 and a light chain variable domain having the amino acid sequence of SEQ ID NO: 74. In some aspects, the second binding domain specifically binds to TIGIT and comprises a heavy chain variable domain having an amino acid sequence that is at least 90% identical to the amino acid sequence of SEQ ID NO: 72 and a light chain variable domain having an amino acid sequence that is at least 90% identical to the amino acid sequence of SEQ ID NO: 74.

In some aspects, the second binding domain specifically binds to TIGIT and comprises a heavy chain having the amino sequence of SEQ ID NO: 73 and a light chain having the amino acid sequence of SEQ ID NO: 75. In some aspects, the second binding domain specifically binds to TIGIT and comprises a heavy chain having an amino acid sequence that is at least 90% identical to the amino acid sequence of SEQ ID NO: 73 and a light chain having an amino acid sequence that is at least 90% identical to the amino acid sequence of SEQ ID NO: 75. In some aspects, the second binding domain specifically binds to TIM-3. In some aspects, the second binding domain specifically binds to TIM-3, and where in the second binding domain specifically binds to the C′C″ and DE loops of the immunoglobulin variable (IgV) domain of TIM-3 or binds to the PS binding cleft (FG and CC′ loops) of the IgV domain of TIM-3.

In some aspects, the second binding domain comprises Complementarity-Determining Regions (CDRs): HCDR1, HCDR2, HCDR3, LCDR1, LCDR2, and LCDR3 comprising the amino acid sequences of SEQ ID NOs: 84, 85, 86, 87, 88, and 89, respectively, or SEQ ID NOs: 84, 85, 86, 87, 88, and 90, respectively. In some aspects, the second binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 102). In some aspects, the second binding domain comprises a heavy chain variable domain (VH) comprising the amino acid sequence of SEQ ID NO: 91, and a light chain variable domain (VL) comprising the amino acid sequence of SEQ ID NO: 94.

In some aspects, the second binding domain comprises a heavy chain comprising the amino acid sequence of SEQ ID NO: 92, and a light chain comprising the amino acid sequence of SEQ ID NO: 95. In some aspects, the second binding domain specifically binds to CTLA-4. In some aspects, the second binding domain comprises a VH CDR1 comprising the amino acid sequence of SEQ ID NO: 109, a VH CDR2 comprising the amino acid sequence of SEQ ID NO: 110, a VH CDR3 comprising the amino acid sequence of SEQ ID NO: 111, a VL CDR1 comprising the amino acid sequence of SEQ ID NO: 112, a VL CDR2 comprising the amino acid sequence of SEQ ID NO: 113, and a VL CDR3 comprising the amino acid sequence of SEQ ID NO: 114. In some aspects, the bispecific checkpoint inhibitor comprises a heavy chain comprising the amino acid sequence set forth in SEQ ID NO: 105 and a light chain comprising the amino acid sequence set forth in SEQ ID NO: 104.

The present disclosure is also directed to a kit comprising any of the above pharmaceutical compositions. The present disclosure is also directed to use of an antibody-drug conjugate in the manufacture of a medicament for use in combination with a bispecific checkpoint inhibitor, wherein the antibody-drug conjugate and the bispecific checkpoint inhibitor are as defined herein for treating cancer. The present disclosure is also directed to use of a bispecific checkpoint inhibitor in the manufacture of a medicament for use in combination with an antibody-drug conjugate, wherein the bispecific checkpoint inhibitor and the antibody-drug conjugate are as defined herein for treating cancer. In some aspects, the pharmaceutical composition is for use in treating cancer. In some aspects, the cancer is selected from ovarian cancer, breast cancer, uterine cancer, testicular cancer, bladder cancer, head and neck cancer, melanoma, renal cell carcinoma, pancreatic cancer, prostate cancer, cervical cancer, hematological cancer, endometrial cancer, cholangiocarcinoma, NSCLC (squamous and/or adenocarcinoma), gastrointestinal cancer such as gastric cancer and colorectal cancer, and lung cancer.

### BRIEF DESCRIPTION OF THE DRAWINGS

The following drawings form part of the present specification and are included to further demonstrate exemplary embodiments of certain aspects of the present disclosure.

FIGS. 1A-1C show plots of geometric mean fluorescence intensity (gMFI) for expression of different antigens on tumor cells as described in Example 1.

FIGS. 2A-2C show plots of median fluorescence intensity (MFI) for expression of different antigens on dendritic cells as described in Example 1.

FIGS. 3A-3C show plots of percent cytolysis of tumor cells when exposed to tumor antigen specific T-cells and either PD-1-TIGIT bispecific AZD2936 (FIG. 3A),), PD-1-TIM3 bispecific AZD7789 (FIG. 3B), PD-1-CTLA4 bispecific MEDI5752 (FIG. 3C), ADC hRS7(TROP2)-TOP1i or their combinations (FIGS. 3A-3C).

FIGS. 4A-4B show plots of IFN-7 concentration in supernatants of tumor cells co-cultured with tumor antigen specific T-cells and either PD1-TIGIT bispecific AZD2936 (FIG. 4A), PD-1-TIM3 bispecific AZD7789 (FIG. 4B), PD1-CTLA4 bispecific MEDI5752 (FIG. 4C), ADC hRS7(TROP2)-TOP1i or their combinations (FIGS. 4A-4C).

FIGS. 5A-5C. FIG. 5A shows a schematic of an ADC and Immuno-oncology (IO) agent administration to mice as described in Example 3. FIGS. 5B-5C show plots of monotherapies (FIG. 5B) and combination therapies (FIG. 5C).

FIG. 6 shows spider plots of tumor volume for each mouse treated with each therapy shown in FIGS. 5B-5C and as described in Example 3.

FIGS. 7A-7B. FIG. 7A shows a schematic of an ADC and anti-PD-L1 antibody administration schedule to mice as described in Example 4. FIG. 7B shows spider plots of tumor volume for each mouse treated with each therapy as shown in the figure and described in the example.

FIGS. 8A-8D. FIG. 8A shows a schematic of an ADC and anti-PD-L1 antibody administration to mice as described in Example 5. FIG. 8B is a plot showing the percentage of CD3+ T cells following the treatments indicated as described in the example. FIG. 8C shows plots of the percentages of TIGIT+CD4 T cells and CD8 T cells following the treatments indicated as described in Example 5. FIG. 8D is a Kaplan-Meyer plot of the survival rates of mice in each treatment group.

FIGS. 9A-9B. FIG. 9A shows a schematic of an ADC and IO administration to mice as described in Example 6. FIG. 9B shows spider plots of individual tumor volumes for each mouse treated with each therapy as shown in the figure.

### DETAILED DESCRIPTION

Unless otherwise defined herein, scientific, and technical terms used in the present disclosure shall have the meanings that are commonly understood by one of ordinary skill in the art. Further, unless otherwise required by context, singular terms shall include pluralities and plural terms shall include the singular.

As used herein, “a” or “an” may mean one or more. As used herein, when used in conjunction with the word “comprising,” the words “a” or “an” may mean one or more than one. As used herein, “another” or “a further” may mean at least a second or more.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability (either “greater than” or “less than” the indicated value), depending on the situation. In some aspects, one of skill in the art will understand the level of variability indicated by the term “about,” due to the context in which it is used herein. It should also be understood that use of the term “about” also includes the specifically recited value.

The use of the term “for example” and its corresponding abbreviation “e.g.,” (whether italicized or not) means that the specific terms recited are representative examples and aspects of the disclosure that are not intended to be limited to the specific examples referenced or cited unless explicitly stated otherwise.

Ranges provided herein, of any type, include all values within a particular range described and values about an endpoint for a particular range. As used herein, “between” is a range inclusive of the ends of the range. For example, a number between x and y explicitly includes the numbers x and y, and any numbers that fall within x and y.

The term “treating” or “treatment” refers to administering a compound or pharmaceutical composition to an animal in order to effect an alteration or improvement of a disease, disorder, or condition in the animal. In some aspects, methods of treatment of the disclosure comprise one or more administration step selected from oral, intravenous, intraarterial, intraperitoneal, intramuscular, subcutaneous, rectal, or vaginal, inhalation, topical, or a combination thereof. In a preferable aspect, the administration is intravenous or intraarterial (e.g. by injection or drip), or a combination thereof.

The term “dose” means a specified quantity of a compound or pharmaceutical agent provided in a single administration, or in a specified time period. In some aspects, a dose may be administered in two or more boluses, tablets, or injections. For example, in some aspects, where subcutaneous administration is desired, the desired dose may require a volume not easily accommodated by a single injection. In such aspects, two or more injections may be used to achieve the desired dose. In some aspects, a dose may be administered in two or more injections to minimize injection site reaction in an individual. In other aspects, the compound or pharmaceutical agent is administered by infusion over an extended period of time or continuously. Doses may be stated as the amount of pharmaceutical agent per hour, day, week or month.

The terms “subject”, “individual” and “patient” are used interchangeably herein to refer to a mammalian subject. In one aspect the “subject” is a human, domestic animals, farm animals, sports animals, and zoo animals, e.g., humans, non-human primates, dogs, cats, guinea pigs, rabbits, rats, mice, horses, cattle, etc. In one aspect, the subject is a cynomolgus monkey (Macaca fascicularis). In a preferable aspect, the subject is a human. In methods of the invention, the subject may not have been previously diagnosed as having cancer. Alternatively, the subject may have been previously diagnosed as having cancer. The subject may also be one who exhibits disease risk factors, or one who is asymptomatic for cancer. The subject may also be one who is suffering from or is at risk of developing cancer. Thus, in one aspect, a method of the invention may be used to confirm the presence of cancer in a subject. For example, the subject may previously have been diagnosed with cancer by alternative means. In one aspect, the subject has been previously administered a cancer therapy.

The term “efficacy” means the ability to produce a desired effect.

The term “side effects” means physiological disease and/or conditions attributable to a treatment other than the desired effects. In some aspects, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

Disclosed herein are methods, combinations and kits comprising administration of an antibody-drug conjugate (ADC) together with a bispecific binding protein. The methods combinations and kits can be used in the treatment of cancer in a subject as described herein.

The methods, combinations and kits herein are meant to provide improved cancer treatments by targeting cancer cells in different ways. In some aspects, the ADCs described herein increase the sensitivity of a tumor cell to killing by another mechanism. In some aspects, the ADCs described herein increase the antigenicity of a tumor cell. In some aspects, the bispecific binding proteins target tumor cells for killing by the subject's immune system. In some aspects, the ADC increases the antigenicity of a tumor cell by increasing the availability of one or more cell-surface marker antigens either on the tumor cell itself or on an antigen presenting cell (such as a dendritic cell), while the bispecific binding protein binds the one or more cell-surface marker antigens, triggering an immune response that attacks the tumor cells.

### Combination Methods

In some aspects, the present disclosure provides a method of treating cancer in a human subject in need thereof, comprising administering to the human subject: a) an antibody-drug conjugate (ADC) comprising: i) an antibody or antigen binding fragment thereof which binds to a B7-H4 polypeptide, ii) a cleavable linker, and iii) a cytotoxic agent; and b) a bispecific checkpoint inhibitor. In some aspects, the checkpoint inhibitor is a bispecific binding protein having a first binding domain that specifically binds to Programmed cell death protein 1 (PD-1).

In further aspects, the present disclosure is directed to a method of treating cancer in a human subject in need thereof, comprising administering to the human subject: a) an antibody-drug conjugate (ADC) comprising: i) an antibody or antigen binding fragment thereof, ii) a cleavable linker, and iii) a cytotoxic agent; wherein the cytotoxic agent is a compound of formula I:

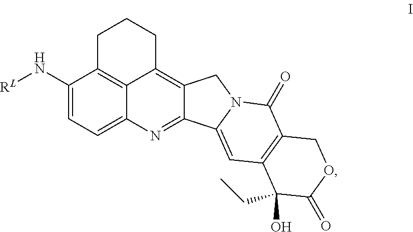

and salts and solvates thereof, wherein R^{L} is the cleavable linker, which is selected from:

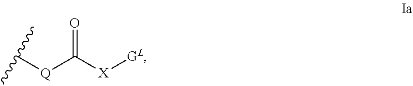

wherein Q is:

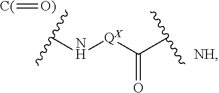

where Q^{X} is such that Q is an amino-acid residue, a dipeptide residue, a tripeptide residue or a tetrapeptide residue; wherein X is:

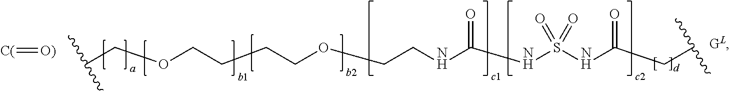

where a=0 to 5, b1=0 to 16, b2=0 to 16, c1=0 or 1, c2=0 or 1, d=0 to 5, at least b1 or b2=0, at least c1 or c2=0; and wherein G^{L} is a linker for connecting to the antibody or antigen binding fragment; and

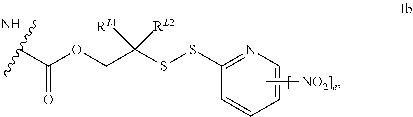

wherein R^{L1} and R^{L2} are independently selected from H and methyl, or together with the carbon atom to which they are bound form a cyclopropylene or cyclobutylene group, and e is 0 or 1; and b) a bispecific checkpoint inhibitor. In some aspects, the checkpoint inhibitor is a bispecific binding protein having a first binding domain that specifically binds to Programmed cell death protein 1 (PD-1).

### Antibody-Drug Conjugates Binding B7-H4

The antibody or antigen binding fragment thereof of the ADC can be used to target a cancer cell. In some aspects, the antibody or antigen binding fragment has high affinity for B7-H4 polypeptide both in vitro an in vivo, and thus may advantageously be used in methods for targeting or detecting a B7-H4 epitope.

B7-H4 (also known as V-set domain-containing T-cell activation inhibitor 1, encoded by the VTCN1 gene) is a transmembrane polypeptide of the B7 family of co-stimulatory proteins. B7-H4 is understood to be expressed on the surface of antigen-presenting cells for interactions with ligands of immune cells (e.g., T-lymphocytes, with CD28 being a potential ligand). B7-H4 has been observed to be highly expressed on cells of various cancer types and is thought to be a tumor-associated antigen. Furthermore, B7-H4 expression is not limited to a particular cancer type, such that it represents a target antigen for treating a broad spectrum of cancer types.

In some aspects, a cancer referred to herein is a cancer characterized by the expression (preferably overexpression) of a B7-H4 molecule. In other words, a cancer referred to herein may comprise a cancerous cell that expresses B7-H4 polypeptide. The cancerous cell may be comprised within a tumor. In some aspects, the B7-H4 polypeptide is comprised within a B7-H4 polypeptide sequence, or a fragment thereof. A “B7-H4 polypeptide” may comprise the full length polypeptide sequence of B7-H4 (e.g., SEQ ID NO:55), or may comprise a fragment of B7-H4 of any length of the full length polypeptide sequence of B7-H4 (e.g., comprising a polypeptide sequence of 5%, 15%, 25%, 35%, 45%, 55%, 65%, 75%, 85% or 95% of the full length polypeptide sequence of B7-H4) which comprises an epitope which can bind (e.g. be bound by) an antibody or antigen binding fragment of the disclosure. The B7-H4 polypeptide may comprise a sequence having 75%, 80%, 85%, 90% or 90% sequence identity to the sequence of SEQ ID NO:55. Preferably, the B7-H4 polypeptide comprises the sequence of SEQ ID NO.: 55.

The term “antibody” covers monoclonal antibodies and fragments thereof (e.g., exhibiting the desired biological activity). In a preferable aspect, an antibody of the present disclosure is a monoclonal antibody. In a more preferable aspect, the antibody is a fully human monoclonal antibody. In some aspects, methods of the disclosure may employ polyclonal antibodies. In particular, an antibody is a protein including at least one or two, heavy (H) chain variable regions (abbreviated herein as VHC), and at least one or two light (L) chain variable regions (abbreviated herein as VLC). The VHC and VLC regions can be further subdivided into regions of hypervariability, termed “complementarity determining regions” (CDR), interspersed with regions that are more conserved, termed “framework regions” (FR). The extent of the framework region and CDRs has been precisely defined. Preferably, each VHC and VLC is composed of three CDRs and four FRs, arranged from amino-terminus to carboxy-terminus in the following order: FR1, CDR1, FR2, CDR2, FR3, CDR3, FR4. The VHC or VLC chain of the antibody can further include all or part of a heavy or light chain constant region. In some aspects, the antibody is a tetramer of two heavy immunoglobulin chains and two light immunoglobulin chains, wherein the heavy and light immunoglobulin chains are interconnected by, e.g., disulfide bonds.

The heavy chain constant region includes three domains, CH1, CH2 and CH3. The light chain constant region is comprised of one domain, CL. The variable region of the heavy and light chains contains a binding domain that interacts with an antigen. The term “antibody” includes intact immunoglobulins of types IgA, IgG, IgE, IgD, IgM (as well as subtypes thereof), wherein the light chains of the immunoglobulin may be of types kappa or lambda. The term antibody, as used herein, also refers to a portion of an antibody that binds to one of the above-mentioned markers, e.g., a molecule in which one or more immunoglobulin chains is not full length, but which binds to a marker. Examples of binding portions encompassed within the term antibody include (i) a Fab fragment, a monovalent fragment consisting of the VLC, VHC, CL and CH1 domains; (ii) a F(ab′)2 fragment, a bivalent fragment comprising two Fab fragments linked by a disulfide bridge at the hinge region; (iii) a Fc fragment consisting of the VHC and CH1 domains; (iv) a Fv fragment consisting of the VLC and VHC domains of a single arm of an antibody, (v) a dAb fragment (Ward et al, Nature 341: 544-546, 1989), which consists of a VHC domain; and (vi) an isolated complementarity determining region (CDR) having sufficient framework to bind, e.g. an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al. (1988) Science 1Al-ATi-Alβ; and Huston et al. (1988) Proc. Natl. Acad. Sci. USA 85: 5879-5883). Such single chain antibodies are also encompassed within the term antibody. These may be obtained using conventional techniques known to those skilled in the art, and the portions are screened for utility in the same manner as are intact antibodies.

In some aspects, the antibody or antigen binding fragment is one or more selected from a murine antibody, a humanized antibody, a chimeric antibody, a monoclonal antibody, a polyclonal antibody, a recombinant antibody, a multispecific antibody, or a combination thereof.

In some aspects, the antigen-binding fragment is one or more selected from a Fv fragment, an Fab fragment, an F(ab′)2 fragment, an Fab′ fragment, a dsFv fragment, an scFv fragment, an sc(Fv)2 fragment, or a combination thereof. In some aspects, the antibody or antigen binding fragment thereof (e.g., mAb) of the disclosure is a scFV.

In some aspects, the antibody or antigen binding fragment thereof can bind to B7-H4 molecules across species, e.g., the antibody or fragment can bind to mouse B7-H4, rat B7-H4, rabbit, human B7-H4 and/or cynomolgus monkey B7-H4. In some aspects, the antibody or fragment can bind to human B7-H4 and cynomolgus monkey B7-H4. In some aspects, the antibody or antigen binding fragment can also bind to mouse B7-H4.

In some aspects, the antibody or antigen binding fragment thereof can specifically bind to B7-H4, e.g., human B7-H4 and cynomolgus monkey B7-H4, but does not specifically bind to human B7-H1, B7-H2, and/or B7-H3.

In some aspects, the antibody or antigen binding fragment thereof of the ADC comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of: SEQ ID NO: 1, SEQ ID NO: 2, SEQ ID NO: 3, SEQ ID NO: 4, SEQ ID NO: 5, and SEQ ID NO: 6, respectively, or a functional variant thereof. An antibody or antigen binding fragment thereof comprising said sequences may be referred to as “ZY0EQD-E02” or “EQD-E02” herein.

In some aspects, the antibody or antigen binding fragment thereof of the ADC comprises a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof. An antibody or antigen binding fragment thereof comprising said sequences may be referred to as “ZY0EPQ-E02” or “EPQ-E02” herein.

In some aspects, the antibody or antigen binding fragment thereof of the ADC comprises a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof. An antibody or antigen binding fragment thereof comprising said sequences may be referred to as “ZY0EOB-F05” or “EOB-F05” herein.

In some aspects, the antibody or antigen binding fragment thereof of the ADC comprises a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof. An antibody or antigen binding fragment thereof comprising said sequences may be referred to as “ZY0EO5-E07” or “E05-E07” herein.

In some aspects, the antibody or antigen binding fragment thereof of the ADC comprises a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof. An antibody or antigen binding fragment thereof comprising said sequences may be referred to as “ZY0EP0-C07” or “EP0-C07” herein.

In some aspects, the antibody or antigen binding fragment thereof of the ADC is a bispecific antibody and comprises a HCDR1, a HCDR2, a HCDR3, a LCDR1, a LCDR2, and a LCDR3 in a first binding arm comprising the amino acid sequence of SEQ ID NO: 116, SEQ ID NO: 117, SEQ ID NO: 118, SEQ ID NO: 119, SEQ ID NO: 120, and SEQ ID NO: 121; and a HCDR1, a HCDR2, a HCDR3, a LCDR1, a LCDR2, and a LCDR3 in a second binding arm comprising the amino acid sequence of SEQ ID NO: 122, SEQ ID NO: 123, SEQ ID NO: 124, SEQ ID NO: 125, SEQ ID NO: 126, and SEQ ID NO: 127.

In some aspects, the antibody or antigen binding fragment thereof having CDRs with the amino acid sequences disclosed above have beneficial advantages, such as high targeting specificity or binding activity toward B7-H4 peptide on the surface of cancer cells.

Additionally or alternatively, an antibody or antigen binding fragment thereof described herein may be described by means of a variable heavy (VH) chain and a variable light (VL) chain thereof.

In some aspects, the antibody or antigen binding fragment thereof of the ADC comprises: a variable heavy (VH) chain and a variable light (VL) chain comprising the amino acid sequence of SEQ ID NO: 45 and SEQ ID NO: 34, respectively, or a functional variant thereof, a VH chain and a VL chain comprising the amino acid sequence of SEQ ID NO: 33 and SEQ ID NO: 34, respectively, or a functional variant thereof; a VH chain and a VL chain comprising the amino acid sequence of SEQ ID NO: 43 and SEQ ID NO: 34, respectively, or a functional variant thereof, a VH chain and a VL chain comprising the amino acid sequence of SEQ ID NO: 46 and SEQ ID NO: 34, respectively, or a functional variant thereof, a VH chain and a VL chain comprising the amino acid sequence of SEQ ID NO: 47 and SEQ ID NO: 34, respectively, or a functional variant thereof; a VH chain and a VL chain comprising the amino acid sequence of SEQ ID NO: 31, and SEQ ID NO: 32, respectively, or a functional variant thereof, a VH chain and a VL chain comprising the amino acid sequence of SEQ ID NO: 35 and SEQ ID NO: 36, respectively, or a functional variant thereof; a VH chain and a VL chain comprising the amino acid sequence of SEQ ID NO: 37 and SEQ ID NO: 38, respectively, or a functional variant thereof, or a VH chain and a VL chain comprising the amino acid sequence of SEQ ID NO: 39 and SEQ ID NO: 40, respectively, or a functional variant thereof.

In some aspects, the antibody or antigen binding fragment thereof of the ADC comprises: (i) a variable heavy chain comprising an amino acid sequence having at least 70%, 75%, 80%, 90%, 95% or 100% sequence identity to the amino acid sequence of SEQ ID NO: 31, 33, 35, 37, or 39, or a functional variant thereof, and (ii) a variable light chain comprising an amino acid sequence having at least 70%, 75%, 80%, 90%, 95% or 100% sequence identity to the amino acid sequence of SEQ ID NO: 32, 34, 36, 38, or 40, or a functional variant thereof.

In some aspects, the antibody or antigen binding fragment thereof of the ADC comprises: (i) a variable heavy chain comprising an amino acid sequence having at least 70%, 75%, 80%, 90%, 95% or 100% sequence identity to the amino acid sequence of SEQ ID NO: 31, 33, 35, 37, 39, 43, 45, 46, or 47, or a functional variant thereof, and (ii) a variable light chain comprising an amino acid sequence having at least 70%, 75%, 80%, 90%, 95% or 100% sequence identity to the amino acid sequence of SEQ ID NO: 32, 34, 36, 38, or 40, or a functional variant thereof.

In some aspects, the antibody or antigen binding fragment thereof of the ADC may comprise: (i) a variable heavy chain comprising an amino acid sequence having at least 70%, 75%, 80%, 90%, 95% or 100% sequence identity to the amino acid sequence SEQ ID NO: 33, or a functional variant thereof; and (ii) a variable light chain comprising an amino acid sequence having at least 70%, 75%, 80%, 90%, 95% or 100% sequence identity to the amino acid sequence of SEQ ID NO: 34, or a functional variant thereof.

In some aspects, the antibody or antigen binding fragment thereof of the ADC may comprise: (i) a variable heavy chain comprising an amino acid sequence having at least 70%, 75%, 80%, 90%, 95% or 100% sequence identity to the amino acid sequence SEQ ID NO: 45, or a functional variant thereof; and (ii) a variable light chain comprising an amino acid sequence having at least 70%, 75%, 80%, 90%, 95% or 100% sequence identity to the amino acid sequence of SEQ ID NO: 34, or a functional variant thereof.

Advantageously, the disclosure has demonstrated that an antibody or antigen binding fragment of the claims may target a broader spectrum of B7-H4 expressing cells when compared with existing (commercially) available antibodies reported to target B7-H4. Thus, not only has the disclosure provided an antibody (or antigen binding fragment) thereof having affinity and specificity for a clinically relevant target, but has also demonstrated a unique advantage (e.g. unexpected technical effect) associated therewith.

In some aspects, antibody or antigen binding fragment thereof described herein is capable of binding to B7-H4 as an integral component of a cancer cell (for example, B7-H4 as an integral component of a cell membrane of a cancer cell).

In some aspects, the antibody or antigen binding fragment thereof of the ADC binds an OVCAR4 cell line. In some aspects, an antibody or antigen binding fragment thereof described herein may bind to an OVCAR4 cell line and/or a CHO cell line (e.g. which may lack an exogenous nucleic acid encoding B7-H4). For example, the antibody or antigen binding fragment thereof binds to a B7-H4 (e.g. a B7-H4 epitope) of an OVCAR4 cell line and/or a CHO cell line (e.g. which may lack an exogenous nucleic acid encoding B7-H4).

In some aspects, the antibody or antigen binding fragment thereof binds to an OVCAR4 cell line and/or CHO cell line (e.g. which may lack an exogenous nucleic acid encoding B7-H4) with higher affinity when compared to one or more antibody selected from E Biosciences 14-5949 anti-Human B7H4 mouse IgG, US biological B0000-35B anti Human B7H4 mouse IgG, R and D systems AF2514 anti-Mouse B7H4 goat IgG1, Sigma SAB2500141 anti B7H4 Goat IgG1, Isotype 1 CAT004 SP06-003, Isotype 2 R and D Normal goat IgG control (AB-108C), AdD serotec MCA2632, Epitomics 2516-1, eBiosciences, 145972-82, eBioscience 145970-85, or a combination thereof. The affinity (e.g. binding affinity) can be measured by any suitable method of measuring binding affinity described herein.

The OVCAR4 cell line is a human ovary carcinoma cell line, the CHO cell line is an epithelial cell line derived from the ovary of the Chinese hamster, and are widely obtainable.

In some aspects, the antibody or antigen binding fragment thereof of the ADC comprises a heavy chain constant region comprising the amino acid sequence of SEQ ID NO: 41. In some aspects, the antibody or antigen binding fragment thereof of the ADC comprises a heavy chain constant region comprising the amino acid sequence of SEQ ID NO: 52. In some aspects, the antibody or antigen binding fragment thereof of the ADC comprises a light chain constant region comprising the amino acid sequence of SEQ ID NO: 42.

In some aspects, the antibody or antigen binding fragment thereof of the ADC comprises a heavy chain comprising the amino acid sequence of SEQ ID NO: 51; and a light chain comprising the amino acid sequence of SEQ ID NO: 44.

In some aspects, the antibody or antigen binding fragment thereof of the ADC comprises a heavy chain comprising the amino acid sequence of SEQ ID NO: 48; and a light chain comprising the amino acid sequence of SEQ ID NO: 44.

In some aspects, the antibody or antigen binding fragment thereof of the ADC is a monoclonal antibody.

In some aspects, the antibody or antigen binding fragment thereof of the ADC is a humanized monoclonal antibody.

### Linkers

In some aspects, the antibody or antigen binding fragment thereof is linked to the cytotoxic agent by a linker. In some aspects, the antibody or antigen binding fragment thereof is conjugated to the cytotoxic agent by a linker. As used herein, “conjugated” means linked via a covalent or ionic bond. In some aspects, the linker is attached (e.g. conjugated) in a cleavable manner to an amino reside, for example, an amino acid of the antibody or antigen binding fragment of the ADC. In some aspects, the cleavable linker of the ADC is an mp-PEG8-val-ala linker.

In some aspects, the linker is represented by R^{L}, and is preferably selected from: (Ia):

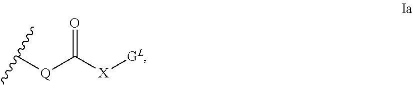

where
Q is linked to the cytotoxic agent and has a formula

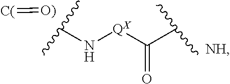

where Q^{X} is such that Q is an amino-acid residue, a dipeptide residue, a tripeptide residue or a tetrapeptide residue;
X is:

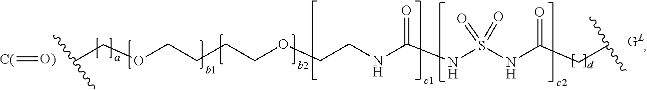

where a=0 to 5, b1=0 to 16, b2=0 to 16, c1=0 or 1, c2=0 or 1, d=0 to 5, wherein at least b1 or b2=0 (i.e., only one of b1 and b2 may not be 0) and at least c1 or c2=0 (i.e., only one of c1 and c2 may not be 0);
G^{L} is a linker for connecting to an antibody or antigen binding fragment thereof described herein; and (Ib):

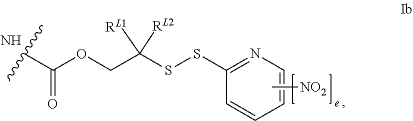

where
R^{L1} and R^{L2} are independently selected from H and methyl, or together with the carbon atom to which they are bound form a cyclopropylene or cyclobutylene group; and e is 0 or 1.

In some aspects, Q is an amino acid residue. The amino acid may be a natural amino acid or a non-natural amino acid. For example, Q may be selected from: Phe, Lys, Val, Ala, Cit, Leu, Ile, Arg, and Trp, where Cit is citrulline.

In some aspects, Q comprises a dipeptide residue. The amino acids in the dipeptide may be any combination of natural amino acids and non-natural amino acids. In some aspects, the dipeptide comprises natural amino acids. Where the linker is a cathepsin labile linker, the dipeptide is the site of action for cathepsin-mediated cleavage. The dipeptide then is a recognition site for cathepsin.

In some aspects, Q is selected from: ^{NH}-Phe-Lys-^{C═O}, ^{NH}-Val-Ala-^{C═O}, ^{NH}-Val-Lys-^{C═O}, ^{NH}-Ala-Lys-^{C═O}, ^{NH}-Val-Cit-^{C═O}, ^{NH}-Phe-Cit-^{C═O}, ^{NH}-Leu-Cit-^{C═O}, NH-Ile-Cit-^{C═O}, ^{NH}-Phe-Arg-^{C═O}, ^{NH}-Trp-Cit-^{C═O}, and ^{NH}-Gly-Val-^{C═O}; where Cit is citrulline. Preferably, Q is selected from: NH-Phe-Lys-^{C═O}, ^{NH}-Val-Ala-^{C═O}, ^{NH}-Val-Lys-^{C═O}, ^{NH}-Ala-Lys-^{C═O}, and ^{NH}-Val-Cit-^{C═O}. More preferably, Q is selected from ^{NH}-Phe-Lys-^{C═O}, ^{NH}-Val-Cit-^{C═O} and ^{NH}-Val-Ala-^{C═O}.

Other suitable dipeptide combinations include: ^{NH}-Gly-Gly-^{C═O}, ^{NH}-Gly-Val-^{C═O}, ^{NH}-Pro-Pro-^{C═O}, and ^{NH}-Val-Glu-^{C═O}. Other dipeptide combinations may be used, including those described by Dubowchik et al., Bioconjugate Chemistry, 2002, 13, 855-869, which is hereby incorporated herein by reference.

In some aspects, Q is a tripeptide residue. The amino acids in the tripeptide may be any combination of natural amino acids and non-natural amino acids. In some aspects, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are: ^{NH}-Glu-Val-Ala-^{C═O}, ^{NH}-Glu-Val-Cit-^{C═O}, ^{NH}-αGlu-Val-Ala-^{C═O}, and ^{NH}-αGlu-Val-Cit-^{C═O}.

In some aspects, Q is a tetrapeptide residue. The amino acids in the tetrapeptide may be any combination of natural amino acids and non-natural amino acids. In some aspects, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are: ^{NH}-Gly-Gly-Phe-Gly ^{C═O}; and ^{NH}-Gly-Phe-Gly-Gly^{C═O}. Preferably, the tetrapeptide is: ^{NH}-Gly-Gly-Phe-Gly^{C═O}.

In the above representations of peptide residues, ^{NH}— represents the N-terminus, and —^{C═O}, represents the C-terminus of the residue. The C-terminus binds to, for example, the NH of the cytotoxic agent.

Glu represents the residue of glutamic acid, i.e.:

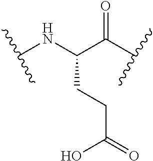 [SMILES: *N[C@@H](CCC(=O)O)C(*)=O]

αGlu represents the residue of glutamic acid when bound via the α-chain, i.e.:

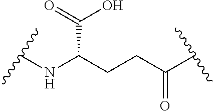 [SMILES: *N[C@@H](CCC(*)=O)C(=O)O]

In some aspects, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin. In some aspects, protecting groups for the side chains of amino acids are well known and are described in the Novabiochem Catalog, and as described above.

In some aspects of X, a may be 0, 1, 2, 3, 4 or 5. Preferably, a is 0 to 3. More preferably, a is 0 or 1. More preferably, a is 0. In some aspects, b1 may be 0, 1, 2, 3, 4, 5, 6, 7, 8, 9, 10, 11, 12, 13, 14, 15 or 16. Preferably, b1 is 0 to 12. More preferably, b1 is 0 to 8, and may be 0, 2, 3, 4, 5 or 8. In some aspects, b2 may be 0, 1, 2, 3, 4, 5, 6, 7, 8, 9, 10, 11, 12, 13, 14, 15 or 16. Preferably, b2 is 0 to 12. More preferably, b2 is 0 to 8, and may be 0, 2, 3, 4, 5 or 8. In some aspects, c1 may be 0 or 1. In some aspects, c2 may be 0 or 1. Preferably, only one of c1 and c2 may not be 0. In some aspects, d may be 0, 1, 2, 3, 4 or 5. Preferably, d is 0 to 3. More preferably, d is 1 or 2. In an aspect, d is 2. In an aspect, d is 5.

In some aspects of X, a is 0, b1 is 0, c1 is 1, c2 is 0 and d is 2, and b2 may be from 0 to 8. In some aspects, b2 is 0, 2, 3, 4, 5 or 8. In some aspects of X, a is 1, b2 is 0, c1 is 0, c2 is 0 and d is 0, and b1 may be from 0 to 8. In some aspects, b1 is 0, 2, 3, 4, 5 or 8. In some aspects of X, a is 0, b1 is 0, c1 is 0, c2 is 0 and d is 1, and b2 may be from 0 to 8. In some aspects, b2 is 0, 2, 3, 4, 5 or 8. In some aspects of X, b1 is 0, b2 is 0, c1 is 0, c2 is 0 and one of a and d is 0. The other of a and d is from 1 to 5. In some aspects, the other of a and d is 1. In some aspects, the other of a and d is 5. In some aspects of X, a is 1, b2 is 0, c1 is 0, c2 is 1, d is 2, and b1 may be from 0 to 8. In some aspects, b2 is 0, 2, 3, 4, 5 or 8.

As described above R^{L1} and R^{L2} may be independently selected from H and methyl, or together with the carbon atom to which they are bound form a cyclopropylene or cyclobutylene group.

In some aspects, both R^{L1} and R^{L2} are H. In some aspects, R^{L1} is H and R^{L2} is methyl. In some aspects, both R^{L1} and R^{L2} are methyl. In some aspects, R^{L1} and R^{L2} together with the carbon atom to which they are bound form a cyclopropylene group. In some aspects, R^{L1} and R^{L2} together with the carbon atom to which they are bound form a cyclobutylene group.

In the formula Ib, In some aspects, e is 0. In some aspects, e is 1 and the nitro group may be in any available position of the ring. In some aspects, it is in the ortho position. In some aspects, it is in the para position.

In some aspects, G^{L} may be selected from:

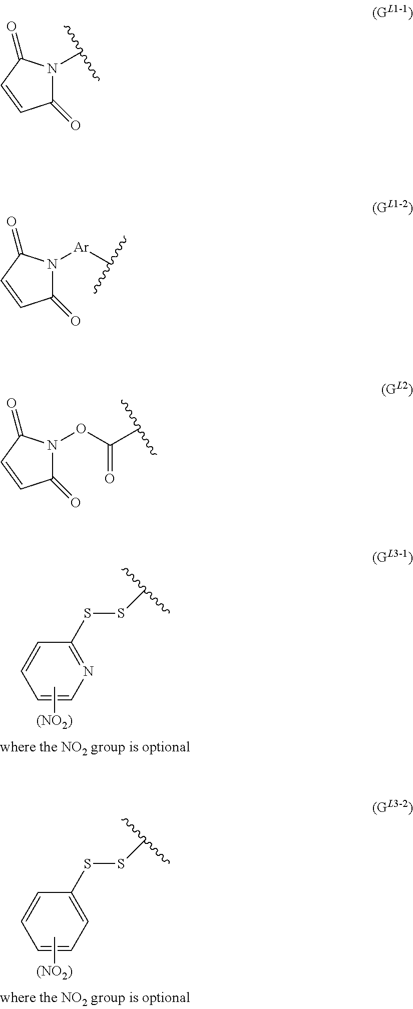

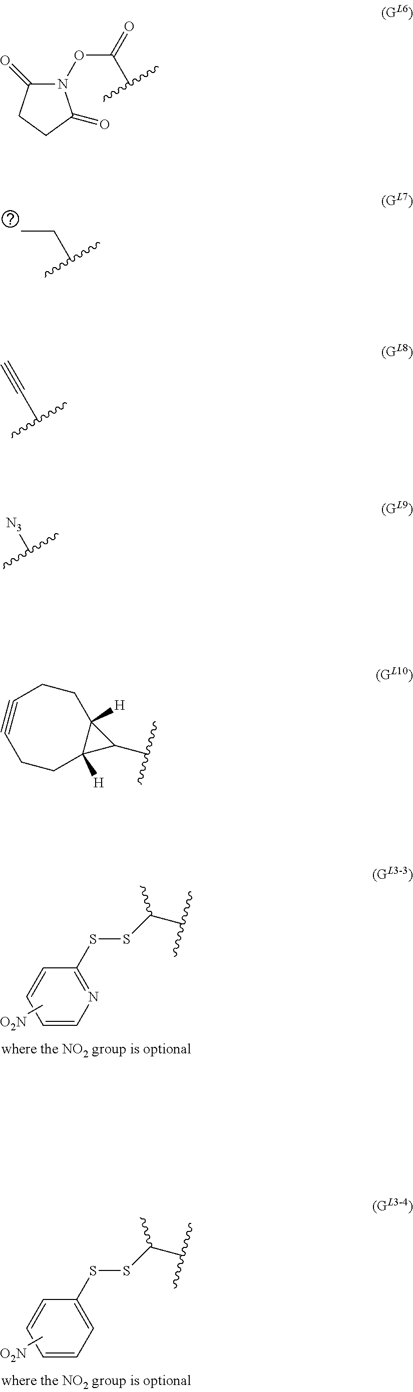

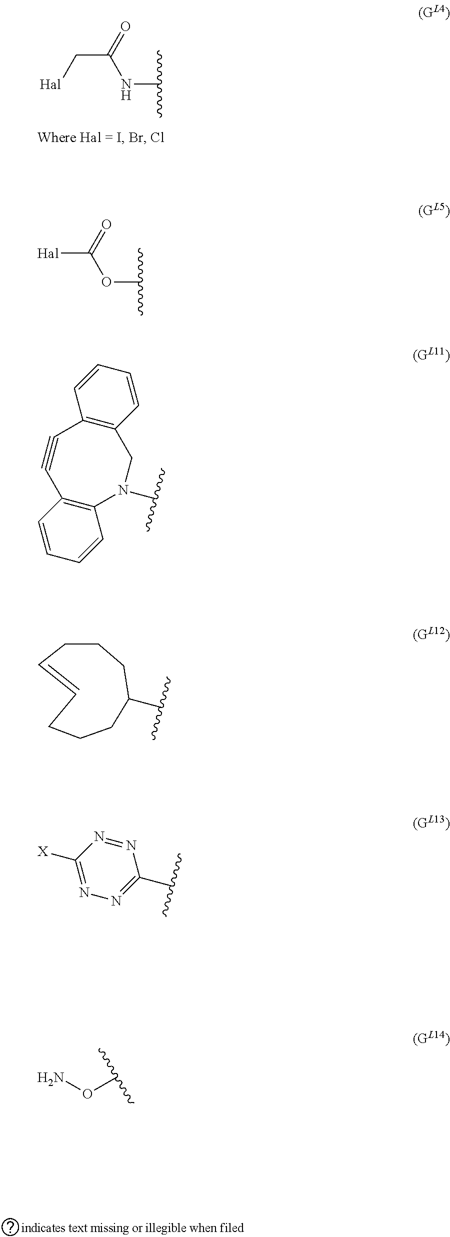

where Ar represents a C₅₋₆ arylene group, e.g., phenylene, and X represents C₁₋₄ alkyl.

In some aspects, G^{L} is selected from G^{L1-1} and G^{L1-2}. In one aspect, G^{L} is G^{L1-1}.

In some aspects, R^{L} is selected from:

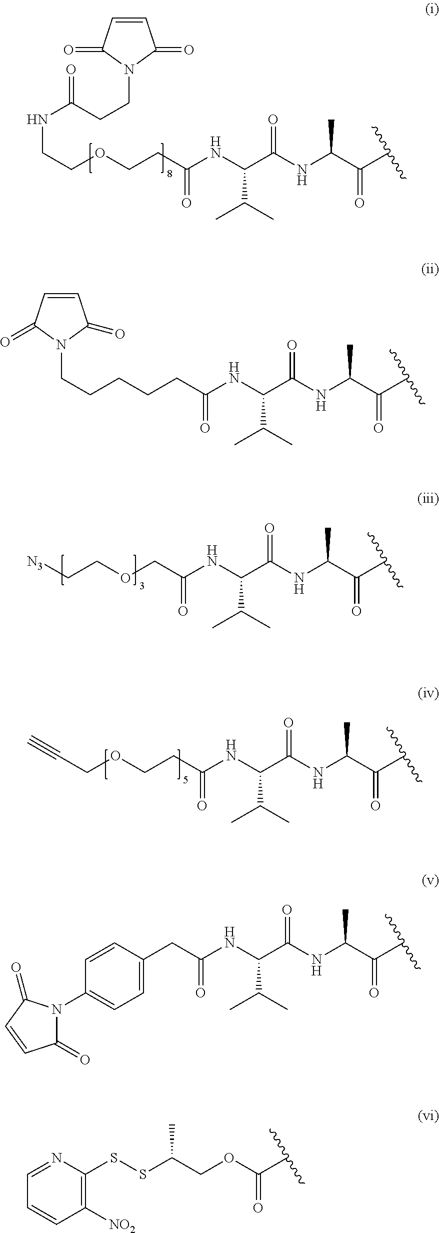

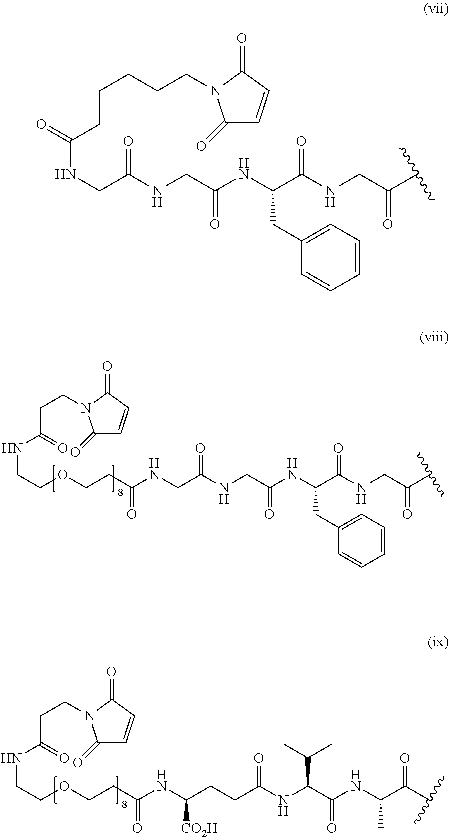

In some aspects, the linker is represented by R^{LL}, and is preferably selected from:

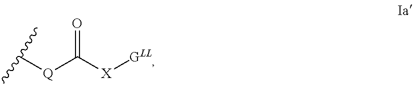

where
Q and X are as defined above and G^{LL} is a linker connected to an antibody or antigen binding fragment thereof described herein (e.g., the Ligand Unit); and (Ib′):

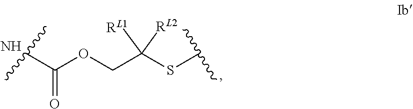

where
R^{L1} and R^{L2} are as defined above; and p is an integer of from 1 to 20.

In some aspects, G^{LL} may be selected from:

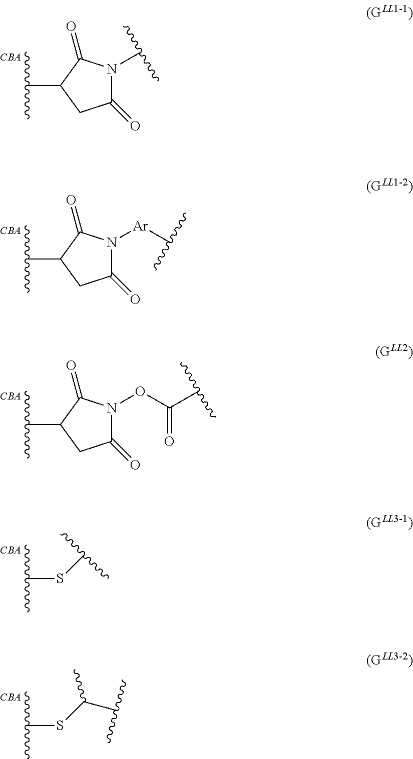

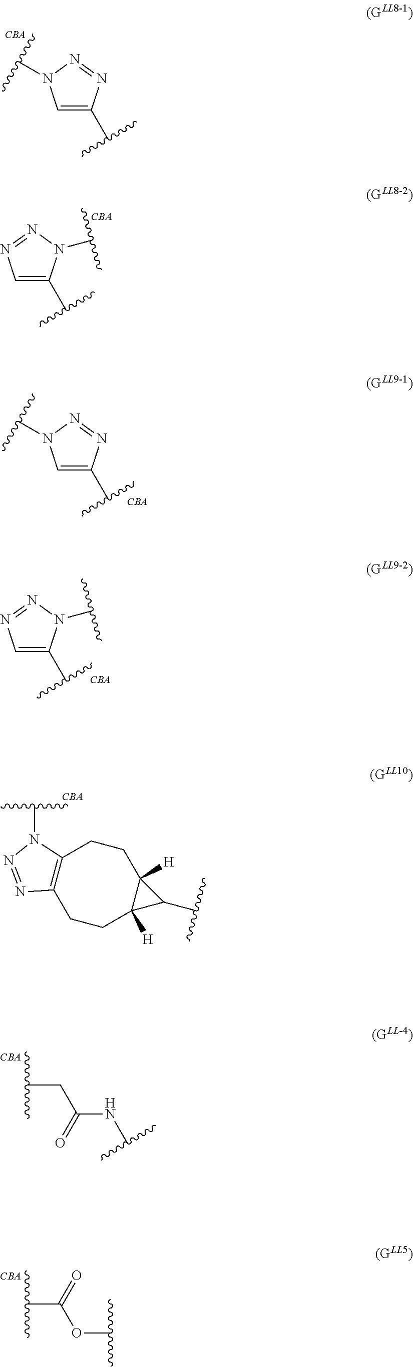

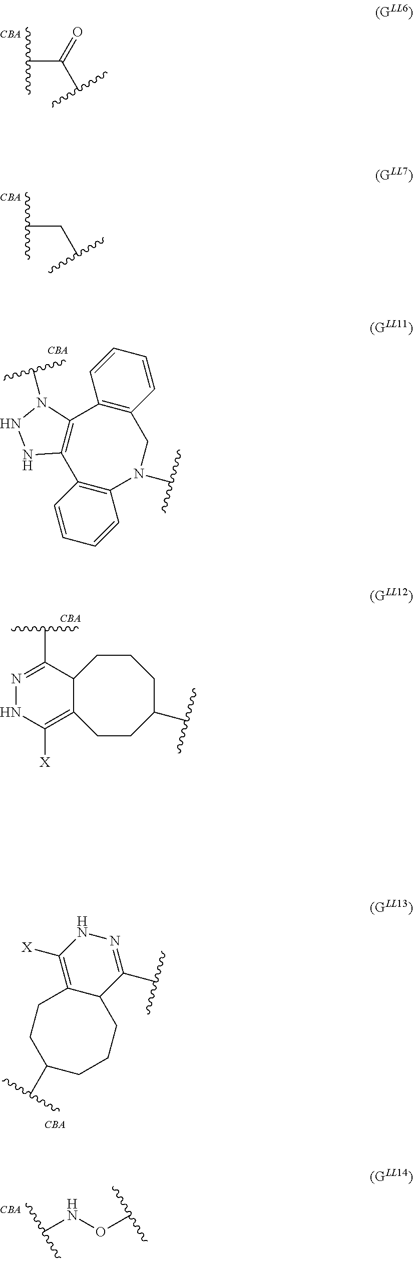

where Ar represents a C₅₋₆ arylene group, e.g., phenylene and X represents C₁₋₄ alkyl.

In some aspects, G^{LL} is selected from G^{LL1-1} and G^{LL1-2}. In one aspect, G^{LL} is G^{LL1-1}.

In some aspects, R^{LL} is a group derived from the R^{L} groups above.

In some aspects where compounds described herein are provided in a single enantiomer or in an enantiomerically enriched form, the enantiomerically enriched form has an enantiomeric ratio greater than 60:40, 70:30; 80:20 or 90:10. In some aspects, the enantiomeric ratio is greater than 95:5, 97:3 or 99:1.

### Drug Loading

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

Typically, fewer than the theoretical maximum of drug moieties are conjugated to an antibody during a conjugation reaction. An antibody may contain, for example, many lysine residues that do not react with the Drug Linker. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of Drug Linker relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Doman et al (2009) Blood 114(13):2721-2729; U.S. Pat. Nos. 7,521,541; 7,723,485; WO2009/052249). The engineered cysteine thiols may react with Drug Linkers of the present invention which have thiol-reactive, electrophilic groups such as maleimide or alpha-halo amides to form ADC with cysteine engineered antibodies. The location of the drug unit can thus be designed, controlled, and known. The drug loading can be controlled since the engineered cysteine thiol groups typically react with drug-linker reagents in high yield. Engineering an IgG antibody to introduce a cysteine amino acid by substitution at a single site on the heavy or light chain gives two new cysteines on the symmetrical antibody.

### Cytotoxic Agent

The cytotoxic agent of the ADC may be referred to herein as an “agent” or “active agent.” In some aspects, the cytotoxic agent is a drug. In some aspects, the cytotoxic agent of the ADC is a topoisomerase inhibitor, tubulysin derivative, a pyrrolobenzodiazepine, or a combination thereof.

In some aspects, the topoisomerase inhibitor is a compound of formula A:

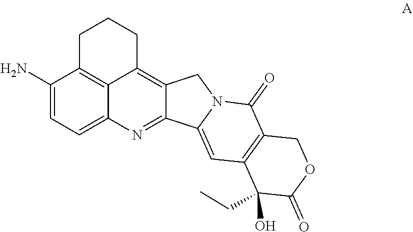 [SMILES: CC[C@@]1(O)C(=O)OCc2c1cc1n(c2=O)Cc2c-1nc1ccc(N)c3c1c2CCC3]

as a single enantiomer or in an enantiomerically enriched form.

In some aspects, the disclosure provides a conjugate comprising the following topoisomerase inhibitor derivative (A*, the Drug Unit):

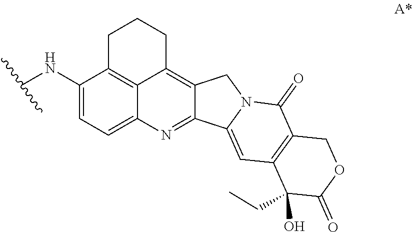 [SMILES: *Nc1ccc2nc3c(c4c2c1CCC4)Cn1c-3cc2c(c1=O)COC(=O)[C@]2(O)CC]

The present disclosure provides the cytotoxic agent (Dug Unit) of the ADC, which is connected to a Ligand Unit of the ADC via the linker (linking unit) described above. The Ligand Unit is preferably an antibody or antigen binding fragment thereof. In some aspects, the present disclosure also provides A* with the linker attached, and intermediates for their synthesis, as well as the released warhead.

In some aspects, the cytotoxic agent is a topoisomerase inhibitor and wherein the cleavable linker and cytotoxic agent of the ADC together comprise a compound of formula I:

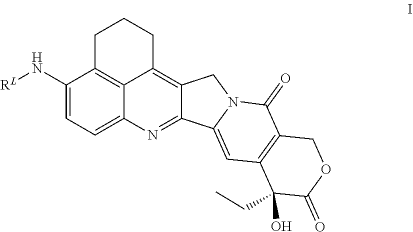

and salts and solvates thereof. R^{L} is the cleavable linker described above.

In some aspects, the ADC is of formula IV:

L-(D^{L})ₚ (IV)
or a pharmaceutically acceptable salt or solvate thereof,
wherein L is the i) antibody or antigen binding fragment thereof, D^{L} is a drug linker unit comprising the ii) cleavable linker and the iii) cytotoxic agent, and D^{L} is of formula III:

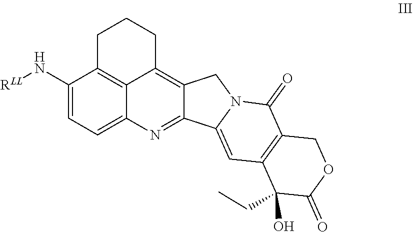

wherein the cleavable linker R^{LL} is as described above.

In some aspects, the drug loading (p) of the cytotoxic agent to the antibody or antibody binding fragment is an integer from 1 to about 10.

In some aspects, the topoisomerase inhibitor is a compound with the formula VI:

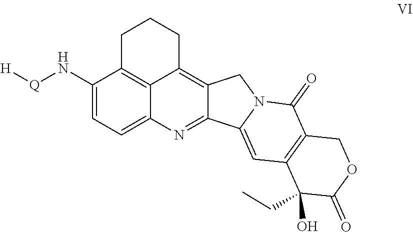

where Q as described above in related to the linker R^{L}.

In some aspects, the linker and cytotoxic agent together comprise the following compound:

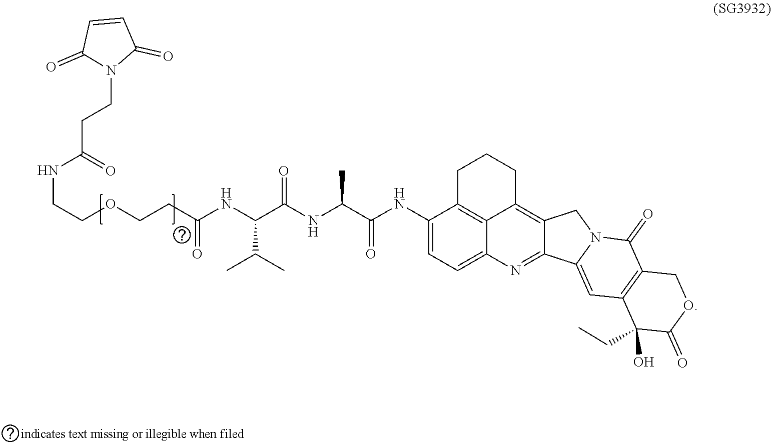

In some aspects, the linker and cytotoxic agent together one of the following compounds:

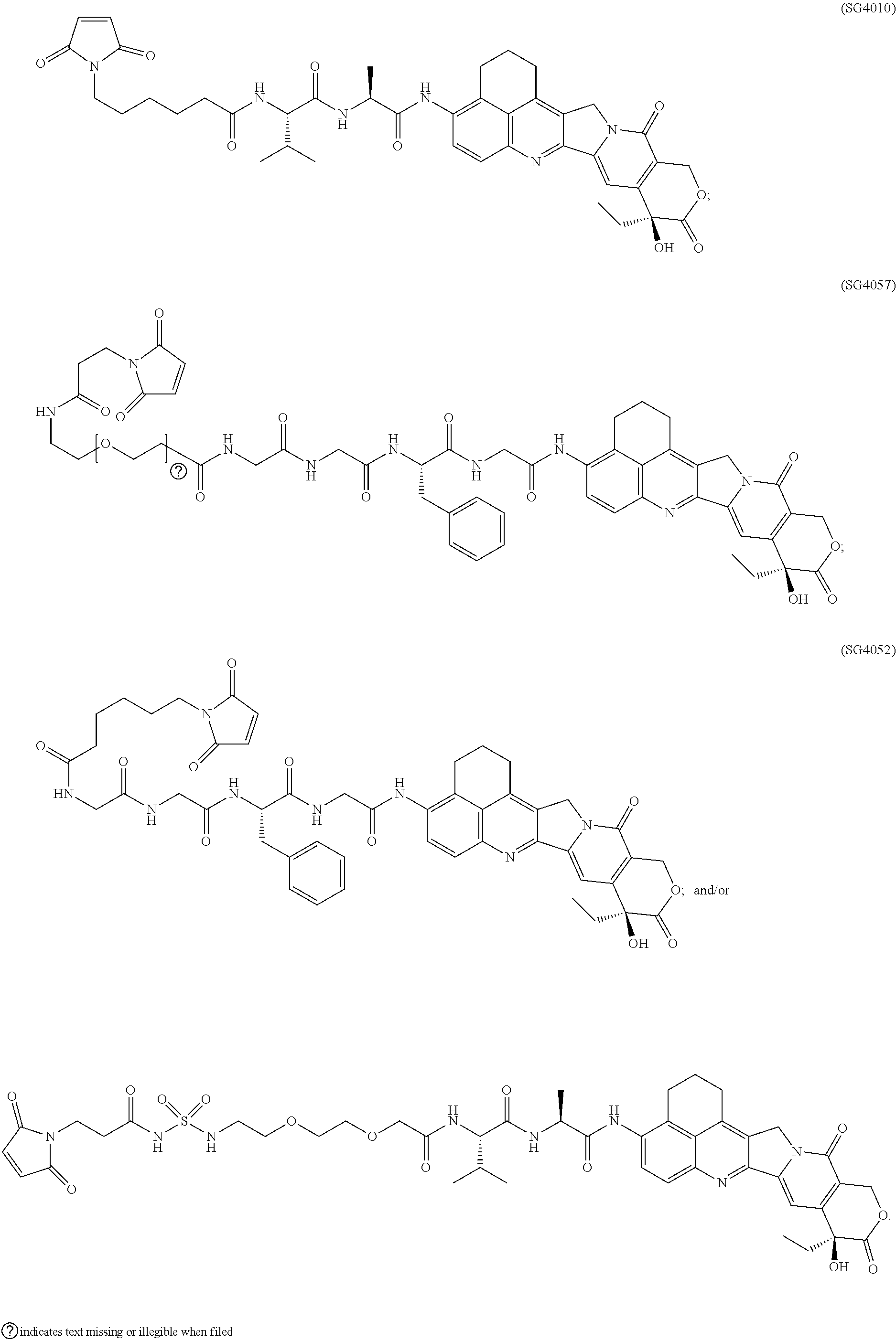

The cytotoxic agent is typically linked to, or “loaded onto” the antibody or antigen-binding fragment. The agent loading (p) is the average number of agent(s) per antibody or antigen-binding fragment (e.g., the Ligand Unit).

The average number of agents per antibody (or antigen-binding fragment) in preparations of ADCs from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). In some instances, separation, purification, and characterization of homogeneous ADC, where p is a certain value from ADC with other drug loadings, may be achieved by means such as reverse phase HPLC or electrophoresis. Such techniques are also applicable to other types of conjugates.

Cysteine amino acids may be engineered at reactive sites in an antibody (or antigen-binding fragment thereof) and which preferably do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249). The engineered cysteine thiols may react with a linker within an agent (e.g., of formula I below) which may have thiol-reactive, electrophilic groups such as maleimide or alpha-halo amides to form ADC with cysteine engineered antibodies. The location of the drug unit can thus be designed, controlled, and known. The drug loading can be controlled since the engineered cysteine thiol groups typically react with drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody or antigen binding fragment thereof reacts with an agent, then the resulting product may be a mixture of ADC compounds with a distribution of agent units attached to an antibody, e.g., 1, 2, 3, etc. Liquid chromatography methods such as polymeric reverse phase (PLRP) and hydrophobic interaction (HIC) may separate compounds in the mixture by agent loading value. Preparations of ADC with a single agent loading value (p) may be isolated.

Thus, the antibody-drug conjugate compositions of the disclosure may include mixtures of antibody-drug conjugates where the antibody or antigen binding fragment thereof has one or more agent moieties and where the agent moieties may be attached to the antibody or antigen binding fragment thereof at various amino acid residues.

In some aspects, the average number of agents per antibody (or antigen-binding fragment thereof) is in the range 1 to 20. In some aspects the range is selected from 1 to 10, 2 to 10, 2 to 8, 2 to 6, and 4 to 10. In some aspects, there is one agent per antibody (or antigen-binding fragment thereof). In some aspects, the number of agents per antibody (or antigen-binding fragment thereof) can be expressed as a ratio of agent (i.e., drug) to antibody. This ratio is referred to as the Drug to Antibody Ratio (DAR). The DAR is the average number of drugs (i.e., agents) linked to each antibody. In some aspects of the present disclosure, the DAR is in the range 1 to 20. In some aspects the range of DAR is selected from 1 to 10, 2 to 10, 2 to 8, 2 to 6, and 4 to 10. In some aspects, the DAR is between about 1 and about 8. In a particular aspect of the present disclosure, the DAR is about 8. In a particular aspect of the present disclosure, the DAR is 8.

In some aspects, the ADC is AZD8205. AZD 8205 is an anti-B7H4 Ab conjugated to topoisomerase inhibitor (TOPO) warhead. In some aspects, the AZD 8205 is targeted to ovarian cancer and cholangiocarcinoma (CCA). In some aspects, the AZD 8205 is targeted to triple negative breast cancer (TNBC). In some aspects, the ADC is an ADC described in WO Publication No. WO2022/053650, the disclosure of which is hereby incorporated by reference in its entirety. In some aspects, the antibody has specific CDRs and binding specificity. In some aspects the antibody sequences are found in WO Publication No. WO2022/053650, and this antibody or antigen binding fragment thereof includes HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, and/or VH chain and a VL chain comprising the amino acid sequence of SEQ ID NO: 45 and SEQ ID NO: 34 (germlined), and/or a heavy chain comprising the amino acid sequence of SEQ ID NO: 51, and a light chain comprising the amino acid sequence of SEQ ID NO: 44 (germlined). In some aspects, the ADC is named E02-GL-SG3932.

### Bispecific Binding Proteins

The term “bispecific binding protein” means a protein that has binding specificities for at least two independent antigens (or targets) or different epitopes within the same antigen. Exemplary bispecific binding proteins may bind to two different epitopes of a target, or may bind two different targets. Other such binding proteins may combine a first target binding site with a second binding site for another target. In some aspects, the bispecific binding protein is a bispecific antibody. In some aspects, the bispecific binding protein is a bispecific checkpoint inhibitor.

In the present disclosure, the term “bispecific checkpoint inhibitor” refers to checkpoint inhibitors that are bispecific. As used herein, bispecific checkpoint inhibitors have binding specificities for at least two independent antigens (or targets) or different epitopes within the same antigen. Exemplary bispecific binding proteins may bind to two different epitopes of a target, or may bind two different targets. Other such binding proteins may combine a first target binding site with a second binding site for another target. Checkpoint inhibitors (or “immuno-oncology (IO) agents”) are agents that inhibit the immune suppression system and activate antitumor immunity (Menon S. et al., Cancers (2016) 8, 106; Pardoll D M., Nat Rev Cancer (2012) 12, 252-264; Wolchok J D., Cell (2015) 162, 937). Targets for immune mediated therapy in oncology include PD-1 (programmed cell death protein-1) and CTLA-4 (cytotoxic T-lymphocyte-associated protein 4). TIGIT (T cell immunoreceptor with Ig and ITIM domains), an immune receptor present on some T cells and natural killer cells (NKs), is another target, along with Leukemia Inhibitory Factor (LIF). Examples of known immune checkpoint inhibitors include anti-PD-1 antibodies nivolumab (WO 2006/121168) and pembrolizumab (WO 2008/156712); anti-PD-L1 antibodies atezolizumab (WO 2010/077634), durvalumab (WO 2011/066389), and avelumab (WO 2013/079174); and anti-CTLA-4 antibodies ipilimumab (WO 2001/014424) and tremelimumab (WO 2000/037504), and anti-LIF antibodies such as h5D8 (U.S. Pat. No. 10,583,191), each of which are hereby incorporated by reference in their entirety. In some aspects, the bispecific antibodies provide additive and/or synergistic therapeutic effects derived from targeting two antigens simultaneously, with the administration of a single manufactured molecule.

In some aspects, the antibodies provided herein are monovalent bispecific antibodies (MBab). The monovalent bispecific antibody scaffolds described herein provide a superior platform for the generation of bispecific antibodies that fulfill all the benefits associated with bispecific antibodies while reducing the potential therapeutic risks mentioned above due to their monovalent nature. Furthermore, the MBabs provided herein are readily expressed, stable, and are likely to have low immunogenicity. As used herein, the term “monovalent bispecific,” which may be abbreviated “MBab,” refers to bispecific antibodies, where each arm can specifically bind to a different target antigen, and for a given pair of different target antigens (A and B), the MBab can bind to one of each. In some aspects, monovalent bispecific antibodies can specifically bind to two independent antigens (or targets) or two independent epitopes on the same antigen. Typically, monovalent bispecific antibodies comprise two different variable regions. In some aspects, the binding affinity for the two independent antigens is about the same. In some aspects, the binding affinities for the two independent antigens are different.

In some aspects, the bispecific binding protein comprises: (a) the first binding domain that specifically binds to PD-1; and (b) a second binding domain that specifically binds to T cell immunoreceptor with Ig and ITIM domains (TIGIT), T-cell immunoglobulin and mucin domain containing protein-3 (TIM-3) or cytotoxic T-lymphocyte-associated antigent-4 (CTLA 4).

### PD-1 and PD-L1 Binding Domains

The term Programmed cell death protein 1 (PD-1) refers to an approximately 31 kD type I membrane protein that is member of the extended CD28/CTLA4 family of T cell regulators (see Ishida et al., “Induced Expression of PD-1, A Novel Member of the Immunoglobulin Gene Superfamily, Upon Programmed Cell Death,” EMBO J. 11: 3887-95 (1992)). PD-1 is expressed on activated T cells, B cells, and monocytes and at low levels in natural killer (NK) T cells. Programmed death ligand 1 (PD-L1) is a ligand of PD-1. When PD-L1 binds PD-1 on T cells, it prevents the T cells from killing the cells displaying PD-L1 (see Han et al., “PD-1/PD-L1 pathway: current researches in cancer,” Am J. Cancer Res., 10(3):727-742 (2020)). PD-L1 is an approximate 33 kD type 1 transmembrane glycoprotein that is a member of the B7 family (see Sanmamed et al. “Inducible expression of B7-H1 (PD-L1) and its selective role in tumor site immune modulation,”. Cancer J. 20:256-261 (2014)). Antagonistic inhibition of the PD-1/PD-L1 interaction increases T cell activation, enhancing recognition and elimination of tumor cells by the host immune system. Such antagonistic inhibition of the PD-1/PD-L1 interaction can be achieved using agents that bind to either PD-1 or PD-L1, blocking the interaction. The amino acid sequence of the human PD-1 protein is provided as UNIPROT ID no. Q15116, while the amino acid sequence of the human PD-L1 is provided as UNIPROT ID no. Q9NZQ7.

Programmed Death Ligand 1 (PD-L1) is also part of a complex system of receptors and ligands that are involved in controlling T-cell activation. In normal tissue, PD-L1 is expressed on T cells, B cells, dendritic cells, macrophages, mesenchymal stem cells, bone marrow-derived mast cells, as well as various non-hematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1-dependent immunosuppressive effects and enhance the cytotoxic activity of antitumor T cells in vitro. Durvalumab is a human monoclonal antibody directed against human PD-L1 that is capable of blocking the binding of PD-L1 to both the PD-1 and CD80 receptors. The use of anti-PD-L1 antibodies to treat infections and tumors and enhance an adaptive immune response has been proposed (see, U.S. Pat. Nos. 8,779,108 and 9,493,565 incorporated herein by reference in their entirety).

As used herein, the term “MEDI5752” refers to an anti-PD-1/CTLA-4 bispecific antibody that comprises the light chain of SEQ ID NO: 45 and the heavy chain of SEQ ID NO: 46 (PD-1) and the light chain of SEQ ID NO: 47 and the heavy chain of SEQ ID NO: 48 (CTLA-4). MEDI5752 is disclosed in U.S. Pat. No. 10,457,732, which is incorporated by reference herein in its entirety.

In some aspects, the first binding domain comprises a heavy chain variable domain comprising a HCDR1 having the amino acid sequence of SEQ ID NO: 56, a HCDR2 having the amino acid sequence of SEQ ID NO: 57, and a HCDR3 having the amino acid sequence of SEQ ID NO: 58, and a light chain variable domain comprising a LCDR1 having the amino acid sequence of SEQ ID NO: 59, a LCDR2 having the amino acid sequence of SEQ ID NO: 60 and a LCDR3 having the amino acid sequence of SEQ ID NO: 61.

In some aspects, the first binding domain that specifically binds to PD-1 comprises a heavy chain variable domain having the amino acid sequence of SEQ ID NO: 62 and a light chain variable domain having the amino acid sequence of SEQ ID NO: 64.

In some aspects, the first binding domain that specifically binds to PD-1 comprises a heavy chain variable domain having an amino acid sequence that is at least 90, 91, 92, 93, 94, 95, 96, 97, 98, or 99% identical to the amino acid sequence of SEQ ID NO: 62. In some aspects, the first binding domain that specifically binds to PD-1 comprises a light chain variable domain having an amino acid sequence that is at least 90, 91, 92, 93, 94, 95, 96, 97, 98, or 99% identical to the amino acid sequence of SEQ ID NO: 64.

In some aspects, the first binding domain that specifically binds to PD-1 comprises a heavy chain having the amino acid sequence of SEQ ID NO: 63 and a light chain having the amino acid sequence of SEQ ID NO: 65.

In some aspects, the first binding domain that specifically binds to PD-1 comprises a heavy chain having an amino acid sequence that is at least 90, 91, 92, 93, 94, 95, 96, 97, 98, or 99% identical to the amino acid sequence of SEQ ID NO: 63. In some aspects, the first binding domain that specifically binds to PD-1 comprises a light chain having an amino acid sequence that is at least 90, 91, 92, 93, 94, 95, 96, 97, 98, or 99% identical to the amino acid sequence of SEQ ID NO: 65.

In some aspects, the first binding domain that specifically binds to PD-1 comprises a heavy chain variable domain that is encoded by the nucleic acid sequence of SEQ ID NO: 76 and a light chain variable domain is encoded by the nucleic acid sequence of SEQ ID NO: 78.

In some aspects, the first binding domain that specifically binds to PD-1 comprises a heavy chain variable domain that is encoded by a nucleic acid sequence that is at least 90, 91, 92, 93, 94, 95, 96, 97, 98, or 99% identical to SEQ ID NO: 76 and a light chain variable domain is encoded by a nucleic acid sequence that is at least 90, 91, 92, 93, 94, 95, 96, 97, 98, or 99% identical to SEQ ID NO: 78.

In some aspects, the first binding domain that specifically binds to PD-1 comprises a heavy chain is encoded by the nucleic acid sequence of SEQ ID NO: 77 and a light chain is encoded by the nucleic acid sequence of SEQ ID NO: 79.

In some aspects, the first binding domain that specifically binds to PD-1 comprises a heavy chain is encoded by a nucleic acid sequence that is at least 90, 91, 92, 93, 94, 95, 96, 97, 98, or 99% identical to SEQ ID NO: 77 and a light chain is encoded by a nucleic acid sequence that is at least 90, 91, 92, 93, 94, 95, 96, 97, 98, or 99% identical to SEQ ID NO: 79.

### TIGIT Binding Domains

The term T cell immunoreceptor with Ig and ITIM domains (TIGIT) refers to an immune receptor present on some T cells and Natural Killer Cells (NK). TIGIT is upregulated by immune cells, including activated T cells, natural killer cells, and regulatory T cells.

In some aspects, the second binding domain that specifically binds to TIGIT. In some aspects, the second binding domain comprises a heavy chain variable domain comprising a HCDR1 having the amino acid sequence of SEQ ID NO: 66, a HCDR2 having the amino acid sequence of SEQ ID NO: 67, and a HCDR3 having the amino acid sequence of SEQ ID NO: 68, and a light chain variable domain comprising a LCDR1 having the amino acid sequence of SEQ ID NO: 69, a LCDR2 having the amino acid sequence of SEQ ID NO: 70, and a LCDR3 having the amino acid sequence of SEQ ID NO: 71.

In some aspects, the second binding domain that specifically binds TIGIT comprises a heavy chain variable domain having the amino acid sequence of SEQ ID NO: 72 and a light chain variable domain having the amino acid sequence of SEQ ID NO: 74.

In some aspects, the second binding domain that specifically binds to TIGIT comprises a heavy chain variable domain having an amino acid sequence that is at least 90, 91, 92, 93, 94, 95, 96, 97, 98, or 99% identical to the amino acid sequence of SEQ ID NO: 72. In some aspects, the second binding domain that specifically binds to TIGIT comprises a light chain variable domain having an amino acid sequence that is at least 90, 91, 92, 93, 94, 95, 96, 97, 98, or 99% identical to the amino acid sequence of SEQ ID NO: 74.

In some aspects, the second binding domain that specifically binds to TIGIT comprises a heavy chain having the amino acid sequence of SEQ ID NO: 73 and a light chain having the amino acid sequence of SEQ ID NO: 75.

In some aspects, the second binding domain that specifically binds to TIGIT comprises a heavy chain having an amino acid sequence that is at least 90, 91, 92, 93, 94, 95, 96, 97, 98, or 99% identical to the amino acid sequence of SEQ ID NO: 73. In some aspects, the second binding domain that specifically binds to TIGIT comprises a light chain having an amino acid sequence that is at least 90, 91, 92, 93, 94, 95, 96, 97, 98, or 99% identical to the amino acid sequence of SEQ ID NO: 75.

In some aspects, the second binding domain that specifically binds TIGIT comprises a heavy chain variable domain that is encoded by the nucleic acid sequence of SEQ ID NO: 80 and a light chain variable domain that is encoded by the nucleic acid sequence of SEQ ID NO: 82.

In some aspects, the second binding domain that specifically binds TIGIT comprises a heavy chain variable domain that is encoded by a nucleic acid sequence that is at least 90, 91, 92, 93, 94, 95, 96, 97, 98, or 99% identical to SEQ ID NO: 80 and a light chain variable domain that is encoded by a nucleic acid sequence that is at least 90, 91, 92, 93, 94, 95, 96, 97, 98, or 99% identical to ID NO: 82.

In some aspects, the second binding domain that specifically binds to TIGIT comprises a heavy chain that is encoded by the nucleic acid sequence of SEQ ID NO: 81 and a light chain that is encoded by the nucleic acid sequence of SEQ ID NO: 83.

In some aspects, the second binding domain that specifically binds to TIGIT comprises a heavy chain that is encoded by a nucleic acid sequence that is at least 90, 91, 92, 93, 94, 95, 96, 97, 98, or 99% identical to SEQ ID NO: 26 and a light chain that is encoded by a nucleic acid sequence that is at least 90, 91, 92, 93, 94, 95, 96, 97, 98, or 99% identical to SEQ ID NO: 28.

### TIM-3 Binding Domains

The term T-cell immunoglobulin and mucin domain containing protein-3 (TIM-3) refers to a Type I cell-surface glycoprotein that comprises an N-terminal immunoglobulin (Ig)-like domain, a mucin domain with O-linked glycosylation and with N-linked glycosylation close to the membrane, a single transmembrane domain, and a cytoplasmic region with tyrosine phosphorylation motif(s). TIM-3 is a member of the T cell/transmembrane, immunoglobulin, and mucin (TIM) gene family. The amino acid sequence of the IgV domain of human TIM-3 is shown as SEQ ID NO: 102. The amino acid sequence of the human TIM-3 protein, including the signal peptides, is shown as SEQ ID NO: 103.

In some aspects, the present disclosure provides an anti-TIM-3/PD-1 bispecific binding protein named “AZD7789” that comprises the sequences of monoclonal antibody 013-1 (TIM-3) and LO115 (PD-1) as described in U.S. Pat. No. 10,457,732. The bispecific binding protein comprises a first heavy chain comprising the amino acid sequence of SEQ ID NO: 92, a first light chain comprising the amino acid sequence of SEQ ID NO: 95, and a second heavy chain comprising the amino acid sequence of SEQ ID NO: 63, and a second light chain comprising the amino acid sequence of SEQ ID NO: 65. The sequences of anti-TIM-3 monoclonal antibodies 013-1 and clone 62 are also disclosed in the same U.S. Pat. No. 10,457,732.

In some aspects, the second binding domain specifically binds to the C′C″ and DE loops of the immunoglobulin variable (IgV) domain of TIM-3 or binds to the PS binding cleft (FG and CC′ loops) of the IgV domain of TIM-3. In some aspects, the second binding domain that specifically binds to TIM-3 comprises Complementarity-Determining Regions (CDRs): HCDR1, HCDR2, HCDR3, LCDR1, LCDR2, and LCDR3 comprising the amino acid sequences of SEQ ID NOs: 84, 85, 86, 87, 88, and 89, respectively, or SEQ ID NOs: 84, 85, 86, 87, 88, and 90, respectively. In some aspects, the second binding domain that specifically binds to TIM-3 comprises HCDR1, HCDR2, HCDR3, LCDR1, LCDR2, and LCDR3 that are at least 90, 91, 92, 93, 94, 95, 96, 97, 98, or 99% identical to the amino acid sequences of SEQ ID NOs: 84, 85, 86, 87, 88, and 89, respectively, or SEQ ID NOs: 84, 85, 86, 87, 88, and 90, respectively. In some aspects, the second binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 102).

In some aspects, the second binding domain specifically binds to TIM-3 comprises a second heavy chain variable domain (VH) comprising the amino acid sequence of SEQ ID NO: 91, and a second light chain variable domain (VL) comprising the amino acid sequence of SEQ ID NO: 94. In some aspects, the second binding domain specifically binds to TIM-3 comprises a second heavy chain variable domain (VH) that are at least 90, 91, 92, 93, 94, 95, 96, 97, 98, or 99% identical to the amino acid sequence of SEQ ID NO: 91, and a second light chain variable domain (VL) that are at least 90, 91, 92, 93, 94, 95, 96, 97, 98, or 99% identical to the amino acid sequence of SEQ ID NO: 94. In some aspects, the second binding domain comprises a second heavy chain comprising the amino acid sequence of SEQ ID NO: 92, and a second light chain comprising the amino acid sequence of SEQ ID NO: 95. In some aspects, the second binding domain comprises a second heavy chain that are at least 90, 91, 92, 93, 94, 95, 96, 97, 98, or 99% identical to the amino acid sequence of SEQ ID NO: 92, and a second light chain that are at least 90, 91, 92, 93, 94, 95, 96, 97, 98, or 99% identical to the amino acid sequence of SEQ ID NO: 95.

In some aspects, the second binding domain comprises a first heavy chain comprising the amino acid sequence of SEQ ID NO: 96, and a first light chain comprising the amino acid sequence of SEQ ID NO: 97. In some aspects, the second binding domain comprises a first heavy chain that are at least 90, 91, 92, 93, 94, 95, 96, 97, 98, or 99% identical to the amino acid sequence of SEQ ID NO: 96, and a first light chain that are at least 90, 91, 92, 93, 94, 95, 96, 97, 98, or 99% identical to the amino acid sequence of SEQ ID NO: 97. In some aspects, the second binding domain comprises a first heavy chain comprising the amino acid sequence of SEQ ID NO: 98, a first light chain comprising the amino acid sequence of SEQ ID NO: 99. In some aspects, the second binding domain comprises a first heavy chain that are at least 90, 91, 92, 93, 94, 95, 96, 97, 98, or 99% identical to the amino acid sequence of SEQ ID NO: 98, a first light chain that are at least 90, 91, 92, 93, 94, 95, 96, 97, 98, or 99% identical to the amino acid sequence of SEQ ID NO: 99.

### CTLA-4 Binding Domains

As used herein, the terms “Cytotoxic T Lymphocyte associated Antigen-4,” “CTLA-4,” “CD152” and “hCTLA-4” are used interchangeably, and include variants, isoforms, species homologs of human CTLA-4. The complete CTLA-4 sequence can be found under NCBI Reference Sequence: NG_011502.1. The amino acid sequence of the human CTLA-4 protein is:

MACLGFQRHKAQLNLATRTWPCTLLFFLLFIPVFCKAMHVAQPAVVLASSR GIASFVCEYASPGKATEVRVTVLRQADSQVTEVCAATYMMGNELTFLDDSICTGTSSGN QVNLTIQGLRAMDTGLYICKVELMYPPPYYLGIGNGTQIYVIDPEPCPDSDFLLWILAAV SSGLFFYSFLLTAVSLSKMLKKRSPLTTGVYVKMPPTEPECEKQFQPYFIPIN (SEQ ID NO: 115). Cytotoxic T-lymphocyte-associated protein 4 (CTLA-4) is expressed on activated T cells and serves as a co-inhibitor to keep T-cell responses in check following CD28-mediated T cell activation. CTLA-4 is believed to regulate the amplitude of the early activation of naive and memory T cells following TCR engagement and to be part of a central inhibitory pathway that affects both antitumor immunity and autoimmunity. CTLA-4 is expressed exclusively on T cells, and the expression of its ligands CD80 (B7.1) and CD86 (B7.2), is largely restricted to antigen-presenting cells, T cells, and other immune mediating cells. Antagonistic anti-CTLA-4 antibodies that block the CTLA-4 signaling pathway have been reported to enhance T-cell activation.

In some aspects, the second binding domain specifically binds to CTLA-4. In some aspects, the bispecific binding protein is MEDI5752. The term MEDI5752 refers to an anti-PD-1/CTLA-4 bispecific antibody that comprises the light chain of SEQ ID NO:65 and the heavy chain of SEQ ID NO:63 (PD-1) and the light chain of SEQ ID NO:104 and the heavy chain of SEQ ID NO:105 (CTLA-4). MEDI5752 is disclosed in U.S. Pat. No. 10,457,732, which is herein incorporated by reference in its entirety.

In some aspects, the second binding domain comprises a VH CDR1 comprising the amino acid sequence of SEQ ID NO:109, a VH CDR2 comprising the amino acid sequence of SEQ ID NO:110, a VH CDR3 comprising the amino acid sequence of SEQ ID NO:111, a VL CDR1 comprising the amino acid sequence of SEQ ID NO:112, a VL CDR2 comprising the amino acid sequence of SEQ ID NO:113, and a VL CDR3 comprising the amino acid sequence of SEQ ID NO:114. In some aspects, the second binding domain comprises a VH CDR1 that are at least 90, 91, 92, 93, 94, 95, 96, 97, 98, or 99% identical to the amino acid sequence of SEQ ID NO:109, a VH CDR2 that are at least 90, 91, 92, 93, 94, 95, 96, 97, 98, or 99% identical to the amino acid sequence of SEQ ID NO:110, a VH CDR3 that are at least 90, 91, 92, 93, 94, 95, 96, 97, 98, or 99% identical to the amino acid sequence of SEQ ID NO:111, a VL CDR1 that are at least 90, 91, 92, 93, 94, 95, 96, 97, 98, or 99% identical to the amino acid sequence of SEQ ID NO:112, a VL CDR2 that are at least 90, 91, 92, 93, 94, 95, 96, 97, 98, or 99% identical to the amino acid sequence of SEQ ID NO:113, and a VL CDR3 that are at least 90, 91, 92, 93, 94, 95, 96, 97, 98, or 99% identical to the amino acid sequence of SEQ ID NO:114.

In some aspects, the second binding domain comprises a heavy chain comprising the amino acid sequence set forth in SEQ ID NO:105 and a light chain comprising the amino acid sequence set forth in SEQ ID NO:104.

In some aspects, the second binding domain comprises a heavy chain that are at least 90, 91, 92, 93, 94, 95, 96, 97, 98, or 99% identical to the amino acid sequence set forth in SEQ ID NO:105 and a light chain that are at least 90, 91, 92, 93, 94, 95, 96, 97, 98, or 99% identical to the amino acid sequence set forth in SEQ ID NO:104.

In some aspects, the bispecific binding protein comprises an IgG heavy chain constant region. In some aspects, the IgG heavy chain constant region is an IgG1 heavy chain constant region. In some aspects, the constant region includes mutations at L234F, L235E and P331S. In some aspects, the constant region comprises a knob mutation and a hole mutation, optionally wherein the knob mutation is in a heavy chain comprising a variable region that binds to CTLA-4 and the hole mutation is in a heavy chain comprising a variable region that binds to PD-1.

In some aspects, the bispecific binding protein is a humanized bispecific antibody or antigen-binding fragment thereof. In some aspects, the bispecific binding protein binds to human PD-1 and CTLA-4. In some aspects, the bispecific binding protein comprises the anti-PD-1 and anti-CTLA-4 heavy chain variable region (VH) CDR1, VH CDR2, VH CDR3, light chain variable region (VL) CDR1, VL CDR2, and VL CDR3 of sequences of MEDI5752.

### Aspects of Bispecific Proteins Binding PD-1

The bispecific binding protein having the PD-1 domain and one of the TIGIT, TIM-3 and CTLA-4 domains can be varied. In some aspects, the bispecific binding protein comprises an aglycosylated Fc region. In some aspects, the bispecific binding protein comprises a deglycosylated Fc region. In some aspects, the bispecific binding protein comprises an Fc region which has reduced fucosylation or is afucosylated. In some aspects, the bispecific binding protein is a full-length antibody. In some aspects, the antibody is an IgG antibody. In some aspects, the antibody is an IgG1 antibody. In some aspects, the antibody is human or humanized. In some aspects, the bispecific binding protein comprises a kappa light chain constant region. In some aspects, the bispecific binding protein comprises a lambda light chain constant region.

In some aspects, the disclosure provides a nucleic acid comprising a nucleotide sequence encoding a bispecific binding protein as described herein. In some aspects, the disclosure provides a host cell comprising a nucleic acid as described herein. In some aspects, the disclosure provides a host cell comprising a vector comprising a nucleic acid as described herein. In some aspects, the disclosure provides a host cell having a nucleic acid as described herein integrated into its genome. In some aspects, the disclosure provides a bispecific binding protein produced by a host cell as described herein.

### Types of Cancers

In some aspects of any of the methods disclosed herein, the methods are used to treat cancer. In some aspects, the cancer is one or more selected from ovarian cancer, breast cancer, uterine cancer, testicular cancer, bladder cancer, head and neck cancer, melanoma, renal cell carcinoma, pancreatic cancer, prostate cancer, cervical cancer, hematological cancer, endometrial cancer, cholangiocarcinoma, NSCLC (squamous and/or adenocarcinoma), gastrointestinal cancer such as gastric cancer and colorectal cancer, and lung cancer. In some aspects, the cancer is a breast cancer selected from hormone receptor-positive (HR+) breast cancer, human epidermal growth factor receptor 2 positive (HER2+) breast cancer, and triple negative breast cancer (TNBC). In some aspects, the cancer is homologous recombination deficient (HRD) cancer. In some aspects, the cancer comprises one or more cells having a mutation in an HRD gene selected from BRCA1, BRCA2, ATM, BRIP1, BARD1, CDK12, CHEK1, CHEK2, FANC1, PALB2, PPP2R2A, RAD51B, RAD51C, RAD51D, and RAD54L. In some aspects, the mutated HRD gene is selected from BRCA1, BRCA2, and ATM.

TABLE 1

| SEQ | | |
|---|---|---|
| ID NO. | Sequences | Description |
| 1 | GYYWN | ZY0EQD-E02; HCDR1 |
| 2 | EINHSGSTSYNPSLKS | ZY0EQD-E02; HCDR2 |
| 3 | VLYNWNVDS | ZY0EQD-E02; HCDR3 |
| 4 | RASQDIRNDVG | ZY0EQD-E02; LCDR1 |
| 5 | AASRLQS | ZY0EQD-E02; LCDR2 |
| 6 | LQHNSYPRT | ZY0EQD-E02; LCDR3 |
| 7 | GYYWN | ZY0EPQ-E02; HCDR1 |
| 8 | EINHSGSTNYNPSLKS | ZY0EPQ-E02; HCDR2 |
| 9 | NLYNWNLDS | ZY0EPQ-E02; HCDR3 |
| 10 | RASQGIRNDLG | ZY0EPQ-E02; LCDR1 |
| 11 | VASSLQS | ZY0EPQ-E02; LCDR2 |
| 12 | LQHNSYPRT | ZY0EPQ-E02; LCDR3 |
| 13 | SGGYYWS | ZY0EOB-F05; HCDR1 |
| 14 | NIYYSGSTYYNPSLKS | ZY0EOB-F05; HCDR2 |
| 15 | EKALATVTPSGYENYYTVDV | ZY0EOB-F05; HCDR3 |
| 16 | WASQGISSYLA | ZY0EOB-F05; LCDR1 |
| 17 | AASTLQS | ZY0EOB-F05; LCDR2 |
| 18 | QHLNSYPLT | ZY0EOB-F05; LCDR3 |
| 19 | SGGYYWS | ZY0EO5-E07; HCDR1 |
| 20 | NIYYSGSTYYNPSLKS | ZY0EO5-E07; HCDR2 |
| 21 | EKALASVIPSGYENYYVVDV | ZY0EO5-E07; HCDR3 |
| 22 | WASQGIAGYLA | ZY0EO5-E07; LCDR1 |
| 23 | AASTLQS | ZY0EO5-E07; LCDR2 |
| 24 | QHLNSYPLT | ZY0EO5-E07; LCDR3 |
| 25 | DYYMS | ZY0EP0-C07; HCDR1 |
| 26 | YISSSGSTIYYTDSVKG | ZY0EP0-C07; HCDR2 |
| 27 | DGVGFDY | ZY0EP0-C07; HCDR3 |
| 28 | RASQSVSSSYLA | ZY0EP0-C07; LCDR1 |
| 29 | AASSRAT | ZY0EP0-C07; LCDR2 |
| 30 | QQYGSSPLYT | ZY0EP0-C07; LCDR3 |

(ZY0EPQ-E02, variable heavy chain)
SEQ ID NO: 31
QVQLQQWGAGLLKPSETLSLTCAVYGGSFSGYYWNWIRQPPGKGLEWIGEINHSGSTNYNPSLK
SRVTILVDTSKNQFSLKLSSVTAADTAVYYCARNLYNWNLDSWGQGTLVTVSS
(ZY0EPQ-E02, variable light chain)
SEQ ID NO: 32
DIQMTQSPSSLSASVGDRVTITCRASQGIRNDLGWYQQKPGRAPKRLIYVASSLQSGVPSRFSGSG
SGTEFTLTISSLQPEDFATYYCLQHNSYPRTFGQGTKVEIK
(ZY0EQD-E02, variable heavy chain, e.g. pre-germlining)
SEQ ID NO: 33
QVQLQQWGAGLLKPSETLSLTCAVYGGSFSGYYWNWIRQPPGKGLEWIGEINHSGSTSYNPSLK
SRVTISVDTSKNQFSLKLSSVTAADTAVYYCARVLYNWNVDSWGQGTLVTVSS
(ZY0EQD-E02, variable light chain)
SEQ ID NO: 34
DIQMTQSPSSLSASVGDRVTITCRASQDIRNDVGWYQQKPGKAPKRLIYAASRLQSGVPSRFSGS
GSGTEFTLTISSLQPEDFATYYCLQHNSYPRTFGQGTKVEIK
(ZY0EOB-F05, variable heavy chain)
SEQ ID NO: 35
QVQLQESGPGLVKPSQTLSLTCTVSDGSISSGGYYWSWIRQHPGKGLEWIGNIYYSGSTYYNPSL
KSRVTISVDTSKNQFSLKLNSVTAADTAVYYCATEKALATVTPSGYENYYTVDVWGQGTTVTV
SS
(ZY0EOB-F05, variable light chain)
SEQ ID NO: 36
DIQLTQSPSFLSASVGDRVTITCWASQGISSYLAWYQQKPGKAPKLLIYAASTLQSGVPSRFSGSG
SGTEFTLTISSLQPEDFATYYCQHLNSYPLTFGGGTKVEIK
(ZY0EO5-E07, variable heavy chain)
SEQ ID NO: 37
QVQLQESGPGLVKPSQTLSLTCTVSGGSISSGGYYWSWIRQHPGKGLEWIGNIYYSGSTYYNPSL
KSRVTISVDTSKNQFSLKLSSVTAADTAVYYCAREKALASVIPSGYENYYVVDVWGQGTTVTVS
S
(ZY0EO5-E07, variable light chain)
SEQ ID NO: 38
DIQLTQSPSFLSASVGGRVTITCWASQGIAGYLAWYQQKPGKAPKLLIYAASTLQSGVPSRFSGS
GSGTEFTLTISSLQPEDFATYYCQHLNSYPLTFGGGTKVEIK
SEQ ID NO: 39: (ZY0EP0-C07, variable heavy chain)
QVQLVESGGVLVKPGGSLRLSCAASGFTLSDYYMSWIRQAPGMGLEWVSYISSSGSTIYYTDSV
KGRFTISRDSAKNSLYLQMNSLRAEDTAVYYCARDGVGFDYWGQGTLVTVSS
(ZY0EP0-C07, variable light chain)
SEQ ID NO: 40
EIVLTQSPGTLSLFPGERATLSCRASQSVSSSYLAWYQQKPGQSPRLLIYAASSRATGIPDRFSGSG
SGTDFTLTISRLEPEDFAVYYCQQYGSSPLYTFGQGTKLEIK
SEQ ID NO: 41 (Maia heavy chain constant region, cysteine insertion underlined):
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSCVFLFPPKP
KDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYT
QKSLSLSPGK
(light chain constant region)
SEQ ID NO: 42
RTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
(ZY0EQD-E02, variable heavy chain, e.g. pre-germlining, e.g. variant of SEQ ID NO: 33/
SEQ ID NO: 45)
SEQ ID NO: 43
QVQLQQWGAGLLKPSETLSLTCTVYGGSFSGYYWNWIRQPPGRGLEWIGEINHSGSTSYNPSLK
SRITISIDTSKNQFSLKLSSVTAADTAVYYCARVLYNWNVDSWGQGTLVTVSS
SEQ ID NO: 44 (ZY0EQD-E02, light chain):
DIQMTQSPSSLSASVGDRVTITCRASQDIRNDVGWYQQKPGKAPKRLIYAASRLQSGVPSRFSGS
GSGTEFTLTISSLQPEDFATYYCLQHNSYPRTFGQGTKVEIKRTVAAPSVFIFPPSDEQLKSGTASV
VCLLNNFYPREAKVQWKVDNALQSGNSQESVTEQDSKDSTYSLSSTLTLSKADYEKHKVYACE
VTHQGLSSPVTKSFNRGEC
(EQD-E02_GL, variable heavy chain, GL = germlined)
SEQ ID NO: 45
QVQLQQWGAGLLKPSETLSLACTVYGGSFSGYYWNWIRQPPGKGLEWIGEINHSGSTSYNPSLK
SRVTISVDTSKNQFSLKLSSVTAADTAVYYCARVLYNWNVDSWGQGTLVTVSS
(EQD-E02-GLY, variable heavy chain, GLY = germlined with a Y substitution)
SEQ ID NO: 46
QVQLQQWGAGLLKPSETLSLACTVYGGSFSGYYWNWIRQPPGKGLEWIGEIYHSGSTSYNPSLK
SRVTISVDTSKNQFSLKLSSVTAADTAVYYCARVLYNWNVDSWGQGTLVTVSS
(EQD-E02-GLQ, variable heavy chain, GLQ = germlined with a Q substitution)
SEQ ID NO: 47
QVQLQQWGAGLLKPSETLSLACTVYGGSFSGYYWNWIRQPPGKGLEWIGEIQHSGSTSYNPSLK
SRVTISVDTSKNQFSLKLSSVTAADTAVYYCARVLYNWNVDSWGQGTLVTVSS
(E02-GL-Maia-heavy chain, cysteine insertion underlined)
SEQ ID NO: 48
QVQLQQWGAGLLKPSETLSLACTVYGGSFSGYYWNWIRQPPGKGLEWIGEINHSGSTSYNPSLK
SRVTISVDTSKNQFSLKLSSVTAADTAVYYCARVLYNWNVDSWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSCVFLFPPKPKDTLMISRTPE
VTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGK
(E02-GLY-Maia-heavy chain, GLY = germlined with a Y substitution)
SEQ ID NO: 49
QVQLQQWGAGLLKPSETLSLACTVYGGSFSGYYWNWIRQPPGKGLEWIGEIYHSGSTSYNPSLK
SRVTISVDTSKNQFSLKLSSVTAADTAVYYCARVLYNWNVDSWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSCVFLFPPKPKDTLMISRTPE
VTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGK
(E02-GLQ-Maia-heavy chain, GLQ = germlined with a Q substitution)
SEQ ID NO: 50
QVQLQQWGAGLLKPSETLSLACTVYGGSFSGYYWNWIRQPPGKGLEWIGEIQHSGSTSYNPSLK
SRVTISVDTSKNQFSLKLSSVTAADTAVYYCARVLYNWNVDSWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSCVFLFPPKPKDTLMISRTPE
VTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGK
(E02-GL-WT-heavy chain)
SEQ ID NO: 51
QVQLQQWGAGLLKPSETLSLACTVYGGSFSGYYWNWIRQPPGKGLEWIGEINHSGSTSYNPSLK
SRVTISVDTSKNQFSLKLSSVTAADTAVYYCARVLYNWNVDSWGQGTLVTVSSASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSPGK
(heavy chain constant region)
SEQ ID NO: 52
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSCVFLFPPKP
KDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYT
QKSLSLSPGK
(Human B7H4 nucleic acid sequence, including 5' and 3' UTR)
SEQ ID NO: 53
GCCACCatggcttccctggggcagatcctcttctggagcataattagcatcatcattattctggctggagcaattgcactcatcattggcttt
ggtatttcagggagacactccatcacagtcactactgtcgcctcagctgggaacattggggaggatggaatcctgagctgcacttttgaacct
gacatcaaactttctgatatcgtgatacaatggctgaaggaaggtgttttaggcttggtccatgagttcaaagaaggcaaagatgagctgtcg
gagcaggatgaaatgttcagaggccggacagcagtgtttgctgatcaagtgatagttggcaatgcctctttgcggctgaaaaacgtgcaactc
acagatgctggcacctacaaatgttatatcatcacttctaaaggcaaggggaatgctaaccttgagtataaaactggagccttcagcatgccg
gaagtgaatgtggactataatgccagctcagagaccttgcggtgtgaggctccccgatggttcccccagcccacagtggtctgggcatcccaa
gttgaccagggagccaacttctcggaagtctccaataccagctttgagctgaactctgagaatgtgaccatgaaggttgtgtctgtgctctac
aatgttacgatcaacaacacatactcctgtatgattgaaaatgacattgccaaagcaacaggggatatcaaagtgacagaatcggagatcaaa
aggcggagtcacctacagctgctaaactcaaaggcttctctgtgtgtctcttctttctttgccatcagctgggcacttctgcctctcagccct
tacctgatgctaaaaTAATAA
(Human B7H4 nucleic acid sequence, coding sequence)
SEQ ID NO: 54
atggcttccctggggcagatcctcttctggagcataattagcatcatcattattctggctggagcaattgcactcatcattggctttggtatt
tcagggagacactccatcacagtcactactgtcgcctcagctgggaacattggggaggatggaatcctgagctgcacttttgaacctgacatc
aaactttctgatatcgtgatacaatggctgaaggaaggtgttttaggcttggtccatgagttcaaagaaggcaaagatgagctgtcggagcag
gatgaaatgttcagaggccggacagcagtgtttgctgatcaagtgatagttggcaatgcctctttgcggctgaaaaacgtgcaactcacagat
gctggcacctacaaatgttatatcatcacttctaaaggcaaggggaatgctaaccttgagtataaaactggagccttcagcatgccggaagtg
aatgtggactataatgccagctcagagaccttgcggtgtgaggctccccgatggttcccccagcccacagtggtctgggcatcccaagttgac
cagggagccaacttctcggaagtctccaataccagctttgagctgaactctgagaatgtgaccatgaaggttgtgtctgtgctctacaatgtt
acgatcaacaacacatactcctgtatgattgaaaatgacattgccaaagcaacaggggatatcaaagtgacagaatcggagatcaaaaggcgg
agtcacctacagctgctaaactcaaaggcttctctgtgtgtctcttctttctttgccatcagctgggcacttctgcctctcagcccttacctg
atgctaaaa
(Human B7H4 polypeptide sequence; UniProt Accession No .: Q7Z7D3)
SEQ ID NO: 55
MASLGQILFWSIISIIIILAGAIALIIGFGISGRHSITVTTVASAGNIGEDGILSCTFEPDIKLSDIVIQWL
KEGVLGLVHEFKEGKDELSEQDEMFRGRTAVFADQVIVGNASLRLKNVQLTDAGTYKCYIITSK
GKGNANLEYKTGAFSMPEVNVDYNASSETLRCEAPRWFPQPTVVWASQVDQGANFSEVSNTSF
ELNSENVTMKVVSVLYNVTINNTYSCMIENDIAKATGDIKVTESEIKRRSHLQLLNSKASLCVSSF
FAISWALLPLSPYLMLK

TABLE 2
Sequences of PD-1/TIGIT Bispecific

| SEQ | | |
|---|---|---|
| ID NO | | Sequence |
| 56 | PD1 LO115 | DYGMH |
| | HCDR1 | |
| 57 | PD1 LO115 | YISSGSYTIYSADSVKG |
| | HCDR2 | |
| 58 | PD1 LO115 | RAPNSFYEYYFDY |
| | HCDR3 | |
| 59 | PD1 LO115 | SASSKHTNLYWSRHMY |
| | LCDR1 | |
| 60 | PD1 LO115 | LTSNRAT |
| | LCDR2 | |
| 61 | PD1 LO115 | QQWSSNPFT |
| | LCDR3 | |
| 62 | PD1 LO115 | EVQLVESGGGLVQPGGSLRLSCAASGFTFSDYGMHWVRQAPGKG |
| | variable heavy | LEWVAYISSGSYTIYSADSVKGRFTISRDNAKNSLYLQMNSLRAED |
| | chain | TAVYYCARRAPNSFYEYYFDYWGQGTTVTVSS |
| 63 | PD1 LO115 | EVQLVESGGGLVQPGGSLRLSCAASGFTFSDYGMHWVRQAPGKG |
| | Hole heavy | LEWVAYISSGSYTIYSADSVKGRFTISRDNAKNSLYLQMNSLRAED |
| | chain | TAVYYCARRAPNSFYEYYFDYWGQGTTVTVSSASTKGPSVFPLAP |
| | | SSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCD |
| | | KTHTCPPCPAPEFEGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPASIEKTISKAKGQPREPQVCTLPPSREEM |
| | | TKNQVSLSCAVKGFYPSDIAVEWESNGQPENNYKTTPPVLDSDGS |
| | | FFLVSKLTVDKSRWQQGNVFSCSVMHEALHNHYTQKSLSLSPGK |
| 64 | PD1 LO115 | QIVLTQSPATLSLSPGERATLSCSASSKHTNLYWSRHMYWYQQKP |
| | variable light | GQAPRLLIYLTSNRATGIPARFSGSGSGTDFTLTISSLEPEDFAVYYC |
| | chain | QQWSSNPFTFGQGTKLEIK |
| 65 | PD1 LO115 | QIVLTQSPATLSLSPGERATLSCSASSKHTNLYWSRHMYWYQQKP |
| | WT Kappa light | GQAPRLLIYLTSNRATGIPARFSGSGSGTDFTLTISSLEPEDFAVYYC |
| | chain | QQWSSNPFTFGQGTKLEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| 66 | TIGIT VAR1 | SYAMH |
| | HCDR1 | |
| 67 | TIGIT VAR1 | VISYAGEVKYYADSVKG |
| | HCDR2 | |
| 68 | TIGIT VAR1 | DPLPLHYYGMDV |
| | HCDR3 | |
| 69 | TIGIT VAR1 | SGSSSNIGRRPVN |
| | LCDR1 | |
| 70 | TIGIT VAR1 | SQNQRPS |
| | LCDR2 | |
| 71 | TIGIT VAR1 | AVWDDIGRVLQ |
| | LCDR3 | |
| 72 | TIGIT VAR1 | QVQLVESGGGVVQPGRSLRLSCAASGFTFSSYAMHWVRQAPGKG |
| | variable heavy | LEWVAVISYAGEVKYYADSVKGRFTISRDNSKNTLYLQMNSLRAE |
| | chain | DTAVYYCARDPLPLHYYGMDVWGQGTTVTVSS |
| 73 | TIGIT VARI | QVQLVESGGGVVQPGRSLRLSCAASGFTFSSYAMHWVRQAPGKG |
| | Knob heavy | LEWVAVISYAGEVKYYADSVKGRFTISRDNSKNTLYLQMNSLRAE |
| | chain | DTAVYYCARDPLPLHYYGMDVWGQGTTVTVSSASTKGPSVCPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSV |
| | | DKTHTCPPCPAPEFEGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPASIEKTISKAKGQPREPQVYTLPPCREE |
| | | MTKNQVSLWCLVKGFYPSDIAVEWESNGQPENNYKTTPPVLDSD |
| | | GSFFLYSKLTVDKSRWQQGNVFSCSVMHEALHNHYTQKSLSLSPG |
| | | K |
| 74 | TIGIT VAR1 | QSVLTQPPSASGTPGQRVTISCSGSSSNIGRRPVNWYQQLPGTAPK |
| | variable light | LLIYSQNQRPSGVPDRFSGSQSGTSASLAISGLQSEDEADYFCAVW |
| | chain | DDIGRVLQLGGGTQLTVL |
| 75 | TIGIT VAR1 | QSVLTQPPSASGTPGQRVTISCSGSSSNIGRRPVNWYQQLPGTAPK |
| | engineered | LLIYSQNQRPSGVPDRFSGSQSGTSASLAISGLQSEDEADYFCAVW |
| | Lambda light | DDIGRVLQLGGGTQLTVLGQPKAAPSVTLFPPCSEELQANKATLV |
| | chain | CLISDFYPGAVTVAWKADSSPVKAGVETTTPSKQSNNKYAASSYL |
| | | SLTPEQWKSHRSYSCQVTHEGSTVEKTVAPTEVS |
| 76 | PD1 LO115 | GAGGTGCAGCTGGTGGAATCCGGCGGAGGACTGGTGCAGCCTG |
| | variable heavy | GCGGCTCCCTGAGACTGTCTTGCGCCGCCTCCGGCTTCACATTC |
| | chain | TCCGACTACGGCATGCACTGGGTCCGACAGGCCCCTGGAAAGG |
| | | GCCTGGAATGGGTGGCCTACATCTCCTCCGGCTCCTACACCATC |
| | | TACTCCGCCGACTCCGTGAAGGGCCGGTTCACCATCTCCCGGGA |
| | | CAACGCCAAGAACTCCCTGTACCTGCAGATGAACTCCCTGCGG |
| | | GCCGAGGACACAGCCGTGTACTACTGTGCCAGACGGGCCCCTA |
| | | ACTCCTTCTACGAGTACTACTTCGACTACTGGGGCCAGGGCACC |
| | | ACCGTGACCGTGTCCTCT |
| 77 | PD1 LO115 | GAGGTGCAGCTGGTGGAATCCGGCGGAGGACTGGTGCAGCCTG |
| | Hole heavy | GCGGCTCCCTGAGACTGTCTTGCGCCGCCTCCGGCTTCACATTC |
| | chain | TCCGACTACGGCATGCACTGGGTCCGACAGGCCCCTGGAAAGG |
| | | GCCTGGAATGGGTGGCCTACATCTCCTCCGGCTCCTACACCATC |
| | | TACTCCGCCGACTCCGTGAAGGGCCGGTTCACCATCTCCCGGGA |
| | | CAACGCCAAGAACTCCCTGTACCTGCAGATGAACTCCCTGCGG |
| | | GCCGAGGACACAGCCGTGTACTACTGTGCCAGACGGGCCCCTA |
| | | ACTCCTTCTACGAGTACTACTTCGACTACTGGGGCCAGGGCACC |
| | | ACCGTGACCGTGTCCTCTGCTAGCACCAAAGGTCCGAGCGTTTT |
| | | TCCGCTGGCACCGAGCAGCAAAAGCACCTCTGGTGGCACCGCA |
| | | GCACTGGGTTGTCTGGTGAAAGATTATTTTCCGGAACCGGTTAC |
| | | CGTTTCTTGGAATAGCGGTGCACTGACCAGCGGTGTTCATACCT |
| | | TTCCGGCAGTTCTGCAGAGCAGCGGTCTGTATAGCCTGTCTAGC |
| | | GTTGTTACCGTTCCGAGCAGCAGCCTGGGCACCCAGACCTATAT |
| | | TTGCAATGTGAATCATAAACCGAGCAATACAAAAGTTGATAAA |
| | | CGCGTTGAACCGAAAAGCTGTGACAAAACTCACACGTGCCCAC |
| | | CGTGCCCAGCACCTGAGTTCGAGGGGGGACCGTCAGTCTTCCTC |
| | | TTCCCCCCAAAACCCAAGGACACCCTCATGATCTCCCGGACCCC |
| | | TGAGGTCACATGCGTGGTGGTGGACGTGAGCCACGAAGACCCT |
| | | GAGGTCAAGTTCAACTGGTACGTGGACGGCGTGGAGGTGCATA |
| | | ATGCCAAGACAAAGCCGCGGGAGGAGCAGTACAACAGCACGT |
| | | ACCGTGTGGTCAGCGTCCTCACCGTCCTGCACCAGGACTGGCTG |
| | | AATGGCAAGGAGTACAAGTGCAAGGTCTCCAACAAAGCCCTCC |
| | | CAGCCAGCATCGAGAAAACCATCTCCAAAGCCAAAGGGCAGCC |
| | | CCGAGAACCACAGGTCTGCACCCTGCCCCCATCCCGGGAGGAG |
| | | ATGACCAAGAACCAGGTCAGCCTGAGCTGCGCGGTCAAAGGCT |
| | | TCTATCCCAGCGACATCGCCGTGGAGTGGGAGAGCAATGGGCA |
| | | GCCGGAGAACAACTACAAGACCACGCCTCCCGTGCTGGACTCC |
| | | GACGGCTCCTTCTTCCTCGTTAGCAAGCTCACCGTGGACAAGAG |
| | | CAGGTGGCAGCAGGGGAACGTCTTCTCATGCTCCGTGATGCAT |
| | | GAGGCTCTGCACAACCACTACACGCAGAAGAGCCTCTCCCTGT |
| | | CTCCGGGTAAATGA |
| 78 | PD1 LO115 | CAGATCGTGCTGACCCAGTCCCCTGCCACCCTGTCCCTGAGCCC |
| | variable light | TGGCGAGAGAGCCACCCTGAGCTGCTCCGCCTCCTCCAAGCAC |
| | chain | ACCAACCTGTACTGGTCCCGGCACATGTACTGGTATCAGCAGA |
| | | AGCCCGGCCAGGCCCCTCGGCTGCTGATCTACCTGACCTCTAAC |
| | | CGGGCCACCGGCATCCCTGCCAGATTCTCCGGCTCTGGCTCCGG |
| | | CACCGACTTCACCCTGACCATCTCCAGCCTGGAACCCGAGGACT |
| | | TCGCCGTGTACTACTGCCAGCAGTGGTCCTCCAACCCCTTCACC |
| | | TTCGGCCAGGGCACCAAGCTGGAAATCAAG |
| 79 | PD1 LO115 | CAGATCGTGCTGACCCAGTCCCCTGCCACCCTGTCCCTGAGCCC |
| | WT Kappa light | TGGCGAGAGAGCCACCCTGAGCTGCTCCGCCTCCTCCAAGCAC |
| | chain | ACCAACCTGTACTGGTCCCGGCACATGTACTGGTATCAGCAGA |
| | | AGCCCGGCCAGGCCCCTCGGCTGCTGATCTACCTGACCTCTAAC |
| | | CGGGCCACCGGCATCCCTGCCAGATTCTCCGGCTCTGGCTCCGG |
| | | CACCGACTTCACCCTGACCATCTCCAGCCTGGAACCCGAGGACT |
| | | TCGCCGTGTACTACTGCCAGCAGTGGTCCTCCAACCCCTTCACC |
| | | TTCGGCCAGGGCACCAAGCTGGAAATCAAGCGTACGGTGGCTG |
| | | CACCATCTGTCTTCATCTTCCCGCCATCTGATGAGCAGTTGAAA |
| | | TCTGGAACTGCCTCTGTTGTGTGCCTGCTGAATAACTTCTATCCC |
| | | AGAGAGGCCAAAGTACAGTGGAAGGTGGATAACGCCCTCCAAT |
| | | CGGGTAACTCCCAGGAGAGTGTCACAGAGCAGGACAGCAAGG |
| | | ACAGCACCTACAGCCTCAGCAGCACCCTGACGCTGAGCAAAGC |
| | | AGACTACGAGAAACACAAAGTCTACGCCTGCGAAGTCACCCAT |
| | | CAGGGCCTGAGCTCGCCCGTCACAAAGAGCTTCAACAGGGGAG |
| | | AGTGTTAG |
| 80 | TIGIT VAR1 | CAGGTGCAGCTGGTGGAGTCTGGAGGAGGCGTGGTCCAGCCTG |
| | variable heavy | GGAGGTCCCTGAGACTCTCCTGTGCAGCCTCTGGATTCACCTTC |
| | chain | AGTAGCTATGCTATGCACTGGGTCCGCCAGGCTCCAGGCAAGG |
| | | GGCTGGAGTGGGTGGCAGTTATATCATATGCGGGGGAGGTGAA |
| | | ATACTACGCAGACTCCGTGAAGGGCCGATTCACCATCTCCAGA |
| | | GACAATTCCAAGAACACGCTGTATCTGCAAATGAACAGCCTGA |
| | | GAGCTGAGGACACGGCTGTGTATTACTGTGCGAGAGACCCGCT |
| | | ACCGCTACATTACTACGGTATGGACGTCTGGGGCCAAGGGACC |
| | | ACGGTCACCGTCTCCTCA |
| 81 | TIGIT VAR1 | CAGGTGCAGCTGGTGGAGTCTGGAGGAGGCGTGGTCCAGCCTG |
| | Knob heavy | GGAGGTCCCTGAGACTCTCCTGTGCAGCCTCTGGATTCACCTTC |
| | chain | AGTAGCTATGCTATGCACTGGGTCCGCCAGGCTCCAGGCAAGG |
| | | GGCTGGAGTGGGTGGCAGTTATATCATATGCGGGGGAGGTGAA |
| | | ATACTACGCAGACTCCGTGAAGGGCCGATTCACCATCTCCAGA |
| | | GACAATTCCAAGAACACGCTGTATCTGCAAATGAACAGCCTGA |
| | | GAGCTGAGGACACGGCTGTGTATTACTGTGCGAGAGACCCGCT |
| | | ACCGCTACATTACTACGGTATGGACGTCTGGGGCCAAGGGACC |
| | | ACGGTCACCGTCTCCTCAGCGTCGACCAAAGGTCCGAGCGTGT |
| | | GCCCGCTGGCACCGAGCAGCAAAAGCACCTCTGGTGGCACCGC |
| | | AGCACTGGGTTGTCTGGTGAAAGATTATTTTCCGGAACCGGTTA |
| | | CCGTTTCTTGGAATAGCGGTGCACTGACCAGCGGTGTTCATACC |
| | | TTTCCGGCAGTCCTGCAGAGCAGCGGTCTGTATAGCCTGTCTAG |
| | | CGTTGTTACCGTTCCGAGCAGCAGCCTGGGCACCCAGACCTATA |
| | | TTTGCAATGTGAATCATAAACCGAGCAATACCAAAGTTGATAA |
| | | ACGCGTTGAACCGAAAAGCGTGGACAAAACTCACACGTGCCCA |
| | | CCGTGCCCAGCACCTGAGTTCGAGGGGGGACCGTCAGTCTTCCT |
| | | CTTCCCCCCAAAACCCAAGGACACCCTCATGATCTCCCGGACCC |
| | | CTGAGGTCACATGCGTGGTGGTGGACGTGAGCCACGAAGACCC |
| | | TGAGGTCAAGTTCAACTGGTACGTGGACGGCGTGGAGGTGCAT |
| | | AATGCCAAGACAAAGCCGCGGGAGGAGCAGTACAACAGCACG |
| | | TACCGTGTGGTCAGCGTCCTCACCGTCCTGCACCAGGACTGGCT |
| | | GAATGGCAAGGAGTACAAGTGCAAGGTCTCCAACAAAGCCCTC |
| | | CCAGCCAGCATCGAGAAAACCATCTCCAAAGCCAAAGGGCAGC |
| | | CCCGAGAACCACAGGTCTACACCCTGCCCCCATGCCGGGAGGA |
| | | GATGACCAAGAACCAGGTCAGCCTGTGGTGCCTGGTCAAAGGC |
| | | TTCTATCCCAGCGACATCGCCGTGGAGTGGGAGAGCAATGGGC |
| | | AGCCGGAGAACAACTACAAGACCACGCCTCCCGTGCTGGACTC |
| | | CGACGGCTCCTTCTTCCTCTATAGCAAGCTCACCGTGGACAAGA |
| | | GCAGGTGGCAGCAGGGGAACGTCTTCTCATGCTCCGTGATGCA |
| | | TGAGGCTCTGCACAACCACTACACGCAGAAGAGCTTAAGCCTG |
| | | TCTCCGGGTAAATGA |
| 82 | TIGIT VAR1 | CAGTCTGTGCTGACTCAGCCTCCCTCAGCGTCTGGGACCCCCGG |
| | variable light | GCAGAGGGTCACCATCTCTTGTTCTGGAAGCAGCTCCAATATCG |
| | chain | GAAGGAGGCCTGTAAACTGGTACCAGCAGCTCCCAGGAACGGC |
| | | CCCCAAACTCCTCATCTATAGTCAGAATCAGCGGCCCTCAGGGG |
| | | TCCCTGACCGATTCTCTGGCTCCCAGTCTGGCACCTCAGCCTCC |
| | | CTGGCCATCAGTGGGCTCCAGTCTGAGGATGAGGCTGATTATTT |
| | | CTGTGCAGTTTGGGATGACATCGGCCGCGTCCTGCAGTTAGGCG |
| | | GAGGCACCCAGCTGACCGTCCTA |
| 83 | TIGIT VAR1 | CAGTCTGTGCTGACTCAGCCTCCCTCAGCGTCTGGGACCCCCGG |
| | engineered | GCAGAGGGTCACCATCTCTTGTTCTGGAAGCAGCTCCAATATCG |
| | Lambda light | GAAGGAGGCCTGTAAACTGGTACCAGCAGCTCCCAGGAACGGC |
| | chain | CCCCAAACTCCTCATCTATAGTCAGAATCAGCGGCCCTCAGGGG |
| | | TCCCTGACCGATTCTCTGGCTCCCAGTCTGGCACCTCAGCCTCC |
| | | CTGGCCATCAGTGGGCTCCAGTCTGAGGATGAGGCTGATTATTT |
| | | CTGTGCAGTTTGGGATGACATCGGCCGCGTCCTGCAGTTAGGCG |
| | | GAGGCACCCAGCTGACCGTCCTAGGTCAGCCCAAGGCGGCCCC |
| | | CTCGGTCACTCTGTTCCCGCCCTGCTCTGAGGAGCTTCAAGCCA |
| | | ACAAGGCCACACTGGTGTGTCTCATAAGTGACTTCTACCCGGGA |
| | | GCCGTGACAGTGGCCTGGAAGGCAGATAGCAGCCCCGTCAAGG |
| | | CGGGAGTGGAGACCACCACACCCTCCAAACAAAGCAACAACAA |
| | | GTACGCGGCCAGCAGCTACCTGAGCCTGACGCCTGAGCAGTGG |
| | | AAGTCCCACAGAAGCTACAGCTGCCAGGTCACGCATGAAGGGA |
| | | GCACCGTGGAGAAGACAGTGGCCCCTACAGAAGTGTCATGA |

TABLE 3
PD-1/TIM-3 Bispecific

| SEQ ID | | |
|---|---|---|
| NO | Description | Amino Acid Sequence |
| 84 | AZD 7789 | SYAMS |
| | TIM-3 | |
| | VH CDR1 | |
| 85 | AZD 7789 | AISGSGGSTYYADSVKG |
| | TIM-3 | |
| | VH CDR2 | |
| 86 | AZD 7789 | GSYGTYYGNYFEY |
| | TIM-3 | |
| | VH CDR3 | |
| 56 | AZD 7789 | DYGMH |
| | PD-1 | |
| | VH CDR1 | |
| 57 | AZD 7789 | YISSGSYTIYSADSVKG |
| | PD-1 | |
| | VH CDR2 | |
| 58 | AZD 7789 | RAPNSFYEYYFDY |
| | PD-1 | |
| | VH CDR3 | |
| 87 | AZD 7789 | GGDNIGGKSVH |
| | TIM-3 | |
| | VL CDR1 | |
| 88 | AZD 7789 | YDSDRPS |
| | TIM-3 | |
| | VL CDR2 | |
| 89 | AZD 7789 | QVLDRRSDHFL |
| | TIM-3 | |
| | VL CDR3 | |
| 59 | AZD 7789 | SASSKHTNLYWSRHMYWY |
| | PD-1 | |
| | VL CDR1 | |
| 60 | AZD 7789 | LTSNRAT |
| | PD-1 | |
| | VL CDR2 | |
| 61 | AZD 7789 | QQWSSNP |
| | PD-1 | |
| | VL CDR3 | |
| 90 | TIM-3 (#62) | QVLDRRSDHWL |
| | VL CDR3 | |
| 91 | AZD 7789 | EVQLLESGGGLVQPGGSLRLSCAASGFTFSSYAMSWVRQ |
| | | APGKGLEWVSAISGSGGSTYYADSVKGRFTISRDNSKNTL |
| | TIM-3 | YLQMNSLRAEDTAVYYCARGSYGTYYGNYFEYWGQGTL |
| | VH | VTVSS |
| 92 | AZD 7789 | EVQLLESGGGLVQPGGSLRLSCAASGFTFSSYAMSWVRQ |
| | | APGKGLEWVSAISGSGGSTYYADSVKGRFTISRDNSKNTL |
| | TIM-3 | YLQMNSLRAEDTAVYYCARGSYGTYYGNYFEYWGQGTL |
| | | VTVSSASTKGPSVCPLAPSSKSTSGGTAALGCLVKDYFPEP |
| | HC | VTVSWNSGALTSGVHTFPAVLQSSGLYSLSSVVTVPSSSL |
| | | GTQTYICNVNHKPSNTKVDKRVEPKSVDKTHTCPPCPAPE |
| | | FEGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPASIEKTISKAKGQPREPQVYTLP |
| | | PCREEMTKNQVSLWCLVKGFYPSDIAVEWESNGQPENNY |
| | | KTTPPVLDSDGSFFLYSKLTVDKSRWQQGNVFSCSVMHE |
| | | ALHNHYTQKSLSLSPGK |
| 93 | TIM3 (#62) | QTVLTQPPSVSVAPGKTASISCGGDNIGGKSVHWYQQKPG |
| | Variable | QAPVLVIYYDSDRPSGIPQRFSGSNSGNTATLTIHRVEAGD |
| | Light VL | EADYYCQVLDRRSDHWLFGGGTKLTVL |
| 94 | AZD 7789 | SYVLTQPPSVSVAPGKTARITCGGDNIGGKSVHWYQQKP |
| | TIM-3 | GQAPVLVIYYDSDRPSGIPERFSGSNSGNTATLTISRVEAG |
| | VL | DEADYYCQVLDRRSDHFLFGGGTKLTVL |
| 95 | AZD 7789 | SYVLTQPPSVSVAPGKTARITCGGDNIGGKSVHWYQQKP |
| | TIM-3 | GQAPVLVIYYDSDRPSGIPERFSGSNSGNTATLTISRVEAG |
| | LC | DEADYYCQVLDRRSDHFLFGGGTKLTVLGQPKAAPSVTL |
| | | FPPCSEELQANKATLVCLISDFYPGAVTVAWKADSSPVKA |
| | | GVETTTPSKQSNNKYAASSYLSLTPEQWKSHRSYSCQVTH |
| | | EGSTVEKTVAPTEVS |
| 62 | AZD | EVQLVESGGGLVQPGGSLRLSCAASGFTFSDYGMHWVRQ |
| | 7789 | APGKGLEWVAYISSGSYTIYSADSVKGRFTISRDNAKNSL |
| | PD-1 | YLQMNSLRAEDTAVYYCARRAPNSFYEYYFDYWGQGTT |
| | VH | VTVSS |
| 63 | AZD 7789 | EVQLVESGGGLVQPGGSLRLSCAASGFTFSDYGMHWVRQ |
| | PD-1 | APGKGLEWVAYISSGSYTIYSADSVKGRFTISRDNAKNSL |
| | HC | YLQMNSLRAEDTAVYYCARRAPNSFYEYYFDYWGQGTT |
| | | VTVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKRVEPKSCDKTHTCPPCPAPE |
| | | FEGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPASIEKTISKAKGQPREPQVCTLP |
| | | PSREEMTKNQVSLSCAVKGFYPSDIAVEWESNGQPENNY |
| | | KTTPPVLDSDGSFFLVSKLTVDKSRWQQGNVFSCSVMHE |
| | | ALHNHYTQKSLSLSPGK |
| 64 | AZD 7789 | QIVLTQSPATLSLSPGERATLSCSASSKHTNLYWSRHMYW |
| | PD-1 | YQQKPGQAPRLLIYLTSNRATGIPARFSGSGSGTDFTLTISS |
| | VL | LEPEDFAVYYCQQWSSNPFTFGQGTKLEIK |
| 65 | AZD 7789 | QIVLTQSPATLSLSPGERATLSCSASSKHTNLYWSRHMYW |
| | PD-1 | YQQKPGQAPRLLIYLTSNRATGIPARFSGSGSGTDFTLTISS |
| | LC | LEPEDFAVYYCQQWSSNPFTFGQGTKLEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| 96 | TIM-3 Heavy | EVQLVESGGGLVQPGGSLRLSCAASGFTFSDYGMHWVRQ |
| | Chain | APGKGLEWVAYISSGSYTIYSADSVKGRFTISRDNAKNSL |
| | | YLQMNSLRAEDTAVYYCARRAPNSFYEYYFDYWGQGTT |
| | | VTVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKRVEPKSCDKTHTCPPCPAPE |
| | | FEGGPSVFLFPPKPKDTLMISRTPEVTCVWDVSHEDPEVKF |
| | | NWYVDGVEVHNAKTKPREEQYNSTYRWSVLTVLHQDW |
| | | LNGKEYKCKVSNKALPASIEKTISKAKGQPREPQVYTLPPS |
| | | REEMTKNQVSLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGKGGGGSGGGGSEVQLLESGGGLVQP |
| | | GGSLRLSCAASGFTFSSYAMSWVRQAPGKCLEWVSAISGS |
| | | GGSTYYADSVKGRFTISRDNSKNTLYLQMNSLRAEDTAV |
| | | YYCARGSYGTYYGNYFEYWGQGTLVTVSSGGGGSGGGG |
| | | SGGGGSGGGGSSYVLTQPPSVSVAPGKTARITCGGDNIGG |
| | | KSVHWYQQKPGQAPVLVIYYDSDRPSGIPERFSGSNSGNT |
| | | ATLTISRVEAGDEADYYCQVLDRRSDHFLFGCGTKLTVL |
| 97 | TIM-3 Light | QIVLTQSPATLSLSPGERATLSCSASSKHTNLYWSRHMYW |
| | Chain | YQQKPGQAPRLLIYLTSNRATGIPARFSGSGSGTDFTLTISS |
| | Variable Region | LEPEDFAVYYCQQWSSNPFTFGQGTKLEIK |
| 98 | TIM-3 Heavy | EVQLVESGGGLVQPGGSLRLSCAASGFTFSDYGMHWVRQ |
| | Chain | APGKGLEWVAYISSGSYTIYSADSVKGRFTISRDNAKNSL |
| | | YLQMNSLRAEDTAVYYCARRAPNSFYEYYFDYWGQGTT |
| | | VTVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKRVEPKSCDKTHTCPPCPAPE |
| | | FEGGPSVFLFPPKPKDTLMISRTPEVTCVWDVSHEDPEVKF |
| | | NWYVDGVEVHNAKTKPREEQYNSTYRWSVLTVLHQDW |
| | | LNGKEYKCKVSNKALPASIEKTISKAKGQPREPQVYTLPPS |
| | | REEMTKNQVSLTCLVKGFYPSDIAVEWESNGGGGSGGGG |
| | | SEVQLLESGGGLVQPGGSLRLSCAASGFTFSSYAMSWVRQ |
| | | APGKCLEWVSAISGSGGSTYYADSVKGRFTISRDNSKNTL |
| | | YLQMNSLRAEDTAVYYCARGSYGTYYGNYFEYWGQGTL |
| | | VTVSSGGG |
| | | GSGGGGSGGGGSGGGGSSYVLTQPPSVSVAPGKTARITCG |
| | | GDNIGGKSVHWYQQKPGQAPVLVIYYDSDRPSGIPERFSG |
| | | SNSGNTATLTISRVEAGDEADYYCQVLDRRSDHFLFGCGT |
| | | KLTVLGGGGSGGGGSGQPENNYKTTPPVLDSDGSFFLYSK |
| | | LTVDKSRWQQGNVFSCSVMHEALHNHYTQKSLSLSPGK |
| 99 | TIM-3 Light | QIVLTQSPATLSLSPGERATLSCSASSKHTNLYWSRHMYW |
| | Chain | YQQKPGQAPRLLIYLTSNRATGIPARFSGSGSGTDFTLTISS |
| | | LEPEDFAVYYCQQWSSNPFTFGQGTKLEIKRTVAAPSVFIF |
| | | PPSDEQLKSGTASWCLLNNFYPREAKVQWKVDNALQSGN |
| | | SQESVTEQDSKDSTYSLSSTLTLSKADYEKHKVYACEVTH |
| | | QGLSSPVTKSFNRGEC |
| 100 | TIM3 (#62) | EVQLLESGGGLVQPGGSLRLSCAASGFTFSSYAMSWVRQ |
| | Variable | APGKGLEWVSAISGSGGSTYYADSVKGRFTISRDNSKNTL |
| | Heavy VH | YLQMNSLRAEDTAVYYCARGSYGTYYGNYFEYWGRGTL |
| | | VTVSS |
| 101 | Amino acid | MQIPQAPWPVVWAVLQLGWRPGWFLDSPDRPWNPPTFSP |
| | sequence | ALLVVTEGDNATFTCSFSNTSESFVLNWYRMSPSNQTDKL |
| | of human PD-1 | AAFPEDRSQPGQDCRFRVTQLPNGRDFHMSVVRARRNDS |
| | protein | GTYLCGAISLAPKAQIKESLRAELRVTERRAEVPTAHPSPS |
| | | PRPAGQFQTLVVGVVGGLLGSLVLLVWVLAVICSRAARG |
| | | TIGARRTGQPLKEDPSAVPVFSVDYGELDFQWREKTPEPP |
| | | VPCVPEQTEYATIVFPSGMGTSSPARRGSADGPRSAQPLRP |
| | | EDGHCSWPL |
| 102 | Human TIM-3 IgV | SEVEYRAEVGQNAYLPCFYTPAAPGNLVPVCWGKGACPV |
| | domain | FECGNVVLRTDERDVNYWTSRYWLNGDFRKGDVSLTIEN |
| | | VTLADSGIYCCRIQIPGIMNDEKFNLKLVIK |
| 103 | Human TIM-3 | MFSHLPFDCVLLLLLLLLTRSSEVEYRAEVGQNAYLPCFY |
| | protein | TPAAPGNLVPVCWGKGACPVFECGNVVLRTDERDVNYW |
| | | TSRYWLNGDFRKGDVSLTIENVTLADSGIYCCRIQIPGIMN |
| | | DEKFNLKLVIKPAKVTPAPTRQRDFTAAFPRMLTTRGHGP |
| | | AETQTLGSLPDINLTQISTLANELRDSRLANDLRDSGATIRI |
| | | GIYIGAGICAGLALALIFGALIFKWYSHSKEKIQNLSLISLA |
| | | NLPPSGLANAVAEGIRSEENIYTIEENVYEVEEPNEYYCYV |
| | | SSRQQPSQPLGCRFAMP |

TABLE 4
Full-length light chain amino acid sequences of PD-1/CTLA4 Bispecific

| SEQ ID | | |
|---|---|---|
| NO: | Antibody | Amino Acid Sequence |
| 65 | MEDI | QIVLTQSPATLSLSPGERATLSCSASSKHTNLYWSRHMYWYQQKPGQ |
| | 5752 | APRLLIYLTSNRATGIPARFSGSGSGTDFTLTISSLEPEDFAVYYCQQWS |
| | PD-1 | SNPFTFGQGTKLEIKRTVAAPSVFIFPPSDEQLKSGTASVVCLLNNFYPR |
| | | EAKVQWKVDNALQSGNSQESVTEQDSKDSTYSLSSTLTLSKADYEKH |
| | | KVYACEVTHQGLSSPVTKSFNRGEC |
| 104 | MEDI | DIQMTQSPSSLSASVGDRVTITCRASQSINSYLDWYQQKPGKAPKLLIY |
| | 5752 | AASSLQSGVPSRFSGSGSGTDFTLTISSLQPEDFATYYCQQYYSTPFTFG |
| | CTLA-4 | PGTKVEIKGQPKAAPSVTLFPPCSEELQANKATLVCLISDFYPGAVTVA |
| | | WKADSSPVKAGVETTTPSKQSNNKYAASSYLSLTPEQWKSHRSYSCQ |
| | | VTHEGSTVEKTVAPTEVS |

TABLE 5
Full-length heavy chain amino acid sequences PD-1/CTLA4 Bispecific

| SEQ ID | | |
|---|---|---|
| NO: | Antibody | Amino Acid Sequence |
| 63 | MEDI | EVQLVESGGGLVQPGGSLRLSCAASGFTFSDYGMHWVRQAPGKGLE |
| | 5752 | WVAYISSGSYTIYSADSVKGRFTISRDNAKNSLYLQMNSLRAEDTAV |
| | PD-1 | YYCARRAPNSFYEYYFDYWGQGTTVTVSSASTKGPSVFPLAPSSKSTS |
| | | GGTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCPA |
| | | PEFEGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVS |
| | | NKALPASIEKTISKAKGQPREPQVCTLPPSREEMTKNQVSLSCAVKGF |
| | | YPSDIAVEWESNGQPENNYKTTPPVLDSDGSFFLVSKLTVDKSRWQQ |
| | | GNVFSCSVMHEALHNHYTQKSLSLSPGK |
| 105 | MEDI | QVQLVESGGGVVQPGRSLRLSCAASGFTFSSYGMHWVRQAPGKGLE |
| | 5752 | WVAVIWYDGSNKYYADSVKGRFTISRDNSKNTLYLQMNSLRAEDTA |
| | CTLA-4 | VYYCARDPRGATLYYYYYGMDVWGQGTTVTVSSASTKGPSVCPLAP |
| | | SSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSVDKTHT |
| | | CPPCPAPEFEGGPSVFLFPPKPKDTLMISRTPEVTCVVVDVSHEDPEVK |
| | | FNWYVDGVEVHNAKTKPREEQYNSTYRVVSVLTVLHQDWLNGKEY |
| | | KCKVSNKALPASIEKTISKAKGQPREPQVYTLPPCREEMTKNQVSLW |
| | | CLVKGFYPSDIAVEWESNGQPENNYKTTPPVLDSDGSFFLYSKLTVDK |
| | | SRWQQGNVFSCSVMHEALHNHYTQKSLSLSPGK |

TABLE 6
VH CDR Amino Acid Sequences of PD-1/CTLA4 Bispecific

| Anti- | VH CDR1 | VH CDR2 | VH CDR3 |
|---|---|---|---|
| body | (SEQ ID NO:) | (SEQ ID NO:) | (SEQ ID NO:) |
| MEDI | GFTFSDYGMH | YISSGSYTIYSADSVKG | RAPNSFYEYYFD |
| 5752 | (SEQ ID NO: 106) | (SEQ ID NO: 107) | (SEQ ID NO: 108) |
| PD-1 | | | |
| MEDI | GFTFSSYGMH | VIWYDGSNKYYADSVKG | DPRGATLYYYYYGMDV |
| 5752 | (SEQ ID NO: 109) | (SEQ ID NO: 110) | (SEQ ID NO: 111) |
| CTLA-4 | | | |

¹The VH CDRs in Table 1 are determined according to Kabat.

TABLE 7
VL CDR Amino Acid Sequences of PD-1/CTLA4 Bispecific

| Anti- | VL CDR1 | VL CDR2 | VL CDR3 |
|---|---|---|---|
| body | (SEQ ID NO:) | (SEQ ID NO:) | (SEQ ID NO:) |
| MEDI | SASSKHTNLYWSRHMYWY | TSNRAT | QQWSSNP |
| 5752 | (SEQ ID NO: 59) | (SEQ ID NO: 60) | (SEQ ID NO: 61) |
| PD-1 | | | |
| MEDI | RASQSINSYLD | AASSLQS | QQYYSTP |
| 5752 | (SEQ ID NO: 112) | (SEQ ID NO: 113) | (SEQ ID NO: 114) |
| CTLA-4 | | | |

²The VL CDRs in Table 2 are determined according to Kabat.

| | |
|---|---|
| human CTLA- | MACLGFQRHKAQLNLATRTWPCTLLFFLLFIPVFCKAMHVAQP |
| 4 protein | AVVLASSRGIASFVCEYASPGKATEVRVTVLRQADSQVTEVCA |
| | ATYMMGNELTFLDDSICTGTSSGNQVNLTIQGLRAMDTGLYICK |
| | VELMYPPPYYLGIGNGTQIYVIDPEPCPDSDFLLWILAAVSSGLF |
| | FYSFLLTAVSLSKMLKKRSPLTTGVYVKMPPTEPECEKQFQPYFI |
| | PIN (SEQ ID NO: 115) |

TABLE 8
Amino acid sequences of EGFR/cMET Bispecific

| SEQ ID | | |
|---|---|---|
| NO | Description | Amino Acid Sequence |
| 116 | INT-017 | DNDFS |
| | Anti-EGFR | |
| | VH CDR1 | |
| 117 | INT-017 | AIVAVFRTETYAQKFQD |
| | Anti-EGFR | |
| | VH CDR2 | |
| 118 | INT-017 | RLMSAISGPGAPLLM |
| | Anti-EGFR | |
| | VH CDR3 | |
| 119 | INT-017 | TGTSSDVGGYNYVS |
| | Anti-EGFR | |
| | VL CDR1 | |
| 120 | INT-017 | DVSKRPS |
| | Anti-EGFR | |
| | VL CDR2 | |
| 121 | INT-017 | SSYTSSDTLEI |
| | Anti-EGFR | |
| | VL CDR3 | |
| 122 | INT-017 | DYYIH |
| | Anti-cMET | |
| | VH CDR1 | |
| 123 | INT-017 | WMNPNSGNTGYAQKFQG |
| | Anti-cMET | |
| | VH CDR2 | |
| 124 | INT-017 | GQGYTHS |
| | Anti-cMET | |
| | VH CDR3 | |
| 125 | INT-017 | RASEGIYHWLA |
| | Anti-cMET | |
| | VL CDR1 | |
| 126 | INT-017 | KASSLAS |
| | Anti-cMET | |
| | VL CDR2 | |
| 127 | INT-017 | QQYSNYPPT |
| | Anti-cMET | |
| | VL CDR3 | |
| 128 | INT-017 | QVQLVQSGAEVKKPGSSVKVSCKASGGTFSDNDFSWVRQ |
| | Anti-EGFR | APGQGLEWMGAIVAVFRTETYAQKFQDRVKITADISTRTT |
| | VH | YMELSSLRSEDTAVYYCARRLMSAISGPGAPLLMWGQGT |
| | | LVTVSS |
| 129 | INT-017 | QVQLVQSGAEVKKPGSSVKVSCKASGGTFSDNDFSWVRQ |
| | Anti-EGFR | APGQGLEWMGAIVAVFRTETYAQKFQDRVKITADISTRTT |
| | HC | YMELSSLRSEDTAVYYCARRLMSAISGPGAPLLMWGQGT |
| | | LVTVSSASTKGPSVCPLAPSSKSTSGGTAALGCLVKDYFPE |
| | | PVTVSWNSGALTSGVHTFPAVLQSSGLYSLSSVVTVPSSSL |
| | | GTQTYICNVNHKPSNTKVDKRVEPKSVDKTHTCPPCPAPE |
| | | FEGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPASIEKTISKAKGQPREPQVYTLP |
| | | PCREEMTKNQVSLWCLVKGFYPSDIAVEWESNGQPENNY |
| | | KTTPPVLDSDGSFFLYSKLTVDKSRWQQGNVFSCSVMHE |
| | | ALHNHYTQKSLSLSPGK |
| 130 | INT-017 | QSALTQPRSVSGSPGQSVTISCTGTSSDVGGYNYVSWYQQ |
| | Anti-EGFR | HPGKAPKLMIYDVSKRPSGVPDRFSGSKSGNTASLTISGLQ |
| | VL | AEDEADYYCSSYTSSDTLEIFGGGTKLTVLGQPKAAPSVT |
| | | L |
| 131 | INT-017 | QSALTQPRSVSGSPGQSVTISCTGTSSDVGGYNYVSWYQQ |
| | Anti-EGFR | HPGKAPKLMIYDVSKRPSGVPDRFSGSKSGNTASLTISGLQ |
| | LC | AEDEADYYCSSYTSSDTLEIFGGGTKLTVLGQPKAAPSVT |
| | | LFPPCSEELQANKATLVCLISDFYPGAVTVAWKADSSPVK |
| | | AGVETTTPSKQSNNKYAASSYLSLTPEQWKSHRSYSCQVT |
| | | HEGSTVEKTVAPTEVS |
| 132 | INT-017 | QVQLVQSGAEVKKPGASVKVSCKASGYTFTDYYIHWVR |
| | Anti-cMET | QATGQGLEWMGWMNPNSGNTGYAQKFQGRVTMTRDTS |
| | VH | ISTAYMELSSLRSEDTAVYYCARGQGYTHSWGQGTMVTV |
| | | SS |
| 133 | INT-017 | QVQLVQSGAEVKKPGASVKVSCKASGYTFTDYYIHWVR |
| | Anti-cMET | QATGQGLEWMGWMNPNSGNTGYAQKFQGRVTMTRDTS |
| | HC | ISTAYMELSSLRSEDTAVYYCARGQGYTHSWGQGTMVTV |
| | | SSASTKGPSVFPLAPSSKSTSGGTAALGCLVKDYFPEPVTV |
| | | SWNSGALTSGVHTFPAVLQSSGLYSLSSVVTVPSSSLGTQT |
| | | YICNVNHKPSNTKVDKRVEPKSCDKTHTCPPCPAPEFEGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPASIEKTISKAKGQPREPQVCTLPPSRE |
| | | EMTKNQVSLSCAVKGFYPSDIAVEWESNGQPENNYKTTP |
| | | PVLDSDGSFFLVSKLTVDKSRWQQGNVFSCSVMHEALHN |
| | | HYTQKSLSLSPGK |
| 134 | INT-017 | DIQMTQSPSTLSASVGDRVTITCRASEGIYHWLAWYQQKP |
| | Anti-cMET | GKAPKLLIYKASSLASGVPSRFSGSGSGTEFTLTISSLQPDD |
| | VL | FATYYCQQYSNYPPTFGGGTKLEIK |
| 135 | INT-017 | DIQMTQSPSTLSASVGDRVTITCRASEGIYHWLAWYQQKP |
| | Anti-cMET | GKAPKLLIYKASSLASGVPSRFSGSGSGTEFTLTISSLQPDD |
| | LC | FATYYCQQYSNYPPTFGGGTKLEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |

### EXAMPLES

### Example 1: TOP1i-ADCs Induce Tumor Antigenicity and Ligand Expression in Multiple In Vitro Model Systems

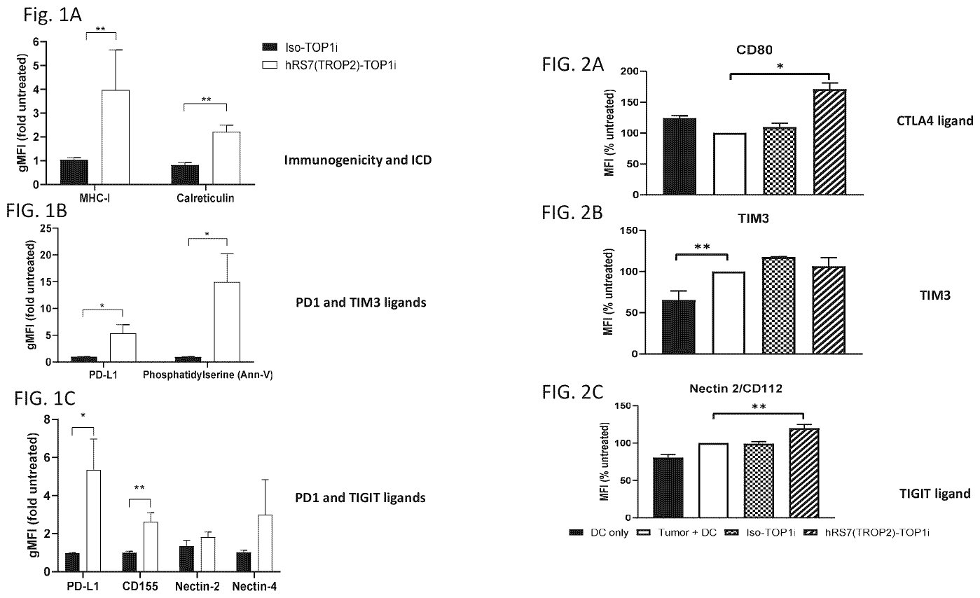

In vitro studies were performed to determine if antibody-drug conjugates (ADCs) increased tumor antigenicity and increased expression of certain immune-oncology (JO) ligands. MX1 tumor cells were cultured in vitro in DMIEM:F-12 media with 1000 Fetal Bovine Serum (FBS) at 37° C. and 500 CO₂ for 24 hours. Cultured cells were exposed to a control ADCs having a topoisomerase 1 inhibitor warhead (iso-TOP1i) along with ADCs having the same warheads targeting TROP2 (antibody clone hRS7, hRS7(TROP2)-TOP1i) at a concentration of 1 μg/ml for five days. Cells were harvested by trypsinization, stained with fluorescent labeled antibodies for various cell surface expressed ligands and evaluated by flow cytometry for each ligand measured. Statistical significance for the indicated comparisons is denoted as * p<0.05, ** p<0.01. Fold increase of the geometric mean fluorescence intensity of expression versus control is plotted in FIGS. 1A-1C. As shown in FIG. 1A, expression of the immunogenicity and immunogenic cell death markers MHC-I and calreticulin is significantly increased by the hRS7(TROP2)-TOP1i ADC (MHC-I, p=0.008; calreticulin, p=0.008). As shown in FIG. 1B, expression of PD-1 ligand PD-L1 and TIM3 ligand phosphatidylserine, as shown by binding of fluorescent labeled Annexin-V (Ann-V) protein, is significantly increased by both ADCs (PD-L1, p=0.029; calreticulin, p=0.0029). As shown in FIG. 1C, expression of the TIGIT ligands CD155, Nectin-2 and Nectin-4 is increased by both ADCs, with a significant increase observed for CD155 expression by hRS7(TROP2)-TOP1i (CD155, p=0.008).

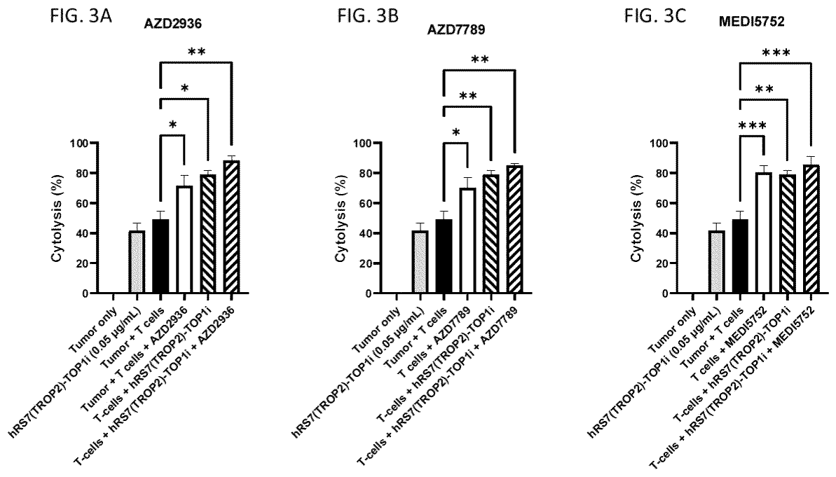

The same set of ADCs was tested for their ability to affect ligand expression on dendritic cells. Peripheral blood mononuclear cells (PBMC) were isolated from whole blood of HLA-A*02:01 positive healthy donors using a RoboSep™-S automated cell separator (StemCell Technologies, Cambridge, MA) in combination with an EasySep™ Direct Human PBMC isolation kit (StemCell Technologies), as per the manufacturer's instructions. Monocytes were subsequently isolated from PBMC using a negative selection magnetic microbead based assay kit (Miltenyi Biotec, Gaithersburg, MD). Immature monocyte derived dendritic cells were generated by culture of monocytes in RPMI-1640 media supplemented with 10% FBS, 50 ng/mL recombinant human interleukin 4 and 50 ng/mL recombinant human GM-CSF for 6 days with media replenish at day 3. Immature dendritic cells were then co-cultured with PC9 lung adenocarcinoma cells engineered to express the melanoma antigen MART-1 at a 1:1 ratio for 6 hours in RPMI-1640 media supplemented with 10% FBS. Cultured cells were exposed to the control and hRS7(TROP2)-TOP1i ADC at a concentration of 3 μg/mL for 72 hrs. Ligand expression was measured by flow cytometry as described in Example 1. Statistical significance for the indicated comparisons is denoted as * p<0.05, ** p<0.01. As shown in FIG. 2A, the hRS7(TROP2)-TOP1i ADC significantly increased expression of the CTLA4 ligand CD80 on dendritic cells (p=0.044). As shown in FIG. 2B, the co-culture of tumor cells and dendritic cells resulted in significantly increased TIM3 expression on dendritic cells (p=0.007). As shown in FIG. 2C, hRS7(TROP2)-TOP1i significantly increases expression of the TIGIT ligand Nectin2/CD112 on dendritic cells (p=0.006). These results demonstrate that the TOP1i ADCs are capable of modulating expression of antigens that can be targeted using immune-oncology therapies.

### Example 2: TOP1i ADC Combined with AZD2936 Enhances In Vitro T Cell Mediated Killing of Tumor Cells in Tumor-Immune Cell Co-Culture Model

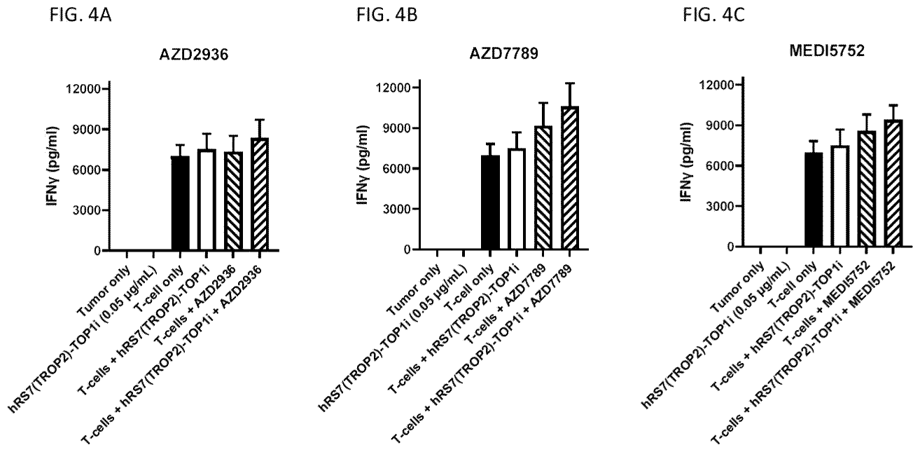

A co-culture assay system was developed which utilized melanoma antigen Melan A (MART-1) reactive T cells and PC9 lung adenocarcinoma cells engineered to express both the MART-1 antigen and Green Fluorescent Protein (GFP). Tumor cell lysis was evaluated utilizing the Sartorius Incucyte Live Cell Analysis System whereby loss of GFP signal intensity was used as a surrogate marker for cell death. Briefly, PC9-MART1-GFP cells were harvested from cell culture flasks using trypsin, washed once with complete RPMI media as defined in Example 1, resuspended and added to each well of 96 well clear bottom, opaque walled optical plates for 24 hours at 37° C. in 5% CO₂ as per the manufacturer's instructions. The PD-1-TIGIT (AZD2936), PD-1-TIM3 (AZD7789) or PD-1-CTLA4 (MEDI5752) bispecific antibodies were used at 2 nM concentration. The hRS7(TROP2)-TOP1i ADC was used at 0.05 μg/ml. Treatments were added alone or in combination to wells immediately before rested, antigen-specific CD8+ T cells were added at effector to target (E:T) ratios of 4:1 or 5:1 effector T cells to target tumor cells depending on the donor. Tumor cell death and percent cytolysis were measured five days after T cell addition by image analysis of GFP intensity per well. Data are from experiments with 3 unique donors. Statistical significance for the indicated comparisons is denoted as * p<0.05, ** p<0.01, p<0.001. As seen in FIG. 3A-C each bispecific antibody as well as the hRS7(TROP2)-TOP1i ADC enhanced T cell mediated cytolysis of PC9 tumor cells. In each case the highest degree of cytolysis was reached with the combination of bispecific antibody and the hRS7(TROP2)-TOP1i ADC.

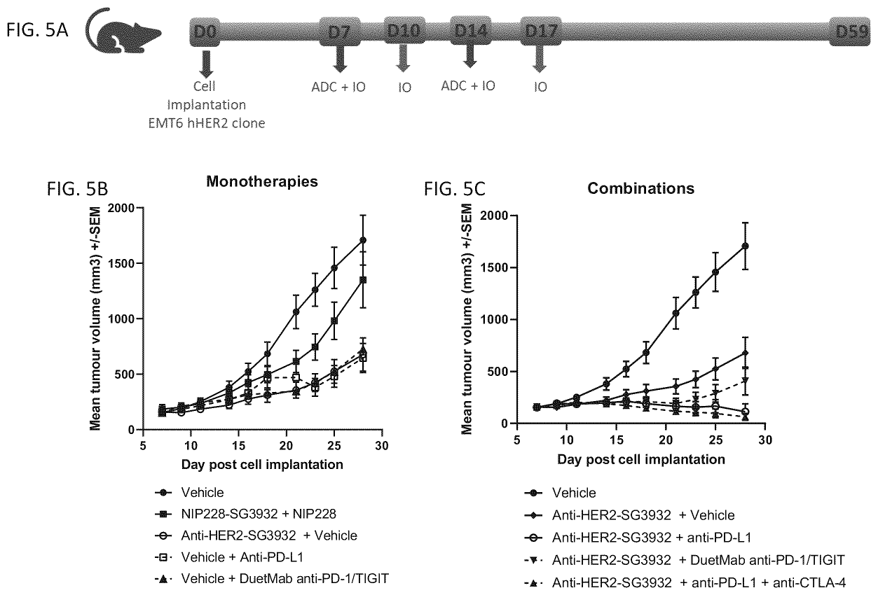

Antigen specific CD8+ T-cells secrete IFN-γ upon tumor antigen recognition. To evaluate the amount of IFN-7 detected within the supernatants of co-cultures containing PC9-MART1-GFP tumor cells and tumor reactive CD8+ T cells, separate parallel co-cultures were established in Nunc 96 well plates (Sigma Aldrich) alongside the Incucyte optical plates. For evaluation of IFN-7 release, supernatants were harvested from co-cultures in the Nunc 96 well plates 24, 72 and 144 hours later. IFN-7 was measured using Mesoscale Discovery multi-spot ELISA for human IFN-7 as per the manufacturer's instructions. As seen in FIG. 4A, T-cells potently induce IFN-7 secretion during co-culture of MART-1 antigen-specific T cells with antigen expressing tumor cells, whereas no IFN-7 secretion is measured in the absence of T-cells. For the MART-1 tumor antigen reactive T cell-PC9 lung adenocarcinoma co-culture, IFN-7 secretion by T cells was retained in the presence of the hRS7(TROP2)-TOP1i ADC alone, AZD2936 and MEDI5752 both alone and in combination with the ADC, FIG. 4C-4D. AZD7789 enhanced IFN-7, which was retained in combination with the hRS7(TROP2)-TOP1i ADC FIG. 4E.

### Example 3: In Vivo Combination Efficacy Observed with hHER2-TOP1i ADC and Anti-PD-L1 in EMT6-hHER2 Model

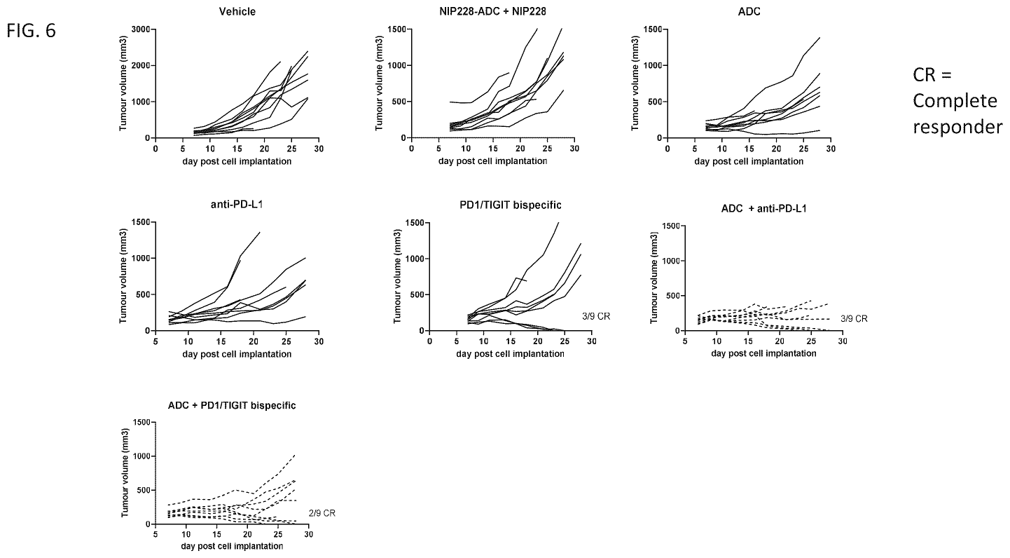

EMT6 murine breast cancer cells engineered to express human HER2 were implanted subcutaneously into the flank of female Balb/C mice on study day 0. Mice were kept in groups of no more than six animals per cage with continuous access to food and water. Nine mice were tested for each therapy. Monotherapy mice were administered the following: vehicle only, a NIP228 negative control antibody (10 mg/kg) and negative control ADC (NIP228-SG3932, 10 mg/kg), an anti-HER2 ADC (Anti-HER2-SG3932, 10 mg/kg), anti-PD-L1 antibody (10 mg/kg) and anti-PD-1/TIGIT bispecific antibody (10 mg/kg). With the exception of the vehicle only control, combination therapy mice were all administered the anti-HER2 ADC in combination with: vehicle, anti-PD-L1 antibody, anti-PD-1/TIGIT bispecific antibody (10 mg/kg each). Administration was done according to the schedule shown schematically in FIG. 5A, with ADC and IO therapies administered on days 7 and 14 and IO therapies only administered on days 10 and 17. Mean tumor volume was measured using digital calipers and averages were plotted in FIGS. 5B (monotherapies) and 5C (combinations). As seen by comparing FIGS. 5B and 5C, combination therapy greatly reduces mean tumor volume and growth. Mice were sacrificed on day 72.

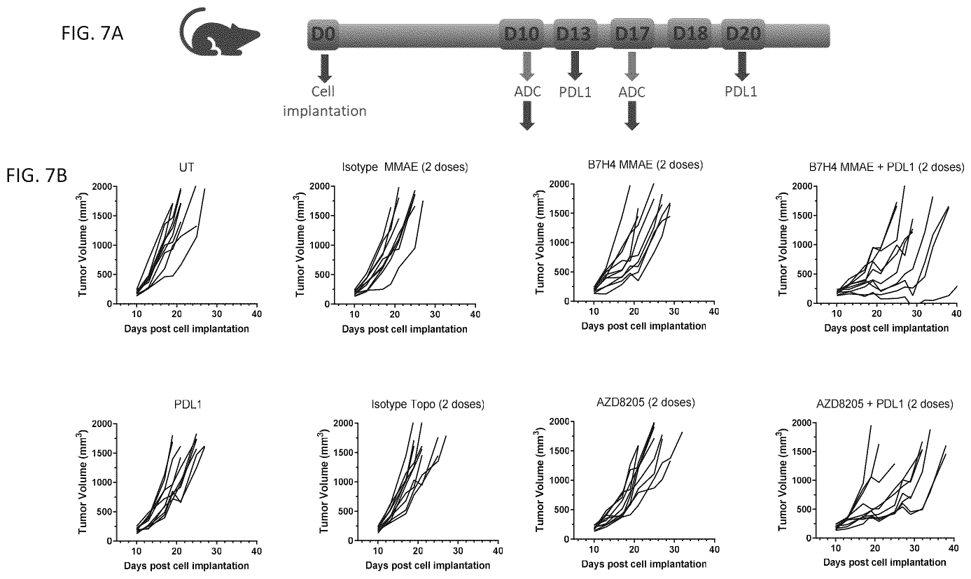

Plots of individual mouse tumor volumes for each therapy are shown in FIG. 6. As shown for some of the plots, there were complete responders (CR) to PD1/TIGIT bispecific monotherapy and ADC+PD-L1 antibody as well as ADC+PD1/TIGIT bispecific antibody combination therapies as indicated by the fraction next to the plots.

### Example 4: AZD8205 Combines with Anti-PD-L1 and Enhances Efficacy in Mouse B7114 Overexpressing Colon Carcinoma Mouse Model

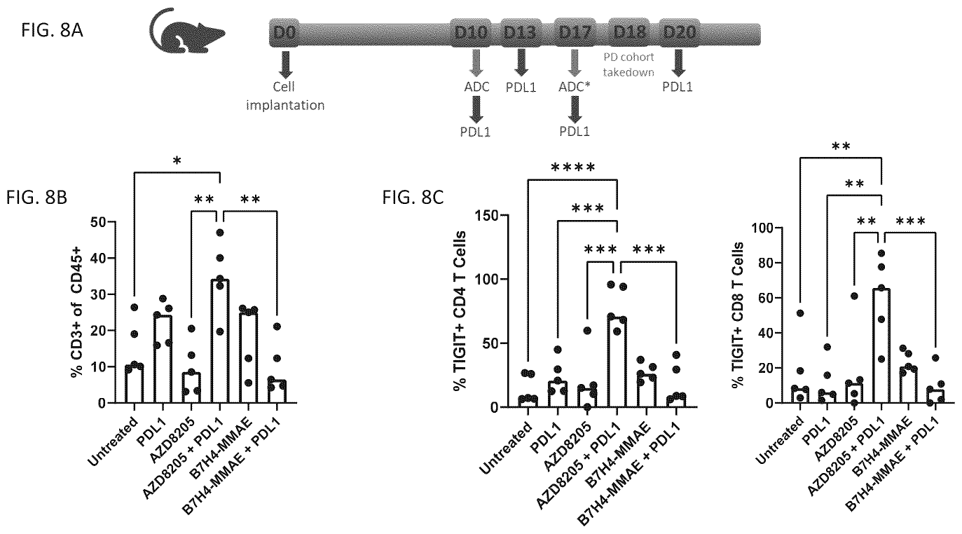

CT26 colon carcinoma cells overexpressing murine B7H4 were implanted subcutaneously into the flank of Balb/C mice on study day 0. Mice were housed and cared for as described in Example 3. Therapies were administered as shown schematically in FIG. 7A, with ADC administered on days 10 and 17 and anti-PD-L1 antibody administered on days 10, 13, 17 and 20. An ADC targeting B7H4 with a TOP1i warhead and linker (SG3932) is referred to as AZD8205. Mice were administered the following: untreated control (UT), anti-PD-L1 antibody alone (10 mg/kg), isotype monomethyl auristatin E (MMAE) warhead and TOP1i (SG3932) ADCs alone (7 mg/kg each), ADCs targeting B7H4 with either an MMAE or TOP1i (SG3932) warhead alone (7 mg/kg each), or combinations of anti-PD-L1 antibody with one of the ADCs (10 mg/kg anti-PD-L1, 7 mg/kg ADC). Mean tumor volume was measured and plotted in FIG. 7B. As seen in the figure, the combination treatment has enhanced efficacy against tumor cell growth in the model as compared to monotherapy treatments.

### Example 5: AZD8205+Anti-PD-L1 Combination Significantly Increases Tumor T Cells and TIGIT Expression in mB7-H4 CT26 Model

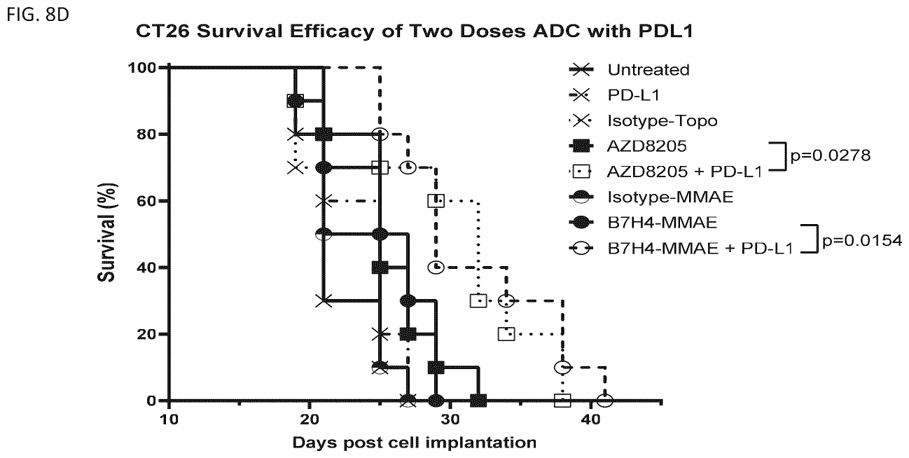

A separate cohort of mice from the study outlined in Example 4 was administered the same treatments as used in Example 4 with the administration schedule shown schematically in FIG. 8A. ADC and IO combination therapies were co-administered on days 10 and 17, while IO therapy only was administered on days 13 and 20. This cohort of mice was terminated at study day 18 for pharmacodynamic (PD) assessment of the immune cell composition of the tumor and tumor draining lymph nodes. Tumors and lymph nodes were dissociated to single cell suspensions and stained with fluorescently labeled antibodies specific for cell surface identity markers for mouse immune cell populations and analyzed by flow cytometry. The percentage of CD45+ immune cells was determined by dividing the number of CD45+ cells by the total number of live, single cells. Statistical significance for the indicated comparisons is denoted as * p<0.05, ** p<0.01, ***p<0.001 and ****p<0.0001. As shown in FIG. 8B, the combination of AZD8205 and anti-PD-L1 significantly increased the percentage of CD3+ T cells within the total CD45+ immune cell population (p=0.048 AZD8205+anti-PD-L1 vs untreated). The percentage of TIGIT+CD4 and TIGIT+CD8 T cells was determined by dividing the number of CD4 or CD8 and TIGIT double positive cells by the total number of CD4 or CD8 positive T cells. As shown in FIG. 8C, the combination of AZD8205 and anti-PD-L1 significantly increased the percentage of TIGIT+ T cells for both CD4+ and CD8+ T cells. Mouse survival rates for each group are plotted in FIG. 8D. There was a significant increase in median survival for the combination of AZD8205 with anti-PD-L1 compared to AZD8205 alone.

### Example 6: Robust Anti-Tumor Activity with Combination of B7-H4 ADC and PD-1-TIGIT Bispecific in mB7-H4 CT26 Model

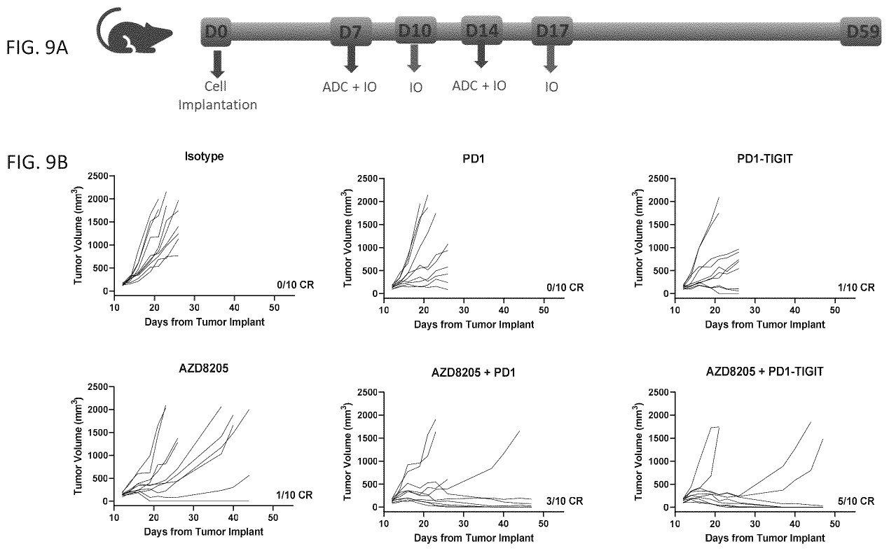

CT26 colon carcinoma cells overexpressing murine B7-H4 were implanted subcutaneously into the flank of Balb/C mice on study day 0 as described in Example 4. Therapies were administered as shown schematically in FIG. 9A, with ADC administered at 7 mg/kg on days 10 and 17 and anti-PD-1 or PD-1-TIGIT bispecific antibodies administered at 10 mg/kg on days 10, 13, 17 and 20. Plots of individual mouse tumor volumes for each therapy are shown in FIG. 9B. As shown for some of the plots, there were complete responders (CR) to PD-1-TIGIT bispecific monotherapy and AZD8205 monotherapy, however more complete responders to the combination AZD8205 was seen with the anti-PD1/TIGIT bispecific as indicated by the fraction next to the plot.

1. A method of treating cancer in a human subject in need thereof, comprising administering to the human subject:
a) an antibody-drug conjugate (ADC) comprising:
i) an antibody or antigen binding fragment thereof,
ii) a cleavable linker, and
iii) a cytotoxic agent;
wherein the cytotoxic agent is a compound of formula I: 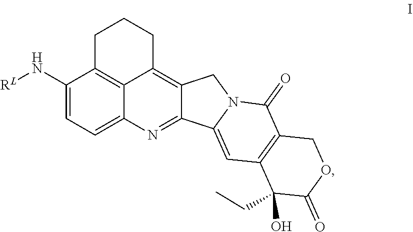
and salts and solvates thereof, wherein R^{L} is the cleavable linker, which is selected from: 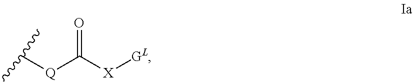
wherein Q is: 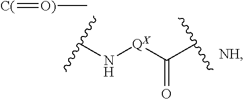
Where Q^{X} is such that Q is an amino-acid residue, a dipeptide residue, a tripeptide residue or a tetrapeptide residue;
wherein X is: 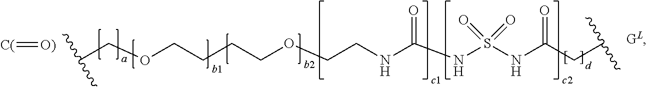
where a=0 to 5, b1=0 to 16, b2=0 to 16, c1=0 or 1, c2=0 or 1, d=0 to 5, at least b1 or b2=0, at least c1 or c2=0; and
wherein G^{L} is a linker for connecting to the antibody or antigen binding fragment; and 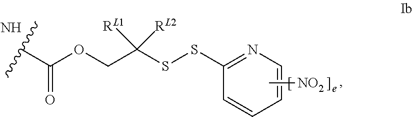
wherein R^{L1} and R^{L2} are independently selected from H and methyl, or together with the carbon atom to which they are bound form a cyclopropylene or cyclobutylene group, and e is 0 or 1; and
b) a bispecific checkpoint inhibitor.

2. A method of treating cancer in a human subject in need thereof, comprising administering to the human subject:
a) an antibody-drug conjugate (ADC) comprising:
i) an antibody or antigen binding fragment thereof which binds to a B7-H4 polypeptide,
ii) a cleavable linker, and
iii) a cytotoxic agent; and
b) a bispecific checkpoint inhibitor.

3. The method of claim 1 or claim 2, wherein the bispecific checkpoint inhibitor is a binding protein having a first binding domain that specifically binds to Programmed Death-1 (PD-1).

4. The method of any one of claims 1 to 3, wherein the antibody or antigen binding fragment thereof of the ADC comprises:
a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof, a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof,
a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof; or
a HCDR1, a HCDR2, a HCDR3, a LCDR1, a LCDR2, and a LCDR3 in a first binding arm comprising the amino acid sequence of SEQ ID NO: 116, SEQ ID NO: 117, SEQ ID NO: 118, SEQ ID NO: 119, SEQ ID NO: 120, and SEQ ID NO: 121; and a HCDR1, a HCDR2, a HCDR3, a LCDR1, a LCDR2, and a LCDR3 in a second binding arm comprising the amino acid sequence of SEQ ID NO: 122, SEQ ID NO: 123, SEQ ID NO: 124, SEQ ID NO: 125, SEQ ID NO: 126, and SEQ ID NO: 127.

5. The method of any one of claims 1 to 4, wherein the antibody or antigen binding fragment thereof of the ADC comprises:
a variable heavy (VH) chain and a variable light (VL) chain comprising the amino acid sequence of SEQ ID NO: 45 and SEQ ID NO: 34, respectively, or a functional variant thereof;
a variable heavy (VH) chain and a variable light (VL) chain comprising the amino acid sequence of SEQ ID NO: 33 and SEQ ID NO: 34, respectively, or a functional variant thereof;
a variable heavy (VH) chain and a variable light (VL) chain comprising the amino acid sequence of SEQ ID NO: 43 and SEQ ID NO: 34, respectively, or a functional variant thereof;
a variable heavy (VH) chain and a variable light (VL) chain comprising the amino acid sequence of SEQ ID NO: 46 and SEQ ID NO: 34, respectively, or a functional variant thereof;
a variable heavy (VH) chain and a variable light (VL) chain comprising the amino acid sequence of SEQ ID NO: 47 and SEQ ID NO: 34, respectively, or a functional variant thereof;
a VH chain and a VL chain comprising the amino acid sequence of SEQ ID NO: 31, and SEQ ID NO: 32, respectively, or a functional variant thereof,
a VH chain and a VL chain comprising the amino acid sequence of SEQ ID NO: 35 and SEQ ID NO: 36, respectively, or a functional variant thereof;
a VH chain and a VL chain comprising the amino acid sequence of SEQ ID NO: 37 and SEQ ID NO: 38, respectively, or a functional variant thereof; or
a VH chain and a VL chain comprising the amino acid sequence of SEQ ID NO: 39 and SEQ ID NO: 40, respectively, or a functional variant thereof, or
a VH chain and a VL chain in a first binding arm comprising the amino acid sequence of SEQ ID NO: 128 and SEQ ID NO: 130, respectively, or a functional variant thereof, and a VH chain and a VL chain in a second binding arm comprising the amino acid sequence of SEQ ID NO: 132 and SEQ ID NO: 134, respectively, or a functional variant thereof.

6. The method of any one of claims 1 to 5, wherein the antibody or antigen binding fragment thereof of the ADC comprises:
a VH chain and a VL chain comprising the amino acid sequence of SEQ ID NO: 45 and SEQ ID NO: 34, respectively, or a functional variant thereof, or
a VH chain and a VL chain in a first binding arm comprising the amino acid sequence of SEQ ID NO: 128 and SEQ ID NO: 130, respectively, or a functional variant thereof, and a VH chain and a VL chain in a second binding arm comprising the amino acid sequence of SEQ ID NO: 132 and SEQ ID NO: 134, respectively, or a functional variant thereof.

7. The method of any one of claims 1 to 6, wherein the antibody or antigen binding fragment thereof of the ADC binds an OVCAR4 cell line.

8. The method of any one of claims 1 to 7, wherein the antibody or antigen binding fragment thereof of the ADC comprises a heavy chain constant region comprising the amino acid sequence of SEQ ID NO: 41.

9. The method of any one of claims 1 to 7, wherein the antibody or antigen binding fragment thereof of the ADC comprises a heavy chain constant region comprising the amino acid sequence of SEQ ID NO: 52.

10. The method of any one of claims 1 to 7, wherein the antibody or antigen binding fragment thereof of the ADC comprises a light chain constant region comprising the amino acid sequence of SEQ ID NO: 42.

11. The method of any one of claims 1 to 7, wherein the antibody or antigen binding fragment thereof of the ADC comprises a heavy chain comprising the amino acid sequence of SEQ ID NO: 51; and a light chain comprising the amino acid sequence of SEQ ID NO: 44, or wherein the antibody or antigen binding fragment thereof of the ADC is a bispecific antibody or antigen binding fragment thereof and comprises a heavy chain and a light chain in a first binding arm comprising the amino acid sequence of SEQ ID NO: 129 and SEQ ID NO: 131, respectively, or a functional variant thereof, and a heavy chain and a light chain in a second binding arm comprising the amino acid sequence of SEQ ID NO: 133 and SEQ ID NO: 135, respectively, or a functional variant thereof.

12. The method of any one of claims 1 to 7, wherein the antibody or antigen binding fragment thereof of the ADC comprises a heavy chain comprising the amino acid sequence of SEQ ID NO: 48; and a light chain comprising the amino acid sequence of SEQ ID NO: 44.

13. The method of any one of claims 1 to 12, wherein the antibody or antigen binding fragment thereof of the ADC is a monoclonal antibody.

14. The method of any one of claims 1 to 13, wherein the antibody or antigen binding fragment thereof of the ADC is a humanized monoclonal antibody.

15. The method of any one of claims 1 to 14, wherein the cleavable linker of the ADC is an mp-PEG8-val-ala linker.

16. The method of any one of claims 2 to 15, wherein the cytotoxic agent of the ADC is topoisomerase inhibitor, tubulysin derivative, a pyrrolobenzodiazepine, or a combination thereof.

17. The method of any one of claims 2 to 16, wherein the cytotoxic agent of the ADC is a topoisomerase inhibitor.

18. The method of any one of claims 2 to 17, wherein the cytotoxic agent is a topoisomerase inhibitor and wherein the ii) cleavable linker and iii) cytotoxic agent of the ADC comprise a compound of formula I: 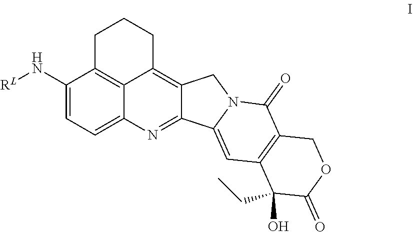
and salts and solvates thereof, wherein R^{L} is the cleavable linker, which is selected from: 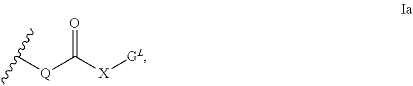
wherein Q is: 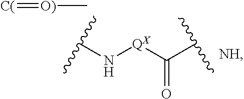
where Q^{X} is such that Q is an amino-acid residue, a dipeptide residue, a tripeptide residue or a tetrapeptide residue;
wherein X is: 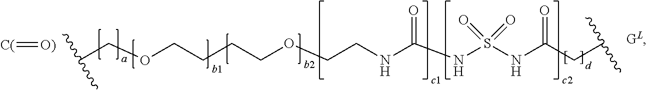
where a=0 to 5, b1=0 to 16, b2=0 to 16, c1=0 or 1, c2=0 or 1, d=0 to 5, at least b1 or b2=0, at least c1 or c2=0; and
wherein G^{L} is a linker for connecting to the antibody or antigen binding fragment; and 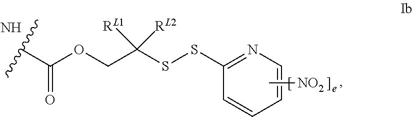
wherein R^{L1} and R^{L2} are independently selected from H and methyl, or together with the carbon atom to which they are bound form a cyclopropylene or cyclobutylene group, and e is 0 or 1.

19. The method of claim 1 or 18, wherein R^{L} is of formula Ia.

20. The method of claim 19, wherein Q is:
(a) an amino acid residue selected from: Phe, Lys, Val, Ala, Cit, Leu, Ile, Arg, and Trp; or
(b) a dipeptide residue selected from:
^{NH}-Phe-Lys-^{C═O},
^{NH}-Val-Ala-^{C═O},
^{NH}-Val-Lys-^{C═O},
^{NH} Ala-Lys-^{C═O},
^{NH}-Val-Cit-^{C═O},
^{NH}-Phe-Cit-^{C═O},
^{NH}-Leu-Cit-^{C═O},
^{NH}-Ile-Cit-^{C═O},
^{NH}-Phe-Arg-^{C═O},
^{NH}-Trp-Cit-^{C═O}, and
^{NH}-Gly-Val-^{C═O}; or
(c) a tripeptide residue selected from:
^{NH}-Glu-Val-Ala-^{C═O},
^{NH}-Glu-Val-Cit-^{C═O},
^{NH}-αGlu-Val-Ala-^{C═O}, and
^{NH}-αGlu-Val-Cit-^{C═O}; or
(d) a tetrapeptide residue selected from:
^{NH}-Gly-Gly-Phe-Gly ^{C═O}; and
^{NH}-Gly-Phe-Gly-Gly ^{C═O}.

21. The method of claim 19 or 20, wherein a is:
(a) 0 to 3; or
(b) 0 or 1; or
(c) 0.

22. The method of any one of claims 19 to 21, wherein b1 is:
(a) 0 to 8; or
(b) 0; or
(c) 2; or
(d) 3; or
(e) 4; or
(f) 5; or
(g) 8.

23. The method of any one of claims 19 to 22, wherein b2 is:
(a) 0 to 8; or
(b) 0; or
(c) 2; or
(d) 3; or
(e) 4; or
(f) 5; or
(g) 8.

24. The method of any to one of claims 1 and 18 to 23, wherein:
(a) c1 is 0 or 1;
(b) c2 is 0 or 1; and
(c) at least one of c1 and c2 is 0.

25. The method of any one of claims 1 and 18 to 24, wherein d is:
(a) 0 to 3; or
(b) 1 or 2; or
(c) 2; or
(d) 5.

26. The method of any one of claims 1 and 18 to 25, wherein:
(a) a is 0, b1 is 0, c1 is 1, c2 is 0 and d is 2, and b2 is 0, 2, 3, 4, 5 or 8; or
(b) a is 1, b2 is 0, c1 is 0, c2 is 0 and d is 0, and b1 is 0, 2, 3, 4, 5 or 8; or
(c) a is 0, b1 is 0, c1 is 0, c2 is 0 and d is 1, and b2 is 0, 2, 3, 4, 5 or 8; or
(d) b1 is 0, b2 is 0, c1 is 0, c2 is 0, one of a and d is 0, and the other of a and d is 1 or 5; or
(e) a is 1, b2 is 0, c1 is 0, c2 is 1, d is 2, and b1 is 0, 2, 3, 4, 5 or 8.

27. The method of any one of claims 1 and 18 to 26, wherein G^{L} is selected from: 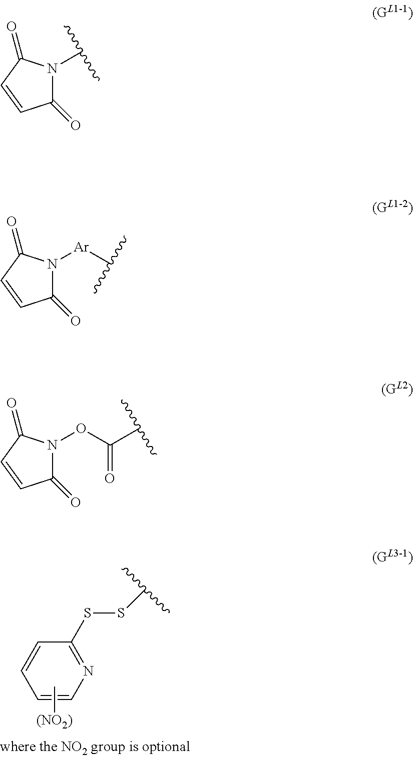 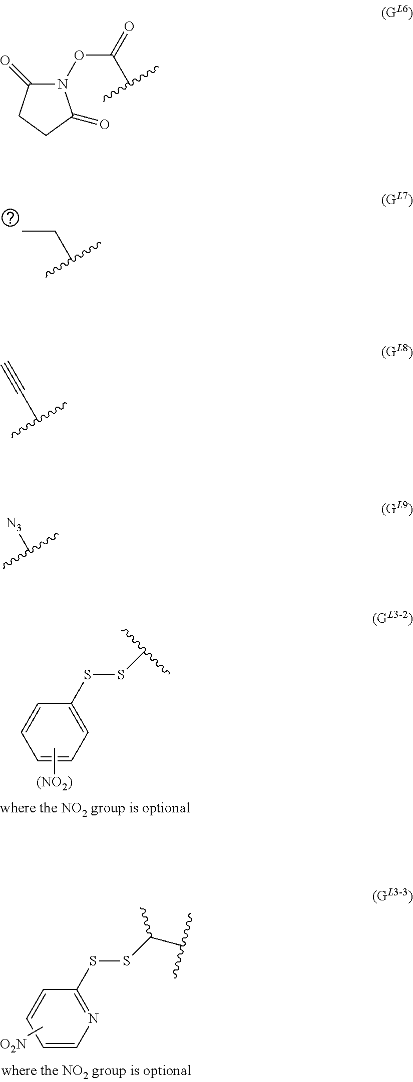 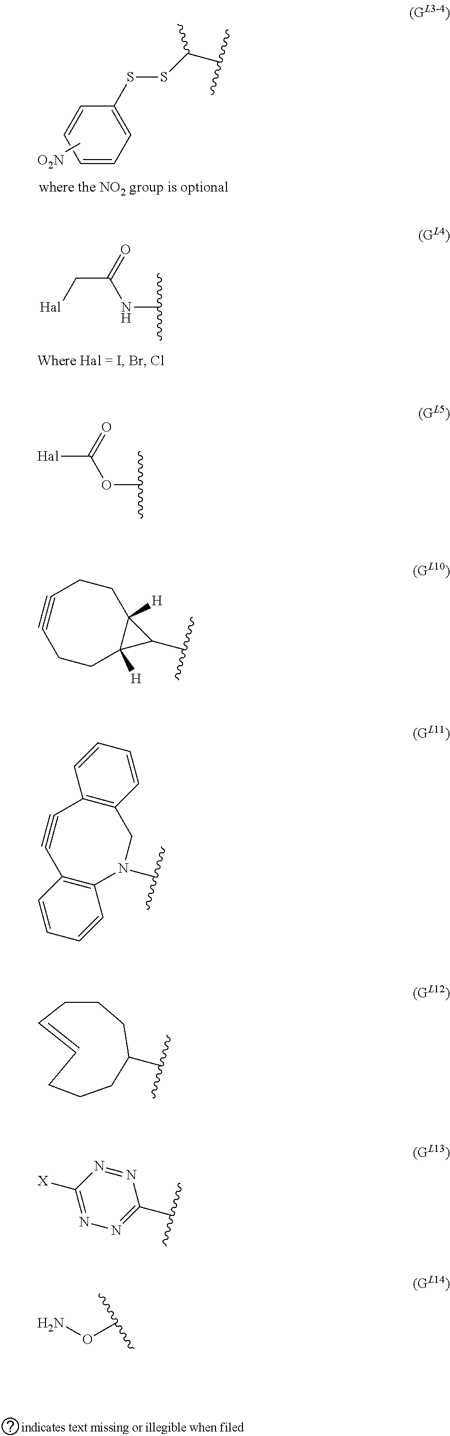
where Ar represents a C₅₋₆ arylene group, and X represents C₁₋₄ alkyl.

28. The method of claim 27, wherein G^{L} is selected from G^{L1-1} and G^{L1-2}.

29. The method of claim 1 or 18, wherein R^{L} is of formula Ib, and:
(a) both R^{L1} and R^{L2} are H; or
(b) R^{L1} is H and R^{L2} is methyl; or
(c) both R^{L1} and R^{L2} are methyl; or
(d) wherein R^{L1} and R^{L2} together with the carbon atom to which they are bound form a cyclopropylene group; or
(e) wherein R^{L1} and R^{L2} together with the carbon atom to which they are bound form a cyclobutylene group.

30. The method of claim 2, wherein the ADC is of formula IV:
L-(D^{L})ₚ (IV)
or a pharmaceutically acceptable salt or solvate thereof,
wherein L is the i) antibody or antigen binding fragment thereof, D^{L} is a drug linker unit comprising the ii) cleavable linker and the iii) cytotoxic agent, and D^{L} is of formula III: 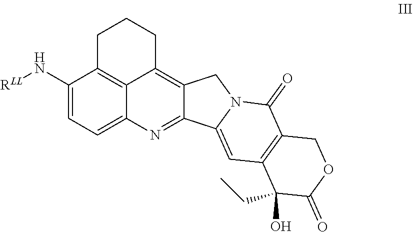
wherein R^{LL} is the cleavable linker selected from:
(ia′): 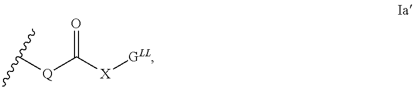
where Q and X are as defined in claim 1 or claim 29 and G^{LL} is a linker connected to the antibody or antigen binding fragment; and
(ib′): 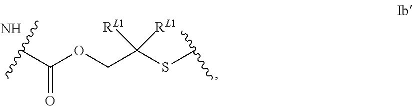
where R^{L1} and R^{L2} are as defined in either claim 1 or claim 29; and
p is an integer of from 1 to 20.

31. The method of claim 30, wherein G^{LL} is selected from: 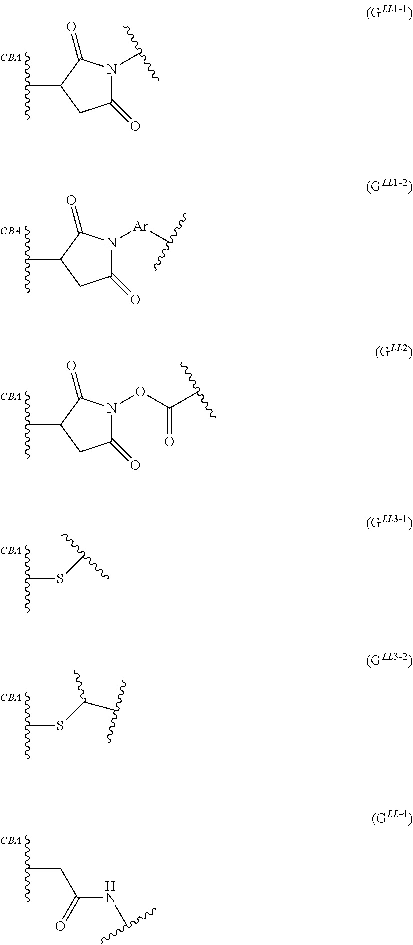 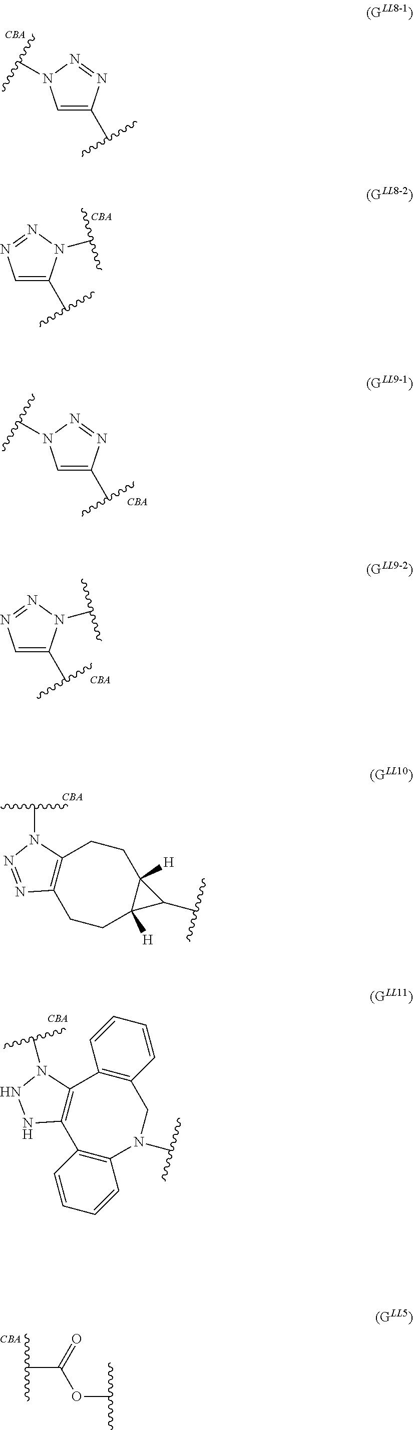 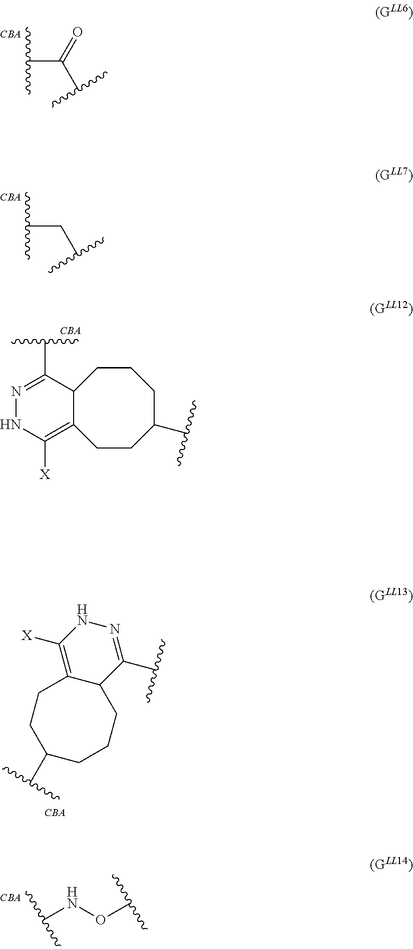
where Ar represents a C₅₋₆ arylene group and X represents C₁₋₄ alkyl.

32. The method of claim 31, wherein G^{LL} is selected from G^{LL1-1} and G^{LL1-2}.

33. The method of any one of claims 30 to 32, wherein the drug loading (p) of the cytotoxic agent to the antibody or antibody binding fragment is an integer from 1 to about 10.

34. The method of any one of claims 17 to 33, wherein the topoisomerase inhibitor is a compound of formula A: 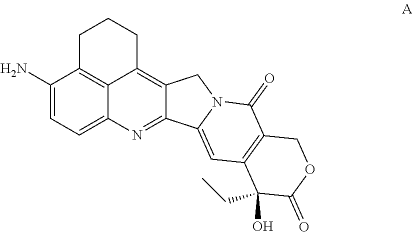 [SMILES: CC[C@@]1(O)C(=O)OCc2c1cc1n(c2=O)Cc2c-1nc1ccc(N)c3c1c2CCC3]
as a single enantiomer or in an enantiomerically enriched form.

35. The method of any one of claims 18 to 33, wherein the topoisomerase inhibitor is a compound with the formula VI: 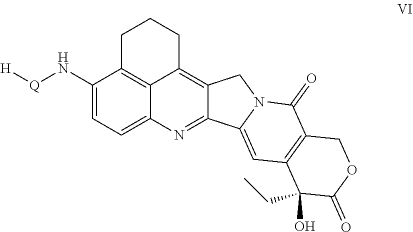
where Q is as in either claim 1 or claims 19-20.

36. The method of any one of claims 1 to 35, wherein the ii) cleavable linker and iii) cytotoxic agent together comprise the following compound:

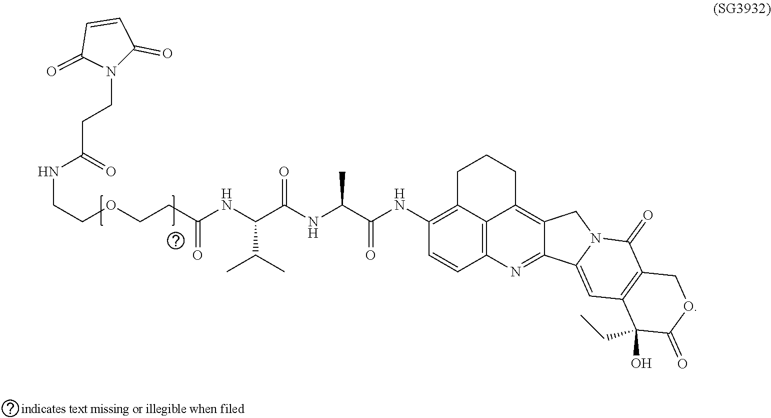

37. The method of any one of claims 1 to 36, wherein the bispecific checkpoint inhibitor comprises:
a) a first binding domain that specifically binds to PD-1 or PD-L1; and
b) a second binding domain that specifically binds to T cell immunoreceptor with Ig and ITIM domains (TIGIT), T-cell immunoglobulin and mucin domain containing protein-3 (TIM-3) or cytotoxic T-lymphocyte-associated antigent-4 (CTLA 4).

38. The method of any one of claims 1 to 37, wherein the first binding domain specifically binds to PD-1 and comprises a heavy chain variable domain comprising a HCDR1 having the amino acid sequence of SEQ ID NO: 56, a HCDR2 having the amino acid sequence of SEQ ID NO: 57, and a HCDR3 having the amino acid sequence of SEQ ID NO: 58, and a light chain variable domain comprising a LCDR1 having the amino acid sequence of SEQ ID NO: 59, a LCDR2 having the amino acid sequence of SEQ ID NO: 60 and a LCDR3 having the amino acid sequence of SEQ ID NO: 61.

39. The method of any one of claims 1 to 38, wherein the first binding domain specifically binds to PD-1 and comprises a heavy chain variable domain having the amino acid sequence of SEQ ID NO: 62 and a light chain variable domain having the amino acid sequence of SEQ ID NO: 64.

40. The method of any one of claims 1 to 39, wherein the first binding domain specifically binds to PD-1 and comprises a heavy chain variable domain having an amino acid sequence that is at least 90% identical to the amino acid sequence of SEQ ID NO: 7 and a light chain variable domain having an amino acid sequence that is at least 90% identical to the amino acid sequence of SEQ ID NO: 64.

41. The method of any one of claims 1 to 40, wherein the first binding domain specifically binds to PD-1 and comprises a heavy chain having the amino acid sequence of SEQ ID NO: 63 and a light chain having the amino acid sequence of SEQ ID NO: 65.

42. The method of any one of claims 1 to 41, wherein the first binding domain specifically binds to PD-1 and comprises a heavy chain having an amino acid sequence that is at least 90% identical to the amino acid sequence of SEQ ID NO: 63 and a light chain having an amino acid sequence that is at least 90% identical to the amino acid sequence of SEQ ID NO: 65.

43. The method of any one of claims 37 to 42, wherein the second binding domain specifically binds to TIGIT and comprises a heavy chain variable domain comprising a HCDR1 having the amino acid sequence of SEQ ID NO: 66, a HCDR2 having the amino acid sequence of SEQ ID NO: 67, and a HCDR3 having the amino acid sequence of SEQ ID NO: 68, and a light chain variable domain comprising a LCDR1 having the amino acid sequence of SEQ ID NO: 69, a LCDR2 having the amino acid sequence of SEQ ID NO: 70, and a LCDR3 having the amino acid sequence of SEQ ID NO: 71.

44. The method of any one of claims 37 to 43, wherein the second binding domain specifically binds to TIGIT and comprises a heavy chain variable domain having the amino acid sequence of SEQ ID NO: 72 and a light chain variable domain having the amino acid sequence of SEQ ID NO: 74.

45. The method of any one of claims 37 to 43, wherein the second binding domain specifically binds to TIGIT and comprises a heavy chain variable domain having an amino acid sequence that is at least 90% identical to the amino acid sequence of SEQ ID NO: 72 and a light chain variable domain having an amino acid sequence that is at least 90% identical to the amino acid sequence of SEQ ID NO: 74.

46. The method of any one of claims 37 to 45, wherein the second binding domain specifically binds to TIGIT and comprises a heavy chain having the amino sequence of SEQ ID NO: 73 and a light chain having the amino acid sequence of SEQ ID NO: 75.

47. The method of any one of claims 37 to 46, wherein the second binding domain specifically binds to TIGIT and comprises a heavy chain having an amino acid sequence that is at least 90% identical to the amino acid sequence of SEQ ID NO: 73 and a light chain having an amino acid sequence that is at least 90% identical to the amino acid sequence of SEQ ID NO: 75.

48. The method of any one of claims 37 to 42, wherein the second binding domain specifically binds to TIM-3.

49. The method of claim 48, wherein the second binding domain specifically binds to the C′C″ and DE loops of the immunoglobulin variable (IgV) domain of TIM-3 or binds to the PS binding cleft (FG and CC′ loops) of the IgV domain of TIM-3.

50. The method of claim 48 or claim 49, wherein the second binding domain comprises Complementarity-Determining Regions (CDRs): HCDR1, HCDR2, HCDR3, LCDR1, LCDR2, and LCDR3 comprising the amino acid sequences of SEQ ID NOs: 84, 85, 86, 87, 88, and 89, respectively, or SEQ ID NOs: 84, 85, 86, 87, 88, and 90, respectively.

51. The method of any one of claims 48 to 50, wherein the second binding domain specifically binds to at least one residue on the IgV domain of TIM-3 selected from N12, L47, R52, D53, V54, N55, Y56, W57, W62, L63, N64, G65, D66, F67, R68, K69, D71, T75, and E77 of TIM-3 (SEQ ID NO: 102).

52. The method of claim 51, wherein the second binding domain comprises a second heavy chain variable domain (VH) comprising the amino acid sequence of SEQ ID NO: 91, and a second light chain variable domain (VL) comprising the amino acid sequence of SEQ ID NO: 94.

53. The method of claim 51, wherein the second binding domain comprises a second heavy chain variable domain (VH) comprising an amino acid sequence that is at least 90% identical to the amino acid sequence of SEQ ID NO: 91, and a second light chain variable domain (VL) comprising an amino acid sequence that is at least 90% identical to the amino acid sequence of SEQ ID NO: 94.

54. The method of claim 52 or claim 53, wherein the second binding domain comprises a second heavy chain comprising the amino acid sequence of SEQ ID NO: 92, and a second light chain comprising the amino acid sequence of SEQ ID NO: 95.

55. The method of any one of claims 37 to 42, wherein the second binding domain specifically binds to CTLA-4.

56. The method claim 55, wherein the second binding domain comprises a VH CDR1 comprising the amino acid sequence of SEQ ID NO: 109, a VH CDR2 comprising the amino acid sequence of SEQ ID NO: 110, a VH CDR3 comprising the amino acid sequence of SEQ ID NO: 111, a VL CDR1 comprising the amino acid sequence of SEQ ID NO: 112, a VL CDR2 comprising the amino acid sequence of SEQ ID NO: 113, and a VL CDR3 comprising the amino acid sequence of SEQ ID NO: 114.

57. The method of claim 55 or claim 56, wherein the second binding domain comprises a heavy chain comprising the amino acid sequence set forth in SEQ ID NO: 105 and a light chain comprising the amino acid sequence set forth in SEQ ID NO: 104.

58. The method of any one of claims 1 to 57, wherein the bispecific checkpoint inhibitor is a human or humanized bispecific antibody or antigen-binding fragment thereof.

59. The method of any one of claims 1 to 58, wherein the bispecific checkpoint inhibitor comprises an IgG heavy chain constant region.

60. The method of claim 59, wherein the IgG heavy chain constant region comprises L234F, L235E and P331S.

61. The method of claim 59 or 60, wherein the IgG heavy chain constant region is an IgG1 heavy chain constant region.

62. The method of any one of claims 1 to 61, wherein the bispecific checkpoint inhibitor comprises an aglycosylated Fc region.

63. The method of any one of claims 1 to 61, wherein the bispecific checkpoint inhibitor comprises a deglycosylated Fc region.

64. The method of any one of claims 1 to 61, wherein the bispecific checkpoint inhibitor comprises an Fc region which has reduced fucosylation or is afucosylated.

65. The method of any one of claims 1 to 64, wherein the bispecific checkpoint inhibitor comprises a kappa light chain constant region.

66. The method of any one of claims 1 to 64, wherein the bispecific checkpoint inhibitor comprises a lambda light chain constant region.

67. The method of any one of claims 1 to 66, wherein the bispecific checkpoint inhibitor is an antibody.

68. The method of claim 67, wherein the antibody is an IgG antibody.

69. The method of claim 68, wherein the antibody is an IgG1 antibody.

70. The method of any one of claims 67 to 69, wherein the antibody is humanized.

71. The method of any one of claims 1 to 70, wherein the cancer comprises a cancer cell which expresses B7-H4.

72. The method of any one of claims 1 to 71, wherein the cancer is selected from ovarian cancer, breast cancer, uterine cancer, testicular cancer, bladder cancer, head and neck cancer, melanoma, renal cell carcinoma, pancreatic cancer, prostate cancer, cervical cancer, hematological cancer, endometrial cancer, cholangiocarcinoma, NSCLC (squamous and/or adenocarcinoma), gastrointestinal cancer such as gastric cancer and colorectal cancer, and lung cancer.

73. The method of any one of claims 1 to 72, wherein the cancer is a breast cancer selected from hormone receptor-positive (HR+) breast cancer, human epidermal growth factor receptor 2 positive (HER2+) breast cancer, and triple negative breast cancer (TNBC).

74. The method of any one of claims 1 to 72, wherein the cancer is homologous recombination deficient (HRD) cancer.

75. The method of claim 74, wherein the cancer comprises one or more cells having a mutation in an HRD gene selected from BRCA1, BRCA2, ATM, BRIP1, BARD1, CDK12, CHEK1, CHEK2, FANC1, PALB2, PPP2R2A, RAD51B, RAD51C, RAD51D, and RAD54L.

76. The method of claim 75, wherein the mutated HRD gene is selected from BRCA1, BRCA2, and ATM.

77. A pharmaceutical composition comprising: 1) an antibody-drug conjugate and 2) a bispecific checkpoint inhibitor, wherein the antibody-drug conjugate comprises an antibody or antigen binding fragment thereof, and a drug-linker represented by the following formula: 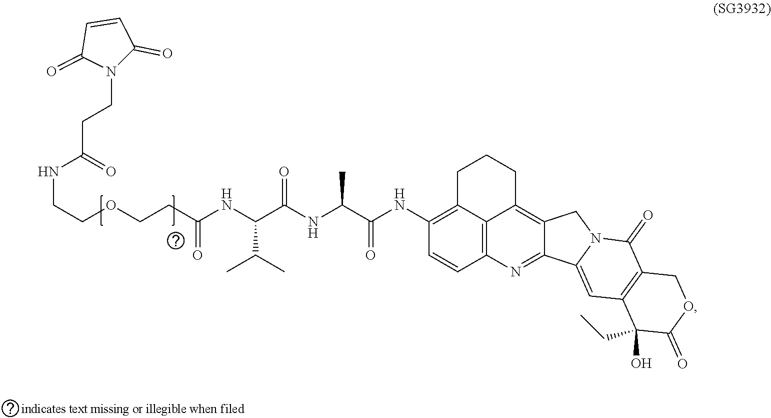
and wherein the drug-linker is conjugated to the antibody or antigen binding fragment thereof.

78. The pharmaceutical composition of claim 77, wherein the antibody or antigen binding fragment thereof is an anti-B7H4 antibody.

79. The pharmaceutical composition of claim 78, wherein the antibody or antigen binding fragment thereof binds to B7-H4 and comprises: a HCDR1, a HCDR2, a HCDR3, a LCDR1, a LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6.

80. The pharmaceutical composition of claim 79, wherein the antibody or antigen binding fragment thereof comprises a VH chain and a VL chain comprising the amino acid sequence of SEQ ID NO: 45 and SEQ ID NO: 34, respectively, or a functional variant thereof.

81. The pharmaceutical composition of claim 80, wherein the antibody or antigen binding fragment thereof comprises a heavy chain comprising the amino acid sequence of SEQ ID NO: 51; and a light chain comprising the amino acid sequence of SEQ ID NO: 44.

82. The pharmaceutical composition of claim 80, wherein the antibody or antigen binding fragment thereof comprises a heavy chain comprising the amino acid sequence of SEQ ID NO: 48; and a light chain comprising the amino acid sequence of SEQ ID NO: 44.

83. The pharmaceutical composition of any one of claims 77 to 82, wherein the bispecific checkpoint inhibitor comprises:
a) a first binding domain that specifically binds to PD-1 or PD-L1; and
b) a second binding domain that specifically binds to T cell immunoreceptor with Ig and ITIM domains (TIGIT), T-cell immunoglobulin and mucin domain containing protein-3 (TIM-3) or cytotoxic T-lymphocyte-associated antigent-4 (CTLA 4).

84. The pharmaceutical composition of claim 83, wherein the first binding domain specifically binds to PD-1 and comprises a heavy chain variable domain comprising a HCDR1 having the amino acid sequence of SEQ ID NO: 56, a HCDR2 having the amino acid sequence of SEQ ID NO: 57, and a HCDR3 having the amino acid sequence of SEQ ID NO: 58, and a light chain variable domain comprising a LCDR1 having the amino acid sequence of SEQ ID NO: 59, a LCDR2 having the amino acid sequence of SEQ ID NO: 60 and a LCDR3 having the amino acid sequence of SEQ ID NO: 61.

85. The pharmaceutical composition of claim 84, wherein the first binding domain specifically binds to PD-1 and comprises a heavy chain variable domain having the amino acid sequence of SEQ ID NO: 62 and a light chain variable domain having the amino acid sequence of SEQ ID NO: 64.

86. The pharmaceutical composition of claim 84, wherein the first binding domain specifically binds to PD-1 and comprises a heavy chain variable domain having an amino acid sequence that is at least 90% identical to the amino acid sequence of SEQ ID NO: 62 and a light chain variable domain having an amino acid sequence that is at least 90% identical to the amino acid sequence of SEQ ID NO: 64.

87. The pharmaceutical composition of claim 84, wherein the first binding domain specifically binds to PD-1 and comprises a heavy chain having the amino acid sequence of SEQ ID NO: 63 and a light chain having the amino acid sequence of SEQ ID NO: 65.

88. The pharmaceutical composition of claim 84, wherein the first binding domain specifically binds to PD-1 and comprises a heavy chain having an amino acid sequence that is at least 90% identical to the amino acid sequence of SEQ ID NO: 63 and a light chain having an amino acid sequence that is at least 90% identical to the amino acid sequence of SEQ ID NO: 65.

89. The pharmaceutical composition of any one of claims 77 to 88, wherein the second binding domain specifically binds to TIGIT and comprises a heavy chain variable domain comprising a HCDR1 having the amino acid sequence of SEQ ID NO: 66, a HCDR2 having the amino acid sequence of SEQ ID NO: 67, and a HCDR3 having the amino acid sequence of SEQ ID NO: 68, and a light chain variable domain comprising a LCDR1 having the amino acid sequence of SEQ ID NO: 69, a LCDR2 having the amino acid sequence of SEQ ID NO: 70, and a LCDR3 having the amino acid sequence of SEQ ID NO: 71.

90. The pharmaceutical composition of any one of claims 77 to 89, wherein the second binding domain specifically binds TIGIT and comprises a heavy chain variable domain having the amino acid sequence of SEQ ID NO: 72 and a light chain variable domain having the amino acid sequence of SEQ ID NO: 74.

91. The pharmaceutical composition of any one of claims 77 to 89, wherein the second binding domain specifically binds to TIGIT and comprises a heavy chain variable domain having an amino acid sequence that is at least 90% identical to the amino acid sequence of SEQ ID NO: 72 and a light chain variable domain having an amino acid sequence that is at least 90% identical to the amino acid sequence of SEQ ID NO: 74.

92. The pharmaceutical composition of any one of claims 77 to 91, wherein the second binding domain specifically binds to TIGIT and comprises a heavy chain having the amino sequence of SEQ ID NO: 73 and a light chain having the amino acid sequence of SEQ ID NO: 75.

93. The pharmaceutical composition of any one of claims 77 to 91, wherein the second binding domain specifically binds to TIGIT and comprises a heavy chain having an amino acid sequence that is at least 90% identical to the amino acid sequence of SEQ ID NO: 73 and a light chain having an amino acid sequence that is at least 90% identical to the amino acid sequence of SEQ ID NO: 75.

94. The pharmaceutical composition of any one of claims 77 to 88, wherein the second binding domain specifically binds to TIM-3.

95. The pharmaceutical composition of claim 94, wherein the second binding domain specifically binds to TIM-3, and wherein the second binding domain specifically binds to the C′C″ and DE loops of the immunoglobulin variable (IgV) domain of TIM-3 or binds to the PS binding cleft (FG and CC′ loops) of the IgV domain of TIM-3.

96. The pharmaceutical composition of claim 94 or claim 95, wherein the second binding domain comprises Complementarity-Determining Regions (CDRs): HCDR1, HCDR2, HCDR3, LCDR1, LCDR2, and LCDR3 comprising the amino acid sequences of SEQ ID NOs: 84, 85, 86, 87, 88, and 89, respectively, or SEQ ID NOs: 84, 85, 86, 87, 88, and 90, respectively.

97. The pharmaceutical composition of any one of claims 94 to 96, wherein the second binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 102).

98. The pharmaceutical composition of claim 97, wherein the second binding domain comprises a heavy chain variable domain (VH) comprising the amino acid sequence of SEQ ID NO: 91, and a light chain variable domain (VL) comprising the amino acid sequence of SEQ ID NO: 94.

99. The pharmaceutical composition of claim 98, wherein the second binding domain comprises a heavy chain comprising the amino acid sequence of SEQ ID NO: 92, and a light chain comprising the amino acid sequence of SEQ ID NO: 95.

100. The pharmaceutical composition of any one of claims 77 to 88, wherein the second binding domain specifically binds to CTLA-4.

101. The pharmaceutical composition of claim 100, wherein the second binding domain comprises a VH CDR1 comprising the amino acid sequence of SEQ ID NO: 109, a VH CDR2 comprising the amino acid sequence of SEQ ID NO: 110, a VH CDR3 comprising the amino acid sequence of SEQ ID NO: 111, a VL CDR1 comprising the amino acid sequence of SEQ ID NO: 112, a VL CDR2 comprising the amino acid sequence of SEQ ID NO: 113, and a VL CDR3 comprising the amino acid sequence of SEQ ID NO: 114.

102. The pharmaceutical composition of claim 100 or claim 101, wherein the bispecific checkpoint inhibitor comprises a heavy chain comprising the amino acid sequence set forth in SEQ ID NO: 105 and a light chain comprising the amino acid sequence set forth in SEQ ID NO: 104.

103. A kit comprising the pharmaceutical composition of any one of claims 77 to 102.

104. Use of an antibody-drug conjugate in the manufacture of a medicament for use in combination with a bispecific checkpoint inhibitor, wherein the antibody-drug conjugate and the bispecific checkpoint inhibitor are as defined in any one of claims 1 to 102 for treating cancer.

105. Use of a bispecific checkpoint inhibitor in the manufacture of a medicament for use in combination with an antibody-drug conjugate, wherein the bispecific checkpoint inhibitor and the antibody-drug conjugate are as defined in any one of claims 1 to 102 for treating cancer.

106. A pharmaceutical composition as defined in any one of claims 88 to 102, for use in treating cancer.

107. The use of any one of claims 104 to 106, wherein the cancer is selected from ovarian cancer, breast cancer, uterine cancer, testicular cancer, bladder cancer, head and neck cancer, melanoma, renal cell carcinoma, pancreatic cancer, prostate cancer, cervical cancer, hematological cancer, endometrial cancer, cholangiocarcinoma, NSCLC (squamous and/or adenocarcinoma), gastrointestinal cancer such as gastric cancer and colorectal cancer, and lung cancer.
